(19) United States
(12) Patent Application Publication
(10) Pub. No.: US 2025/0051453 A1
(43) Pub. Date: Feb. 13, 2025

(54) METHODS FOR TREATING BULLOUS PEMPHIGOID USING FCRN ANTAGONISTS

(71) Applicant: argenx BV, Zwijnaarde (Ghent) (BE)

(72) Inventors: Peter Verheesen, Zwijnaarde (Ghent) (BE); Ivaylo Stoykov, Zwijnaarde (Ghent) (BE)

(21) Appl. No.: 18/927,244

(22) Filed: Oct. 25, 2024

Related U.S. Application Data
(63) Continuation of application No. PCT/EP2023/061012, filed on Apr. 26, 2023.
(60) Provisional application No. 63/363,626, filed on Apr. 26, 2022.

(51) Int. Cl. C07K 16/28; A61K 38/47; A61K 39/00; A61K 45/06; A61P 17/00; G01N 33/564

(52) U.S. Cl. CPC C07K 16/283; A61K 38/47; A61K 45/06; A61P 17/00; C12Y 302/01035; G01N 33/564; A61K 2039/505; A61K 2039/54; A61K 2039/545; G01N 2800/20; G01N 2800/52

(57) ABSTRACT

Provided herein are methods of treating bullous pemphigoid using an effective amount of a human neonatal Fc receptor (FcRn) antagonist. FcRn antagonists for use in the treatment of bullous pemphigoid and for use in the manufacture of a medicament for the treatment of bullous pemphigoid are also provided herein.

### CROSS REFERENCE TO RELATED APPLICATIONS

This application is a Continuation of International Patent Application No. PCT/EP2023/061012, filed Apr. 26, 2023, which claims the benefit of and priority to U.S. Provisional Patent Application No. 63/363,626, filed on Apr. 26, 2022, the contents of each of which are incorporated herein by reference in their entirety.

### SUBMISSION OF SEQUENCE LISTING XML

The content of the following submission on Sequence Listing XML is incorporated herein by reference in its entirety: a computer readable form (CRF) of the Sequence Listing (file name: P236301WO00-sequence listing.xml, date created: Apr. 25, 2023, size: 37,959 bytes).

### FIELD

The present disclosure relates to methods of treating bullous pemphigoid (BP) using an antagonist of human neonatal Fc receptor (FcRn), which in certain embodiments is efgartigimod.

### BACKGROUND

Bullous pemphigoid (BP) is the most common autoimmune blistering skin disease, with an estimated annual incidence of 2.4 to 23 cases per million individuals. However, among people >80 years of age, the incidence ranges from 190 to 312 cases per million individuals. The disease has a poor prognosis, which is directly attributable to the advanced age of the patient population and the comorbidities that can be present in this population. A recent meta-analysis calculated that the relative risk of death among patients with BP during a 1-year period is 2.93 times greater that of the general worldwide population (adjusted for age and sex). Tedbirt B et al., JAMA Dermatol. 2021; 157 (4): 421-430. Studies examining the all-cause mortality among patients with BP beyond 1 year have reported higher death rates in the first year (20% to 27%) than in subsequent years (9% to 16%). The main causes of death were infections and cardiovascular disorders, which are attributable to both the older age of patients with BP and their use of corticosteroids and/or immunosuppressants.

In BP, the disruption of the dermal-epidermal junction by pathogenic autoantibodies results in large, tense bullae on the skin, progressing to epidermal erosion, crusting, and urticarial plaque formation. Without treatment, BP ultimately progresses to life-threatening complications, including (but not limited to) severe infections, functional impairment of vital organs, and iatrogenic conditions.

Corticosteroids (either topical or oral) are currently the first-line therapy for the treatment of patients with BP. For BP that is either localized or mild/moderate in severity, medical authorities recommend application of high-dose topical corticosteroids of strong potency (such as clobetasol propionate at doses from 20 to 40 g/day). Unfortunately, the use of topical corticosteroids at such high doses results in a substantial systemic absorption and severe skin atrophy.

For patients who have widespread or moderate-to-severe BP, oral corticosteroid (OCS) therapy is the standard of care among the most practitioners. Unfortunately, OCS cause significant toxicity in patients with BP, because the cumulative corticosteroid exposure that is typically required to successfully manage the disease is high. Thus, physicians who treat patients with BP must provide the optimal corticosteroid dose to successfully manage the disease, achieve control of disease activity (CDA) or complete/partial remission (CR/PR), and prevent subsequent relapse, while also mitigating the adverse effects of corticosteroids by minimizing the amount that they prescribe to their patients.

Alternative adjuvant treatments to corticosteroids have been evaluated in patients with severe BP or in those who expressed corticosteroid resistance, including IVIg, plasma exchange, rituximab, omalizumab, dapsone, and tetracyclines. These treatments have either shown inconsistent results, proven to be prohibitively complicated and/or expensive, induced significant side effects in their respective populations of patients with BP, or produced encouraging results only to have significant portions of those who responded to treatment ultimately relapse shortly thereafter.

Accordingly, there is an unmet need in the art for new and improved BP treatment options that provide rapid CDA and remission, minimize (or even prevent) relapse, and permit tapering or elimination of corticosteroid treatment.

### SUMMARY

The instant disclosure is broadly directed to methods for treating bullous pemphigoid (BP) with neonatal Fc receptor (FcRn) antagonists.

In an aspect, provided herein is a method for treating BP in a subject in need thereof, the method comprising administering to the subject an effective amount of a human FcRn antagonist.

In some embodiments, the FcRn antagonist comprises two, three, or four FcRn binding regions.

In some embodiments, the FcRn antagonist comprises or consists of a variant Fc region or FcRn binding fragment thereof. In some embodiments, the variant Fc region or FcRn binding fragment thereof binds to FcRn with a higher affinity at pH 6.0 as compared to a corresponding wild-type Fc region. In some embodiments, the variant Fc region or FcRn binding fragment thereof binds to FcRn with a higher affinity at pH 7.4 as compared to a corresponding wild-type Fc region.

In some embodiments, the variant Fc region comprises or consists of two Fc domains which form a homodimer or heterodimer. In some embodiments, the FcRn antagonist comprises an Fc region comprising amino acids Y, T, E, K, F, and Y at EU positions 252, 254, 256, 433, 434, and 436, respectively.

In some embodiments, the variant Fc region comprises or consists of a first Fc domain and a second Fc domain which form a homodimer or heterodimer. In some embodiments, the first Fc domain and/or the second Fc domain comprise amino acids Y, T, E, K, and F at EU positions 252, 254, 256, 433, and 434, respectively. In some embodiments, the first Fc domain and/or the second Fc domain comprise amino acids Y, T, E, K, F, and Y at EU positions 252, 254, 256, 433, 434, and 436, respectively.

In some embodiments, the amino acid sequence of at least one of the Fc domains is selected from the group consisting of SEQ ID NO: 1, SEQ ID NO: 2, and SEQ ID NO: 3. In some embodiments, the amino acid sequence of both of the Fc domains is independently selected from the group consisting of SEQ ID NO: 1, SEQ ID NO: 2, and SEQ ID NO: 3. In some embodiments, the FcRn antagonist is efgartigimod.

In some embodiments, the first Fc domain and/or the second Fc domain comprise an amino acid sequence independently selected from the group consisting of SEQ ID NO: 1, SEQ ID NO: 2, and SEQ ID NO: 3. In some embodiments, the first Fc domain and the second Fc domain comprise an amino acid sequence independently selected from the group consisting of SEQ ID NO: 1, SEQ ID NO: 2, and SEQ ID NO: 3. In some embodiments, the FcRn antagonist is efgartigimod.

In some embodiments, the FcRn antagonist is an anti-FcRn antibody. In some embodiments, the anti-FcRn antibody is selected from the group consisting of rozanolixizumab, nipocalimab, orilanolimab, and batoclimab.

In some embodiments, the FcRn antagonist is administered to the subject at a fixed dose of 20 mg to 20,000 mg or at a dose of 0.2 mg/kg to 200 mg/kg. In some embodiments, the FcRn antagonist is administered to the subject at a fixed dose of 200 mg to 20,000 mg or at a dose of 2 mg/kg to 200 mg/kg.

In some embodiments, the FcRn antagonist is administered subcutaneously once weekly, once every two weeks, once every three weeks, once every four weeks, once monthly, or once every six weeks. In some embodiments, the FcRn antagonist is administered subcutaneously at a fixed dose of 20 mg to 20,000 mg once weekly, once every two weeks, once every three weeks, once every four weeks, once monthly, or once every six weeks. In some embodiments, the FcRn antagonist is administered subcutaneously at a fixed dose of 200 mg to 20,000 mg once weekly, once every two weeks, once every three weeks, once every four weeks, once monthly, or once every six weeks. In some embodiments, the FcRn antagonist is administered subcutaneously at a fixed dose of 750 mg to 3000 mg once weekly, once every two weeks, once every three weeks, once every four weeks, once monthly, or once every six weeks. In some embodiments, the FcRn antagonist is administered subcutaneously at a fixed dose of 1000 mg or 2000 mg once weekly, once every two weeks, once every three weeks, once every four weeks, once monthly, or once every six weeks.

In some embodiments, the FcRn antagonist is first administered subcutaneously at a fixed dose of about 1000 mg twice on the same day. In some embodiments, the FcRn antagonist is administered subcutaneously at a fixed dose of about 2000 mg for the first two administrations and is subsequently administered subcutaneously at a fixed dose of about 1000 mg. In some embodiments, the FcRn antagonist is administered subcutaneously at a fixed dose of 2000 mg for the first two administrations and is subsequently administered subcutaneously at a fixed dose of 1000 mg.

In some embodiments, the first two administrations of the FcRn antagonist are subcutaneous administrations at a fixed dose of about 1000 mg administered twice on the same day. In some embodiments, the first two administrations of the FcRn antagonist are subcutaneous administrations at a fixed dose of 1000 mg administered twice on the same day.

In some embodiments, the FcRn antagonist is efgartigimod. In some embodiments, efgartigimod is administered subcutaneously at a fixed dose of 800 to 1200 mg once weekly. In some embodiments, efgartigimod is administered subcutaneously at a fixed dose of about 1000 mg once weekly. In some embodiments, efgartigimod is administered subcutaneously at a fixed dose of 1000 mg once weekly.

In some embodiments, the FcRn antagonist is co-formulated with hyaluronidase and administered subcutaneously. In some embodiments, the hyaluronidase is recombinant human hyaluronidase PH20 (rHuPH20). In some embodiments, the rHuPH20 is administered at a dose of 2000 U/mL once weekly. In some embodiments, the rHuPH20 is administered at about 11,000 U or about 22,000 U once weekly.

In some embodiments, the FcRn antagonist is administered intravenously once weekly or once every two weeks. In some embodiments, the FcRn antagonist is administered intravenously at a dose of from 0.2 mg/kg to 200 mg/kg once weekly or once every two weeks. In some embodiments, the FcRn antagonist is administered intravenously at a dose of from 2 mg/kg to 200 mg/kg once weekly or once every two weeks. In some embodiments, the FcRn antagonist is administered intravenously at a dose of from 3 mg/kg to 60 mg/kg once weekly or once every two weeks. In some embodiment, the FcRn antagonist is administered intravenously at a dose of 5 mg/kg once weekly or once every two weeks. In some embodiments, the FcRn antagonist is administered intravenously at a dose of 10 mg/kg once weekly or once every two weeks. In some embodiments, the FcRn antagonist is administered intravenously at a dose of 25 mg/kg once weekly or once every two weeks.

In some embodiments, the FcRn antagonist is first administered intravenously and is subsequently administered subcutaneously. In some embodiments, the FcRn antagonist is first administered intravenously and is subsequently administered subcutaneously at fixed dose of 20 mg to 20,000 mg once weekly, once every two weeks, once every three weeks, once every four weeks, once monthly, or once every six weeks. In some embodiments, the FcRn antagonist is first administered intravenously and is subsequently administered subcutaneously at fixed dose of 200 mg to 20,000 mg once weekly, once every two weeks, once every three weeks, once every four weeks, once monthly, or once every six weeks. In some embodiments, the FcRn antagonist is first administered intravenously and is subsequently administered subcutaneously at fixed dose of 1000 mg or 2000 mg once weekly, once every two weeks, once every three weeks, once every four weeks, once monthly, or once every six weeks.

In some embodiments, the FcRn antagonist is administered for 52 weeks or less. In some embodiments, the FcRn antagonist is administered for 36 weeks or less.

In some embodiments, the FcRn antagonist is administered for at least 26 weeks. In some embodiments, the FcRn antagonist is administered for at least 36 weeks.

In some embodiments, the methods disclosed herein further comprise administering to the subject a dose of corticosteroid. In some embodiments, the dose of corticosteroid is reduced after disease control or complete remission has been achieved. In some embodiments, the dose of corticosteroid is reduced after disease control has been sustained for at least two weeks.

In some embodiments, following two weeks of sustained disease control, the dose of corticosteroid is decreased from a prednisone equivalent dose of 0.5 mg/kg/day to a prednisone equivalent dose of 0.3 mg/kg/day, from a prednisone equivalent dose of 0.3 mg/kg/day to a prednisone equivalent dose of 0.2 mg/kg/day, from a prednisone equivalent dose of 0.2 mg/kg/day to a prednisone equivalent dose of 0.15 mg/kg/day, and from a prednisone equivalent dose of 0.15 mg/kg/day to a prednisone equivalent dose of 0.1 mg/kg/day.

In some embodiments, following two weeks of sustained disease control, the dose of corticosteroid is decreased from a prednisone equivalent dose of 0.75 mg/kg/day to a prednisone equivalent dose of 0.5 mg/kg/day, from a prednisone equivalent dose of 0.5 mg/kg/day to a prednisone equivalent dose of 0.3 mg/kg/day, from a prednisone equivalent dose of 0.3 mg/kg/day to a prednisone equivalent dose of 0.2 mg/kg/day, from a prednisone equivalent dose of 0.2 mg/kg/day to a prednisone equivalent dose of 0.15 mg/kg/day and from a prednisone equivalent dose of 0.15 mg/kg/day to a prednisone equivalent dose of 0.1 mg/kg/day.

In some embodiments, following two weeks of sustained disease control, the dose of corticosteroid is decreased from a prednisone equivalent dose of 1 mg/kg/day to a prednisone equivalent dose of 0.75 mg/kg/day, from a prednisone equivalent dose of 0.75 mg/kg/day to a prednisone equivalent dose of 0.5 mg/kg/day, from a prednisone equivalent dose of 0.5 mg/kg/day to a prednisone equivalent dose of 0.3 mg/kg/day, from a prednisone equivalent dose of 0.3 mg/kg/day to a prednisone equivalent dose of 0.2 mg/kg/day, from a prednisone equivalent dose of 0.2 mg/kg/day to a prednisone equivalent dose of 0.15 mg/kg/day, and from a prednisone equivalent dose of 0.15 mg/kg/day to a prednisone equivalent dose of 0.1 mg/kg/day.

In some embodiments, each dose of corticosteroid is maintained for at least two weeks before the dose is further reduced. In some embodiments, each dose of corticosteroid is maintained for at least two weeks provided that no new lesions appear.

In some embodiments, once the subject is on a prednisone equivalent dose of 0.1 mg/kg/day, the daily dose of corticosteroid is further reduced by a prednisone equivalent dose of 2.5 mg every two weeks for one or more two-week periods. In some embodiments, the daily dose of corticosteroid is decreased by a prednisone equivalent dose of 2.5 mg every two weeks for one or more two-week periods until the daily dose of corticosteroid is zero, thereby resulting in discontinuation of corticosteroid therapy. In some embodiments, each daily dose is maintained for two weeks provided that no new lesions appear.

In some embodiments, once the subject is on a prednisone equivalent dose of 0.1 mg/kg/day, the daily dose of corticosteroid is further reduced by a prednisone equivalent dose of 1 mg every week for one or more one-week periods. In some embodiments, the daily dose of corticosteroid is decreased by a prednisone equivalent dose of 1 mg every week for one or more one-week periods until the daily dose of corticosteroids is zero, thereby resulting in discontinuation of corticosteroid therapy. In some embodiments, each daily dose is maintained for one week provided that no new lesions appear.

In some embodiments, the corticosteroid is prednisone.

In some embodiments, the BP is selected from the group consisting of newly diagnosed BP, relapsing BP, and refractory BP.

In some embodiments, the subject is diagnosed with moderate-to-severe BP, mild BP, moderate BP, or severe BP.

In some embodiments, the subject has a detectable serum level of an anti-BP180 antibody or an anti-BP230 antibody or both. In some embodiments, the subject shows a reduction in serum level of the anti-BP180 antibody or anti-BP230 antibody following administration of the FcRn antagonist. In some embodiments, the reduction in the serum level of the anti-BP180 antibody or anti-BP230 antibody following administration of the FcRn antagonist is at least 10%, at least 25%, at least 50%, at least 75%, at least 80%, at least 90%, at least 95%, at least 96%, at least 97%, at least 98%, or at least 99% of the subject's serum level of the anti-BP180 antibody or anti-BP230 antibody prior to administration of the FcRn antagonist. In some embodiments, the serum level of the anti-BP180 antibody or anti-BP230 antibody is undetectable after administration of the FcRn antagonist.

In some embodiments, the FcRn antagonist is administered until disease control, partial remission, or complete remission is achieved. In some embodiments, the subject achieves complete remission. In some embodiments, the subject achieves complete remission within 1 month, within 2 months, within 3 months, within 4 months, within 5 months, within 6 months, within 9 months, or within 1 year. In some embodiments, the subject achieves complete remission within about 3 to 6 months. In some embodiments, complete remission is sustained in the subject for at least 2 months. In some embodiments, complete remission is sustained in the subject for least 6 months.

In some embodiments, the subject remains in complete remission in the absence of oral corticosteroids. In some embodiments, the subject remains in complete remission in the absence of oral corticosteroids for at least eight weeks. In some embodiments, the subject remains in complete remission in the absence of corticosteroids for at least eight weeks at the end of a 36-week treatment period.

In some embodiments, BP relapse is prevented after achieving remission from BP, wherein BP relapse is prevented for at least 36 weeks after initiation of the FcRn antagonist administration.

In some embodiments, the subject has an IGA-BP score of 0 or 1 after administration of the FcRn antagonist. In some embodiments, the subject has an IGA-BP score of 0 or 1 in the absence of oral corticosteroids for at least eight weeks. In some embodiments, the subject has an IGA-BP score of 0 or 1 in the absence of oral corticosteroids for at least eight weeks at the end of a 36-week treatment period.

In some embodiments, the subject has an IGA-BP score of 0 after administration of the FcRn antagonist. In some embodiments, the subject has an IGA-BP score of 0 in the absence of oral corticosteroids for at least eight weeks. In some embodiments, the subject has an IGA-BP score of 0 in the absence of oral corticosteroids for at least eight weeks at the end of a 36-week treatment period.

Also provided herein is an FcRn antagonist for use in the treatment of BP, wherein the treatment is performed according to any of the methods disclosed herein.

Also provided herein is an FcRn antagonist for use in the manufacture of a medicament for the treatment of BP, wherein the treatment is performed according to any of the methods disclosed herein.

Also provided herein is a method for monitoring efficacy of treatment of BP in a subject following treatment with a first FcRn antagonist, the method comprising: a) measuring in vitro a serum level of an anti-BP180 antibody and/or anti-BP230 antibody in a blood sample taken from the subject; and b) comparing the serum level of the anti-BP180 antibody and/or anti-BP230 antibody to a reference value associated with BP in the subject, wherein the treatment is not effective if the serum level of the anti-BP180 antibody and/or anti-BP230 antibody in the sample is greater than or equal to the reference value, and wherein the treatment is effective if the serum level of the anti-BP180 antibody and/or anti-BP230 antibody is less than the reference value.

Also provided herein is a method of treating BP in a subject that has received a first FcRn antagonist and is receiving a corticosteroid dosing regimen, the method comprising: a) administering to the subject an effective amount of a second FcRn antagonist; b) measuring in vitro a serum level of an anti-BP180 antibody and/or anti-BP230 antibody in a blood sample taken from the subject; and c) comparing the serum level of the anti-BP180 antibody and/or anti-BP230 antibody to a reference value associated with BP in the subject, wherein the corticosteroid dosing regimen is maintained if the serum level of the anti-BP180 antibody and/or anti-BP230 antibody in the sample is greater than or equal to the reference value, or wherein the corticosteroid dosing regimen is tapered if the serum level of the anti-BP180 antibody and/or anti-BP230 antibody is less than the reference value.

Also provided herein is a method for treating BP in a subject comprising: (a) administering to the subject one or more initial doses of an effective amount of a first FcRn antagonist; and (b) administering to the subject one or more further doses of an effective amount of a second FcRn antagonist if the serum level of an anti-BP180 antibody and/or anti-BP230 antibody in the subject after step (a) is greater than or equal to a reference value associated with BP in the subject, or discontinuing treatment with the first FcRn antagonist if the serum level of the anti-BP180 antibody and/or anti-BP230 antibody in the subject after step (a) is less than a reference value associated with active disease in the subject.

Also provided herein is a method for treating BP in a subject, the method comprising: administering to the subject an effective amount of a second FcRn antagonist, wherein the BP has relapsed in the subject following prior therapy with a first FcRn antagonist, and wherein the subject has a serum level of an anti-BP180 antibody and/or anti-BP230 antibody that is greater than or equal to a reference value associated with BP in the subject.

Also provided herein is a method for determining if a subject that has previously been treated for BP using a first FcRn antagonist requires further treatment with a second FcRn antagonist, the method comprising: a) measuring in vitro the serum level of an anti-BP180 antibody and/or anti-BP230 antibody in a blood sample taken from the subject; and b) comparing the serum level of the anti-BP180 antibody and/or anti-BP230 antibody to a reference value associated with BP in the subject, wherein if the serum level of the anti-BP180 antibody and/or anti-BP230 antibody in the sample is greater than or equal to the reference value, then the subject is in need of further treatment with the second FcRn antagonist.

In some embodiments, the subject was previously treated with the first FcRn antagonist at a fixed dose of about 200 mg to about 20,000 mg or at a dose of 2 mg/kg to 200 mg/kg.

In some embodiments, the subject was previously treated with the first FcRn antagonist at a dose of from 20 mg/kg to 100 mg/kg, administered intravenously.

In some embodiments, the subject was previously treated with the first FcRn antagonist at a dose of from 3 mg/kg to 60 mg/kg, administered intravenously.

In some embodiments, the effective amount of the second FcRn antagonist is a higher dose than the previous treatment with the first FcRn antagonist.

In some embodiments, the effective amount of the second FcRn antagonist is a lower dose than the previous treatment with the first FcRn antagonist.

In some embodiments, the effective amount of the second FcRn antagonist is administered at a fixed dose of about 200 mg to about 20,000 mg or at a dose of 2 mg/kg to 200 mg/kg.

In some embodiments, the effective amount of the second FcRn antagonist is administered subcutaneously at a fixed dose of about 750 mg to about 3000 mg once weekly, once every two weeks, once every three weeks, once every four weeks, once monthly, or once every six weeks.

In some embodiments, the effective amount of the second FcRn antagonist is administered subcutaneously at a fixed dose of about 1000 mg or about 2000 mg once weekly, once every two weeks, once every three weeks, once every four weeks, once monthly or once every six weeks.

In some embodiments, the first FcRn antagonist and the second FcRn antagonist are each the same FcRn antagonist. In some embodiments, the FcRn antagonist comprises or consists of a variant Fc region or FcRn binding fragment thereof. In some embodiments, the variant Fc region or FcRn binding fragment thereof binds to FcRn with a higher affinity at pH 6.0 and/or 7.4 as compared to a corresponding wild-type Fc region. In some embodiments, the variant Fc region comprises or consists of two Fc domains which form a homodimer or heterodimer. In some embodiments, the FcRn antagonist is an Fc region comprising amino acids Y, T, E, K, F, and Y at EU positions 252, 254, 256, 433, 434, and 436, respectively. In some embodiments, the FcRn antagonist is efgartigimod.

In some embodiments, the first FcRn antagonist and the second FcRn antagonist are each a different FcRn antagonist. In some embodiments, the first FcRn antagonist or the second FcRn antagonist comprises or consists of a variant Fc region or FcRn binding fragment thereof. In some embodiments, the variant Fc region or FcRn binding fragment thereof binds to FcRn with a higher affinity at pH 6.0 and/or 7.4 as compared to a corresponding wild-type Fc region. In some embodiments, the variant Fc region comprises or consists of two Fc domains which form a homodimer or heterodimer. In some embodiments, the first FcRn antagonist or the second FcRn antagonist is an Fc region comprising amino acids Y, T, E, K, F, and Y at EU positions 252, 254, 256, 433, 434, and 436, respectively. In some embodiments, the first FcRn antagonist or the second FcRn antagonist is efgartigimod.

In some embodiments, the amino acid sequence of at least one of the Fc domains is selected from the group consisting of SEQ ID NO: 1, SEQ ID NO: 2, and SEQ ID NO: 3. In some embodiments, the amino acid sequence of both of the Fc domains is independently selected from the group consisting of SEQ ID NO: 1, SEQ ID NO: 2, and SEQ ID NO: 3.

In some embodiments, the first FcRn antagonist is an anti-FcRn antibody and the second FcRn antagonist is efgartigimod.

In some embodiments, the first FcRn antagonist is an anti-FcRn antibody and the second FcRn antagonist comprises the amino acid sequence of SEQ ID NO: 1, 2, or 3.

In some embodiments, the anti-FcRn antibody is rozanolixizumab, nipocalimab, orilanolimab, or batoclimab.

In some embodiments, the patient has not been previously treated with efgartigimod.

In some embodiments, the subject has one or more physical symptoms of BP following treatment with the first FcRn antagonist.

In some embodiments, the subject has a serum level of an anti-BP180 antibody or anti-BP230 antibody that is associated with relapse of BP.

Also provided herein is a method of treating BP in a subject in need thereof, the method comprising administering to the subject an effective amount of a human FcRn antagonist and a corticosteroid, wherein a cumulative oral dose of the corticosteroid administered to the subject does not exceed 100 mg/kg, does not exceed 75 mg/kg, does not exceed 50 mg/kg, or does not exceed 25 mg/kg.

Also provided herein is a method of treating BP in a subject in need thereof, the method comprising administering to the subject an effective amount of a human FcRn antagonist and a corticosteroid, wherein an oral prednisone equivalent dose of the corticosteroid administered to the subject does not exceed 1 mg/kg/day, does not exceed 0.75 mg/kg/day, does not exceed 0.5 mg/kg/day, does not exceed 0.3 mg/kg/day, does not exceed 0.25 mg/kg/day, does not exceed 0.2 mg/kg/day, does not exceed 0.15 mg/kg/day, or does not exceed 0.1 mg/kg/day.

Also provided herein is a method of treating BP in a subject in need thereof, the method comprising administering to the subject an effective amount of a human FcRn antagonist and a corticosteroid, wherein an oral prednisone equivalent dose of the corticosteroid administered to the subject does not exceed 12.5 mg/day or does not exceed 10 mg/day.

### BRIEF DESCRIPTION OF THE DRAWINGS

FIG. 1A-1B shows data of clinical disease activity in an anti-hNC16A IgG-induced acute preventative BP mouse model. Adult hNC16A mice were injected intraperitoneally (i.p.) with normal human control IgG (NHC) or anti-hNC16A IgG (250 μg/g body weight), followed by intravenous (i.v.) injection with an efgartigimod analogue, HEL-ABDEG (60 μg/g body weight), or PBS control, and examined clinically 3 days post injection. FIG. 1A: injection of anti-hNC16A IgG antibodies (α-NC16A IgG) results in development of ear skin lesions, whereas HEL-ABDEG treated mice skin looks similar to mice injected with normal human control (NHC) IgG. FIG. 1B shows the disease activity scoring. *p<0.01, n=8 for each group.

FIG. 2 shows histopathology data from skin lesions in an anti-hNC16A IgG-induced acute preventative BP mouse model. Representative images of routine histology examination of mouse skin with H/E staining (top panels marked a-c: 200× magnification; bottom panels marked d-f: 400× magnification). Mice injected with pathogenic anti-hNC16A IgG that were treated with HEL-ABDEG showed no dermal-epidermal separation (top middle panel), and significantly reduced skin neutrophil infiltration (bottom middle panel) compared to mice that received PBS control (left panels). Mice injected with normal human IgG control (NHC) followed by PBS treatment show no dermal-epidermal separation.

FIG. 3 shows indirect immunofluorescent (IIF) staining of lesions in the acute preventative BP mouse model shows reduced neutrophil infiltration in HEL-ABDEG treated mice. The skin of mice treated with HEL-ABDEG exhibited significantly reduced infiltrating neutrophils (2^{nd} column from the left, middle panel marked f) compared to PBS-treated control mice (2^{nd} column from the left, top panel marked b) as evidenced by IIF (200× magnification). NHC IgG injected skin shows no neutrophil infiltration (2^{nd} column from the left, bottom panel marked j).

FIG. 4A-4B shows data from an anti-hNC16A IgG-induced acute therapeutic BP mouse model. Adult hNC16A mice were injected i.p. with anti-hNC16A (250 μg/g body weight). Three days later, mice were injected i.v. with HEL-ABDEG (60 μg/g body weight) or PBS control. The mice were examined on day 10 and 17 (7 and 14 days post-HEL-ABDEG injection, respectively). On day 10 (7 days post-treatment), mice treated with HEL-ABDEG exhibited significant reduction in clinical disease activity (FIG. 4A) and less infiltrating neutrophils as evidenced by IIF (FIG. 4B) compared to the control group. For FIG. 4A, *p=0.026, n=8 for PBS control group, n=6 for HEL-ABDEG treatment group.

FIG. 5 is a schematic of a 2-part, phase 2/3 study protocol to investigate the efficacy, safety, tolerability, immunogenicity, participant-reported outcome measures (including QOL assessments), PK, and PD of efgartigimod PH20 SC in adult participants with moderate-to-severe BP (BALLAD). BP: bullous pemphigoid; CDA: control of disease activity; CR: complete remission; EFG: efgartigimod; HCP: health care provider; OCS: oral corticosteroids; PR: partial remission; SC: subcutaneous. *Rescue therapy may also be initiated if the participant relapses twice or presents with steroid toxicity, or any severe adverse event deemed by the investigator to be related to OCS therapy. These participants will stop efgartigimod therapy but will continue attending study visits through the 36-week treatment period while receiving rescue therapy.

FIG. 6 is a schematic depicting treatment regimens for the participants in ARGX-113-2010 (the study described in Example 3). 8w=8 weeks; BL=baseline; BP=bullous pemphigoid; CR=complete remission; EFG PH20 SC=efgartigimod coformulated with rHuPH20 for subcutaneous injection (dosing formulation); EoTP-end of treatment period (visit; ARGX-113-2009); IMP=investigational medicinal product; OCS-oral corticosteroids; PR=partial remission; rHuPH20=recombinant human hyaluronidase PH20; TCS=topical corticosteroids; W36-week 36 of ARGX-113-2009 (the EoTP visit).

### DETAILED DESCRIPTION

The present disclosure provides engineered FcRn antagonists and methods for their use in treating bullous pemphigoid (BP). Advantageously, the methods disclosed herein permit rapid treatment of disease, as well as the potential to taper and even discontinue corticosteroids after achieving clinical remission.

### Definitions

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “FcRn antagonist” refers to any agent that binds specifically to FcRn and inhibits the binding of immunoglobulin to FcRn (e.g., human FcRn). In an embodiment, the FcRn antagonist is an Fc region (e.g., a variant Fc region disclosed herein) that specifically binds to FcRn through the Fc region and inhibits the binding of immunoglobulin to FcRn. In an embodiment, the FcRn antagonist is not a full-length IgG antibody. In an embodiment, the FcRn antagonist comprises an antigen binding site that binds a target antigen and a variant Fc region. In an embodiment, the FcRn antagonist is an Fc fragment comprising or consisting of an Fc region and lacking an antigen binding site. In an embodiment the term “FcRn antagonist” refers to an antibody or antigen-binding fragment thereof that specifically binds to FcRn via its antigen binding domain or via its Fc region and inhibits the binding of the Fc region of immunoglobulin (e.g., IgG autoantibodies) to FcRn.

As used herein, the terms “antibody” and “antibodies” include full-length antibodies, antigen-binding fragments of full-length antibodies, and molecules comprising antibody CDRs, VH regions, or VL regions. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single-domain antibodies (sdAb), monovalent antibodies, single chain antibodies or single-chain Fvs (scFv), camelid antibodies, affibody molecules, humanized antibodies, VHH fragments, Fab fragments, F (ab′) 2 fragments, disulfide-linked Fvs (sdFv), anti-idiotypic (anti-Id) antibodies (including, e.g., anti-anti-Id antibodies), and antigen-binding fragments of any of the above. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂ₐ or IgG_{2b}) of immunoglobulin molecule.

As used herein, the term “Fc domain” refers to the portion of a single immunoglobulin heavy chain beginning in the hinge region and ending at the C-terminus of the antibody. Accordingly, a complete Fc domain comprises at least a portion of a hinge (e.g., upper, middle, and/or lower hinge region) domain, a CH2 domain, and a CH3 domain. In some embodiments, the term “Fc domain” refers to the portion of a single immunoglobulin heavy chain comprising both the CH2 and CH3 domains of the antibody. In some embodiments, the Fc domain comprises at least a portion of a hinge (e.g., upper, middle, and/or lower hinge region) region, a CH2 domain, and a CH3 domain. In some embodiments, the Fc domain does not include the hinge region.

As used herein, the term “hinge region” refers to the portion of a heavy chain molecule that joins the CH1 domain to the CH2 domain. In some embodiments, the hinge region is at most 70 amino acid residues in length. In some embodiments, this hinge region comprises approximately 11-17 amino acid residues and is flexible, thus allowing the two N-terminal antigen binding regions to move independently. In some embodiments, the hinge region is 12 amino acid residues in length. In some embodiments, the hinge region is 15 amino acid residues in length. In some embodiments, the hinge region is 62 amino acid residues in length. Hinge regions can be subdivided into three distinct domains: upper, middle, and lower hinge domains. The FcRn antagonists of the instant disclosure can include all or any portion of a hinge region. In some embodiments, the hinge region is from an IgG1 antibody. In some embodiments, the hinge region comprises the amino acid sequence of EPKSCDKTHTCPPCP (SEQ ID NO: 12).

As used herein, the term “Fc region” refers to the portion of an immunoglobulin formed by the Fc domains of its two heavy chains. The Fc region can be a wild-type Fc region (native Fc region) or a variant Fc region. A native Fc region is homodimeric. The Fc region can be derived from any native immunoglobulin. In some embodiments, the Fc region is formed from an IgA, IgD, IgE, or IgG heavy chain constant region. In some embodiments, the Fc region is formed from an IgG heavy chain constant region. In some embodiments, the IgG heavy chain constant region is an IgG1, IgG2, IgG3, or IgG4 heavy chain constant region. In some embodiments, the Fc region is formed from an IgG1 heavy chain constant region. In some embodiments, the IgG1 heavy chain constant region comprises a G1m1(a), G1m2(x), G1m3(f), or G1m17 (z) allotype. See, e.g., Jefferis and Lefranc, mAbs; 2009; 1 (4): 332-338, and de Taeye et al., Front Immunol. 2020; 11:740, incorporated herein by reference in their entirety.

As used herein, the term “variant Fc region” refers to an Fc region with one or more alteration(s) relative to a native Fc region. Alterations can include amino acid substitutions, additions and/or deletions, linkage of additional moieties, and/or alteration of the native glycans. The term encompasses heterodimeric Fc regions where each of the constituent Fc domains is different. The term also encompasses single chain Fc regions where the constituent Fc domains are linked together by a linker moiety.

As used herein the term “FcRn binding fragment” refers to a portion of an Fc region that is sufficient to confer FcRn binding.

As used herein, the term “EU position” refers to the amino acid position in the EU numbering convention for the Fc region described in Edelman, G M et al., Proc. Natl. Acad. USA, 1969; 63, 78-85, and Rabat et al., in “Sequences of Proteins of Immunological Interest,” U.S. Dept. Health and Human Services, 5th edition, 1991.

As used herein, the term “baseline” refers to a measurement (e.g., a frequency of B cells, IgG levels) in a patient, e.g., in a patient's blood, prior to the first administration (e.g., intravenous, or subcutaneous administration) of a treatment (e.g., an FcRn antagonist).

As used herein, the term “autoantibody-mediated disease” refers to any disease or disorder in which the underlying pathology is caused, at least in part, by pathogenic IgG autoantibodies.

As used herein, the term “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “dose” or “dosing” refers to an amount of an agent administered to a subject in a single administration.

As used herein, the terms “fixed dose” or “flat dose” both refer to a dose that does not vary based upon a characteristic (e.g., body mass, e.g., within a set range; sex; age, e.g., within a set range; etc.) of the subject.

As used herein, the term “minimal OCS therapy” refers to an oral prednisone dosage (or prednisone equivalent dose) of ≤0.10 mg/kg/day.

As used herein, the term “prednisone equivalent dose” means a dose of prednisone or an equivalent dose of a systemic corticosteroid other than prednisone. Systemic corticosteroids are well-known and include compounds of various potencies and formulations. These are generally formulated as injectables or pills. Examples of commercially available systemic corticosteroids include, without limitation, betamethasone, cortisone, dexamethasone, hydrocortisone, methylprednisolone, prednisone, prednisolone, and triamcinolone.

As used herein, the term “control of disease activity” or “CDA” refers to the point at which new lesions cease to form and established lesions begin to heal, and pruritic symptoms start to abate.

As used herein, the term “treatment failure” refers to the absence of CDA after administration of one or more treatments for a specified length of time. In some aspects, “treatment failure” refers to the absence of CDA despite receiving at least 4 consecutive once-weekly doses of efgartigimod PH20 SC with or without concurrent BP therapy. In some aspects, “treatment failure” refers to the absence of CDA after being administered at least 4 consecutive once-weekly doses of efgartigimod with or without oral prednisone at 0.75 mg/kg/day (or equivalent OCS) for 3 weeks. Optionally, a fourth week of therapy with oral prednisone 0.75 mg/kg/day or 1 mg/kg/day (based on clinical judgment) may be added before declaring treatment failure.

As used herein, the term “transient lesions” refers to new lesions that heal within 1 week or pruritus lasting less than 1 week.

As used herein, the term “non-transient lesions” can be defined as new lesions that do not heal within 1 week or pruritus continuing longer than 1 week.

As used herein, the term “remission” refers to complete remission or partial remission.

As used herein, the term “complete remission” or “CR” refers to the absence of new lesions, complete healing of existing lesions, and absence of pruritus.

As used herein, the term “partial remission” or “PR” refers to the presence of only new transient lesions.

As used herein, the term “relapse” or “flare” refers to a patient with BP who has an appearance of physical symptoms and/or an increase of a marker of BP after a period of remission of BP. In some aspects, the term “relapse” or “flare” refers to the occurrence of any of the following in a patient who has achieved CDA: the appearance of 3 or more new lesions (such as blisters, eczematous lesions, or urticarial plaques) in 1 month (4 weeks); the appearance of at least 1 large (>10 cm diameter) eczematous lesion that does not heal within 1 week; the appearance of urticarial plaques that do not heal within 1 week; the extension/expansion of established lesions; and/or new or increased intensity of daily pruritic symptoms.

As used herein, the term “subject” or “patient” or “participant” includes any human or non-human animal. In an embodiment, the subject or patient or participant is a human or non-human mammal. In an embodiment, the subject or patient or participant is a human.

As used herein, the term “about” or “approximately” when referring to a measurable value, such as a dosage, encompasses variations of +20%, +15%, +10%, +5%, +1%, or +0.1% of a given value or range, as are appropriate to perform the methods disclosed herein.

### Bullous Pemphigoid (BP)

BP is a subepidermal autoimmune blistering disease (AIBD) that predominantly affects the elderly. It is a chronic disease that significantly impacts morbidity and quality of life (QOL); additionally, the disease can worsen spontaneously, even when the patient is treated with the current standard of care. The pathogenesis of BP is driven by IgG and IgE autoantibodies against the hemidesmosomal proteins BP180 and BP230, acting as key antigens for pathogenic autoantibodies. These pathogenic autoantibodies are understood to result in direct interference of autoantigen adherence, activation of complement, recruitment of inflammatory cells (e.g., eosinophils), and release of proteolytic enzymes, causing skin blistering and pruritus. IgG autoantibodies against BP180 and BP230 have been demonstrated to induce BP-like symptoms in animal models, and disease activity is associated with serum levels of anti-BP180 autoantibodies. Schmidt E et al., Arch Dermatol. 2000; 136 (2): 174-178; Masmoudi W et al., Br J Dermatol. 2021; 184 (6): 1106-1112. Anti-BP180 autoantibodies mainly recognize the extracellular 16th non-collagenous domain of the molecule (NC16A).

At this time, no therapies are specifically licensed for the treatment of patients with BP. The current standard of care for BP is treatment with topical corticosteroids or OCS, either of which can be combined with conventional immunosuppressant therapy. Unfortunately, corticosteroid therapy in patients with BP typically causes comorbidities, which can be severe and even life-threatening, especially in elderly patients. As a result, patients with BP have a higher mortality rate than their peers, even when their disease is being treated. Alternative therapies targeting IgG (e.g., IV administration of immunoglobulin [IVIg], plasma exchange, or protein A immunoadsorption) or biologics (e.g., rituximab, omalizumab) have shown potential in small numbers of BP patients. However, the efficacy and safety of these treatments have not been assessed in registrational studies. Amagai M et al., J Dermatol Sci. 2017; 85 (2): 77-84; Kasperkiewicz M et al., J Am Acad Dermatol. 2014; 71 (5): 1018-1020; Jafari S M S et al., Front Immunol. 2019; 10:1919.

In BP, patients typically achieve control of disease activity (CDA), partial remission (PR), or complete remission (CR) only after receiving treatment for several weeks; sometimes even months of treatment is required before CR or PR is achieved. Moreover, patients with BP tend to relapse within a few months after achieving remission.

Assessment of BP disease status and efficacy of BP treatment can be measured using validated scales established by experts in the field, including:

(1) Investigator Global Assessment of Bullous Pemphigoid (IGA-BP)—the IGA-BP is a new assessment tool that was developed in 2021 by medical experts in the field of AIBDs, and it is currently being used (along with the BPDAI) in other clinical studies of therapies for BP. The IGA categorizes the severity of BP on an ordinal scale of 0 (clear) to 4 (severe). An IGA-BP score of 0 (clear) indicates no signs of active disease are present (no blisters, erythema, or erosions). An IGA-BP score of 1 (almost clear) indicates no blisters or erosions are present and very limited erythematous, eczematous, or urticarial lesions are present. An IGA-BP score of 2 (mild) indicates few (<10) blisters or erosions are present and/or few erythematous, eczematous, or urticarial lesions (involving less than 10% of the body surface area) are present and no large erosive area (>5 cm) or no large blister (>5 cm) is present. An IGA-BP score of 3 (moderate) indicates multiple (10 to 29) blisters or erosions are present and/or multiple erythematous, eczematous, or urticarial lesions (involving 10-30% of the body surface area) are present and/or at the most, 2 large erosive areas (>5 cm) or large blisters (>5 cm) are present. An IGA-BP score of 4 (severe) indicates extensive (≥30) blisters or erosions are present and/or extensive erythematous, eczematous, or urticarial lesions (involving >30% of the body surface area) are present and/or 3 or more large erosive areas (>5 cm) or large blisters (>5 cm) are present.

(2) Bullous Pemphigoid Disease Area Index (BPDAI)—the BPDAI is an internationally validated tool to objectively measure disease activity. The BPDAI differentiates scores for skin (erosions/blisters and urticaria/erythema) and mucous membrane activity in several anatomical locations. In addition, separate scores for damage (e.g., pigmentation) are recorded to account for healing lesions. The BPDAI categorizes the severity of BP on a numerical scale of 0 (clear) to 120 (maximally severe) for skin and mucosa.

(3) Itch Numerical Rating Scale (Itch NRS)—Pruritic symptoms of BP will be indicated by the participant on the Itch NRS, recording an average and a worst score for itch suffered within the past 24 hours. An Itch NRS score of 0 indicates no pruritus; <3 indicates mild pruritus; >3 to <7 indicates moderate pruritus; >7 to <9 indicates severe pruritus; and >9 indicates very severe pruritis.

(4) EuroQOL 5-Dimension 5-Level (EQ-5D-5L)—the EQ-5D-5L is a standardized measure of health status developed by the EuroQOL Group to provide a simple, generic measure of health for clinical and economic appraisal. The descriptive system comprises 5 dimensions (mobility, self-care, usual activities, pain/discomfort, and anxiety/depression). Each dimension now has 5 levels: no problems, slight problems, moderate problems, severe problems, and extreme problems.

(5) Dermatology Life Quality Index (DLQI)—the DLQI consists of 10 questions concerning the participant's perception of the impact of skin diseases on different aspects of their health-related QOL the previous week. The impact of each aspect on the QoL assessment is scored qualitatively, ranging from “not at all” to “very much.”

(6) Autoimmune Bullous Disease Quality of Life (ABQOL) questionnaire—the ABQOL was developed and validated for determining the impact of AIBDs and their therapies on the daily life of patients. It is a series of 17 questions concerning the participant's perceptions of how AIBD is affecting their daily lives, including comfort, hygiene, eating/drinking, appearance, social interactions, sexual activity, and employment.

### FcRn Antagonists

FcRn antagonists that are useful in the methods and uses provided herein can include any molecule that binds to and inhibits FcRn, including but not limited to, any anti-FcRn antibody, any anti-FcRn binding region, or any Fc domain or Fc region.

In some embodiments, the FcRn antagonists disclosed herein comprise two, three, or four FcRn binding regions, such as an Fc region. In some embodiments, the FcRn antagonists disclosed herein comprise one or more Fc regions in combination with one or more Fab regions.

Any Fc region can be altered to produce a variant Fc region for use in the methods disclosed herein. In general, an Fc region, or FcRn binding fragment thereof, is from a human immunoglobulin. It is understood, however, that the Fc region may be derived from an immunoglobulin of any other mammalian species, including for example, a camelid species, a rodent (e.g., a mouse, rat, rabbit, guinea pig) or non-human primate (e.g., chimpanzee, macaque) species. Moreover, the Fc region or portion thereof may be derived from any immunoglobulin class, including IgM, IgG, IgD, IgA, and IgE, and any immunoglobulin isotype, including IgG1, IgG2, IgG3, and IgG4. In an embodiment, the Fc region is an IgG Fc region (e.g., a human IgG region). In an embodiment, the Fc region is an IgG1 Fc region (e.g., a human IgG1 region). In an embodiment, the Fc region is a chimeric Fc region comprising portions of several different Fc regions. Suitable examples of chimeric Fc regions are set forth in US 2011/0243966A1, which is incorporated herein by reference in its entirety. A variety of Fc region gene sequences (e.g., human constant region gene sequences) are available in the form of publicly accessible deposits.

An Fc region can be further truncated or internally deleted to produce a minimal FcRn binding fragment thereof. The ability of an Fc-region fragment to bind to FcRn can be determined using any art recognized binding assay (e.g., ELISA).

To enhance the manufacturability of the FcRn antagonists disclosed herein, it is preferable that the constituent Fc regions do not comprise any non-disulfide bonded cysteine residues. Accordingly, in an embodiment, the Fc regions do not comprise a free cysteine residue.

Any Fc variant, or FcRn binding fragment thereof, that binds specifically to FcRn with increased affinity and reduced pH dependence relative to the native (i.e., wild-type) Fc region can be used in the methods disclosed herein. In an embodiment, the variant Fc region comprises amino acid alterations, substitutions, insertions, and/or deletions that confer the desired characteristics. In some embodiments, the FcRn antagonist comprises or consists of a variant Fc region, or FcRn binding fragment thereof, which binds to FcRn with a higher affinity at pH 5.5 as compared to a corresponding wild-type Fc region. In some embodiments, the FcRn antagonist comprises or consists of a variant Fc region, or FcRn binding fragment thereof, which bind to FcRn with a higher affinity at pH 6.0 and/or at pH 7.4 as compared to a corresponding wild-type Fc region. In some embodiments, the FcRn antagonist comprises or consists of a variant Fc region, or FcRn binding fragment thereof, which bind to FcRn with a higher affinity at both acidic and neutral pH.

In some embodiments, the variant Fc region is derived from the Fc region of any native immunoglobulin. In some embodiments, the native immunoglobulin is a human immunoglobulin. In some embodiments, the immunoglobulin is IgA, IgD, IgE, or IgG. In some embodiments, the immunoglobulin is IgG. In some embodiments, the immunoglobulin is human IgA, human IgD, human IgE, or human IgG. In some embodiments, the immunoglobulin is human IgG. In some embodiments, the IgG is IgG1, IgG2, IgG3, or IgG4. In some embodiments, the human IgG is human IgG1, human IgG2, human IgG3, or human IgG4. In some embodiments, the variant Fc region varies from the human IgG1 Fc region. In some embodiments, the human IgG1 Fc region comprises a G1m1(a), G1m2(x), G1m3(f), or G1m17(z) allotype.

In an embodiment, the variant Fc region, or FcRn binding fragment thereof consists of two Fc domains. In an embodiment, the FcRn antagonist is an Fc region comprising amino acids Y, T, E, K, F, and Y at EU positions 252, 254, 256, 433, 434, and 436, respectively.

In an embodiment, the variant Fc region comprises or consists of a first Fc domain and a second Fc domain which form a homodimer or heterodimer. In an embodiment, the first Fc domain and/or the second Fc domain comprise amino acids Y, T, E, K, and F at EU positions 252, 254, 256, 433, and 434, respectively. In an embodiment, the first Fc domain and/or the second Fc domain comprise amino acids Y, T, E, K, F, and Y at EU positions 252, 254, 256, 433, 434, and 436, respectively.

In some embodiments, the FcRn antagonists disclosed herein comprise or consist of at least one Fc domain, wherein the amino acid sequence of the at least one Fc domain comprises or consists of the amino acid sequence of SEQ ID NO:13, provided below in Table 1.

TABLE 1

| | SEQ |
|---|---|
| | ID |
| Amino Acid Sequence | NO: |
| X₁X₂X₃X₄X₅X₆PPCPAPELLGGPSVFLFPPKPKDTLYITREPEV | 13 |
| TCVVVDVSHEDPEVKFNWYVDGVEVHNAKTKPREEQYNSTYRV | |
| VSVLTVLHQDWLNGKEYKCKVSNKALPAPIEKTISKAKGQPRE | |
| PQVYTLPPSRDELTKNQVSLTCLVKGFYPSDIAVEWESNGQPE | |
| NNYKTTPPVLDSDGSFFLYSKLTVDKSRWQQGNVFSCSVMHEA | |
| LKFHYTQKSLSLSPX₇X₈ | |

wherein:
X₁ is D or absent;
X₂ is K or absent;
X₃ is T or absent;
X₄ is H or absent;
X₅ is T or absent;
X₆ is C or absent;
X₇ is G or absent;
X₈ is K or absent.

In some embodiments, the FcRn antagonists disclosed herein comprise or consist of a variant Fc region comprising or consisting of a dimer of a first Fc domain and a second Fc domain, wherein the amino acid sequence of the first and second Fc domain comprises or consists of the amino acid sequence of SEQ ID NO: 13.

In some embodiments, the FcRn antagonists disclosed herein comprise or consist of an amino acid sequence at least 70%, 75%, 80%, 85%, 90%, 95%, 96%, 97%, 98%, 99%, or 100% identical to the amino acid sequence of a variant Fc region comprising or consisting of a dimer of a first Fc domain and a second Fc domain, wherein the amino acid sequence of the first and second Fc domain comprises or consists of an amino acid sequence independently selected from the group consisting of SEQ ID NOs: 1-3 and 14-31. In some embodiments, the dimer is a heterodimer or a homodimer.

TABLE 2

| | SEQ |
|---|---|
| Amino Acid Sequence | ID NO: |
| DKTHTCPPCPAPELLGGPSVFLFPPKPKDTLYITREPEVTCVVVDVSHEDPE | 2 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPGK | |
| DKTHTCPPCPAPELLGGPSVFLFPPKPKDTLYITREPEVTCVVVDVSHEDPE | 3 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPG | |
| DKTHTCPPCPAPELLGGPSVFLFPPKPKDTLYITREPEVTCVVVDVSHEDPE | 14 |
| VKFNWYVDGVEVHNAKTKPREEQYNSTYRVVSVLTVLHQDWLNGKEYKC | |
| KVSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSP | |
| KTHTCPPCPAPELLGGPSVFLFPPKPKDTLYITREPEVTCVVVDVSHEDPEV | 15 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPGK | |
| KTHTCPPCPAPELLGGPSVFLFPPKPKDTLYITREPEVTCVVVDVSHEDPEV | 16 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPG | |
| KTHTCPPCPAPELLGGPSVFLFPPKPKDTLYITREPEVTCVVVDVSHEDPEV | 17 |
| KFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSP | |
| THTCPPCPAPELLGGPSVFLFPPKPKDTLYITREPEVTCVVVDVSHEDPEVKF | 18 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPGK | |
| THTCPPCPAPELLGGPSVFLFPPKPKDTLYITREPEVTCVVVDVSHEDPEVKF | 19 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSPG | |
| THTCPPCPAPELLGGPSVFLFPPKPKDTLYITREPEVTCVVVDVSHEDPEVKF | 20 |
| NWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSV | |
| MHEALKFHYTQKSLSLSP | |
| TCPPCPAPELLGGPSVFLFPPKPKDTLYITREPEVTCVVVDVSHEDPEVKEN | 21 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| TCPPCPAPELLGGPSVFLFPPKPKDTLYITREPEVTCVVVDVSHEDPEVKEN | 22 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| TCPPCPAPELLGGPSVFLFPPKPKDTLYITREPEVTCVVVDVSHEDPEVKFN | 23 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| PPCPAPELLGGPSVFLFPPKPKDTLYITREPEVTCVVVDVSHEDPEVKFNWY | 24 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPGK | |
| PPCPAPELLGGPSVFLFPPKPKDTLYITREPEVTCVVVDVSHEDPEVKFNWY | 25 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSPG | |
| PPCPAPELLGGPSVFLFPPKPKDTLYITREPEVTCVVVDVSHEDPEVKFNWY | 26 |
| VDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIAVE | |
| WESNGQPENNYKTTPPVLDSDGSFFLYSKLTVDKSRWQQGNVFSCSVMHE | |
| ALKFHYTQKSLSLSP | |
| HTCPPCPAPELLGGPSVFLFPPKPKDTLYITREPEVTCVVVDVSHEDPEVKFN | 27 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPGK | |
| HTCPPCPAPELLGGPSVFLFPPKPKDTLYITREPEVTCVVVDVSHEDPEVKFN | 28 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSPG | |
| HTCPPCPAPELLGGPSVFLFPPKPKDTLYITREPEVTCVVVDVSHEDPEVKFN | 29 |
| WYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVM | |
| HEALKFHYTQKSLSLSP | |
| CPPCPAPELLGGPSVFLFPPKPKDTLYITREPEVTCVVVDVSHEDPEVKFNW | 1 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPG | |
| CPPCPAPELLGGPSVFLFPPKPKDTLYITREPEVTCVVVDVSHEDPEVKFNW | 30 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSPGK | |
| CPPCPAPELLGGPSVFLFPPKPKDTLYITREPEVTCVVVDVSHEDPEVKFNW | 31 |
| YVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMH | |
| EALKFHYTQKSLSLSP | |

In an embodiment, the first Fc domain and/or the second Fc domain comprise an amino acid sequence independently selected from the group consisting of SEQ ID NO: 1, SEQ ID NO: 2, and SEQ ID NO: 3. In an embodiment, the first Fc domain and the second Fc domain comprise an amino acid sequence independently selected from the group consisting of SEQ ID NO: 1, SEQ ID NO: 2, and SEQ ID NO: 3.

In an embodiment, the first Fc domain and/or the second Fc domain comprise an amino acid sequence independently selected from the group consisting of SEQ ID NOs: 1, 2, 3, and 30. In an embodiment, the first Fc domain and the second Fc domain comprise an amino acid sequence independently selected from the group consisting of SEQ ID NOs: 1, 2, 3, and 30.

In some embodiments, the FcRn antagonist comprises a population of FcRn antagonist molecules. In some embodiments, an FcRn antagonist comprising a first Fc domain and a second Fc domain comprising an amino acid sequence independently selected from the group consisting of SEQ ID NOs: 1, 2, and 3 is the predominant FcRn antagonist molecule in the population of FcRn antagonist molecules. In some embodiments, an FcRn antagonist comprising a first Fc domain and a second Fc domain comprising an amino acid sequence independently selected from the group consisting of SEQ ID NOs: 1, 2, 3, and 30 is the predominant FcRn antagonist molecule in the population of FcRn antagonist molecules. In some embodiments, the predominant FcRn antagonist molecule makes up at least 50%, 55%, 60%, 65%, 70%, 75%, 80%, 85%, 90%, 95%, 96%, 97%, 98%, or 99% of the population of FcRn antagonist molecules.

In an embodiment, the amino acid sequence of the Fc domains of the variant Fc region comprises the amino acid sequence of SEQ ID NO: 1. In an embodiment, the amino acid sequence of the Fc domains of the variant Fc region consists of the amino acid sequence of SEQ ID NO: 1. In an embodiment, the amino acid sequence of the Fc domains of the variant Fc region comprises the amino acid sequence of SEQ ID NO: 2. In an embodiment, the amino acid sequence of the Fc domains of the variant Fc region consists of the amino acid sequence of SEQ ID NO: 2. In an embodiment, the amino acid sequence of the Fc domains of the variant Fc region comprises the amino acid sequence of SEQ ID NO: 3. In an embodiment, the amino acid sequence of the Fc domains of the variant Fc region consists of the amino acid sequence of SEQ ID NO: 3. In an embodiment, the amino acid sequence of the Fc domains of the variant Fc region comprises the amino acid sequence of SEQ ID NO: 30. In an embodiment, the amino acid sequence of the Fc domains of the variant Fc region consists of the amino acid sequence of SEQ ID NO: 30.

In an embodiment, the FcRn antagonist consists of a variant Fc region, wherein the variant Fc region comprises two Fc domains, wherein the amino acid sequence of each of the Fc domains is independently selected from SEQ ID NO: 1, SEQ ID NO: 2, or SEQ ID NO: 3. In an embodiment, the FcRn antagonist consists of a variant Fc region, wherein the variant Fc region comprises two Fc domains, wherein the amino acid sequence of each of the Fc domains is independently selected from SEQ ID NO: 1, SEQ ID NO: 2, SEQ ID NO: 3, and SEQ ID NO: 30.

In certain embodiments, the variant Fc region is a heterodimer, where the constituent Fc domains are different from each other. Methods of producing Fc heterodimers are known in the art (see e.g., U.S. Pat. No. 8,216,805, which is incorporated by reference herein in its entirety). In an embodiment, the FcRn antagonist consists of a variant Fc region, wherein the variant Fc region consists of two Fc domains which form a heterodimer, wherein the amino acid sequence of each of the Fc domains is independently selected from SEQ ID NO: 1, SEQ ID NO: 2, or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 1, and the amino acid sequence of the second Fc domain consists of or comprises the amino acid sequence of SEQ ID NO: 2 or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 2, and the amino acid sequence of the second Fc domain consists of or comprises the amino acid sequence of SEQ ID NO: 1 or SEQ ID NO: 3. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 3, and the amino acid sequence of the second Fc domain consists of or comprises the amino acid sequence of SEQ ID NO: 1 or SEQ ID NO: 2.

In an embodiment, the FcRn antagonist consists of a variant Fc region, wherein the variant Fc region consists of two Fc domains which form a heterodimer, wherein the amino acid sequence of each of the Fc domains is independently selected from SEQ ID NO: 1, SEQ ID NO: 2, SEQ ID NO: 3, or SEQ ID NO: 30. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 1, and the amino acid sequence of the second Fc domain consists of or comprises the amino acid sequence of SEQ ID NO: 2, SEQ ID NO: 3, or SEQ ID NO: 30. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 2, and the amino acid sequence of the second Fc domain consists of or comprises the amino acid sequence of SEQ ID NO: 1, SEQ ID NO: 3, or SEQ ID NO: 30. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 3, and the amino acid sequence of the second Fc domain consists of or comprises the amino acid sequence of SEQ ID NO: 1, SEQ ID NO: 2, or SEQ ID NO: 30. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 30, and the amino acid sequence of the second Fc domain consists of or comprises the amino acid sequence of SEQ ID NO: 1, SEQ ID NO: 2, or SEQ ID NO: 3.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 1.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 2.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 3.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 30.

In some embodiments, the FcRn antagonist comprises glycanation on one or both of the Fc domains. In some embodiments, the FcRn antagonist molecules comprise glycanation at EU position 297 on one or both of the Fc domains. In some embodiments, the glycanation comprises an N-glycan. In some embodiments, the N-glycan comprises a GOF N-glycan, G1F N-glycan, G2F N-glycan, or G0 N-glycan.

In some embodiments, FcRn antagonist comprises or consists of a population of FcRn antagonists, wherein at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, or at least 57% of the population of Fc domains of the FcRn antagonists comprise galactose. In some embodiments, the population comprises or consists of FcRn antagonists, wherein at least 88%, at least 89%, at least 90%, at least 91%, at least 92%, at least 93%, at least 94%, at least 95%, at least 96%, at least 97%, at least 98%, or at least 99% of the population of Fc domains of the FcRn antagonists comprise fucose.

In some embodiments, the FcRn antagonist lacks an amino acid at EU position 441 of one or both Fc domains. In some embodiments, the FcRn antagonist comprises glycine and lysine at EU positions 440 and 441, respectively. In some embodiments, the FcRn antagonist lacks amino acids at EU positions 440 and 441. In some embodiments, the FcRn antagonist comprises amidated proline at EU position 439. In some embodiments, the FcRn antagonist lacks amino acids at EU positions 440 and 441 and comprises amidated proline at EU position 439.

In some embodiments, the FcRn antagonist comprises aspartate, lysine, threonine, histidine, threonine, and cysteine at EU positions 221, 222, 223, 224, 225, and 226, respectively. In some embodiments, the FcRn antagonist lacks an amino acid at EU positions 221, and comprises lysine, threonine, histidine, threonine, and cysteine at EU positions 222, 223, 224, 225, and 226, respectively. In some embodiments, the FcRn antagonist lacks amino acids at EU positions 221 and 222, and comprises threonine, histidine, threonine, and cysteine at EU positions 223, 224, 225, and 226, respectively. In some embodiments, the FcRn antagonist lacks amino acids at EU positions 221-224, and comprises threonine and cysteine at EU positions 225 and 226, respectively. In some embodiments, the FcRn antagonist lacks amino acids at EU positions 221, 222, 223, 224, 225, and 226.

In some embodiments, the FcRn antagonist is a population of FcRn antagonist molecules. In some embodiments, the population of FcRn antagonist molecules comprises or consists of multiple subpopulations of FcRn antagonist molecules. In some embodiments, the population of FcRn antagonist molecules comprises or consists of 2, 3, 4, 5, 6, 7, 8, 9, 10, 11, 12, 13, 14, 15, 16, 17, 18, 19, or 20 subpopulations. In some embodiments, the population of FcRn antagonist molecules comprises or consists of 2, 3, 4, 5, 6, 7, 8, 9, 10, or 11 subpopulations.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 22, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 19, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 19, respectively, and wherein one asparagine residue in each FcRn antagonist molecule in the fifth subpopulation are deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 16, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one methionine residue or one tryptophan residue in each FcRn antagonist molecule in the eleventh subpopulation is oxidized.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 22, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 19, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 19, respectively, and wherein one asparagine residue in each FcRn antagonist molecule in the fifth subpopulation is deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 16, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein one methionine residue or one tryptophan residue in each FcRn antagonist molecule in the eleventh subpopulation is oxidized.

In some embodiments, the population of FcRn antagonist molecules comprises or consists of the first subpopulation combined with one of the second, third, fourth, fifth, sixth, seventh, eighth, ninth, tenth, or eleventh subpopulations. In some embodiments, the population of FcRn antagonist molecules comprises or consists of the first subpopulation combined with two of the second, third, fourth, fifth, sixth, seventh, eighth, ninth, tenth, or eleventh subpopulations. In some embodiments, the population of FcRn antagonist molecules comprises or consists of the first subpopulation combined with three of the second, third, fourth, fifth, sixth, seventh, eighth, ninth, tenth, or eleventh subpopulations. In some embodiments, the population of FcRn antagonist molecules comprises or consists of the first subpopulation combined with four of the second, third, fourth, fifth, sixth, seventh, eighth, ninth, tenth, or eleventh subpopulations. In some embodiments, the population of FcRn antagonist molecules comprises or consists of the first subpopulation combined with five of the second, third, fourth, fifth, sixth, seventh, eighth, ninth, tenth, or eleventh subpopulations. In some embodiments, the population of FcRn antagonist molecules comprises or consists of the first subpopulation combined with six of the second, third, fourth, fifth, sixth, seventh, eighth, ninth, tenth, or eleventh subpopulations. In some embodiments, the population of FcRn antagonist molecules comprises or consists of the first subpopulation combined with seven of the second, third, fourth, fifth, sixth, seventh, eighth, ninth, tenth, or eleventh subpopulations. In some embodiments, the population of FcRn antagonist molecules comprises or consists of the first subpopulation combined with eight of the second, third, fourth, fifth, sixth, seventh, eighth, ninth, tenth, or eleventh subpopulations. In some embodiments, the population of FcRn antagonist molecules comprises or consists of the first subpopulation combined with nine of the second, third, fourth, fifth, sixth, seventh, eighth, ninth, tenth, or eleventh subpopulations. In some embodiments, the population of FcRn antagonist molecules comprises or consists of the first subpopulation combined with all of the second, third, fourth, fifth, sixth, seventh, eighth, ninth, tenth, or eleventh subpopulations.

In some embodiments, the population comprises or consists of the first and second subpopulations. In some embodiments, the population comprises or consists of the first and third subpopulations. In some embodiments, the population comprises or consists of the first and fourth subpopulations. In some embodiments, the population comprises or consists of the first and fifth subpopulations. In some embodiments, the population comprises or consists of the first and sixth subpopulations. In some embodiments, the population comprises or consists of the first and seventh subpopulations. In some embodiments, the population comprises or consists of the first and eighth subpopulations. In some embodiments, the population comprises or consists of the first and ninth subpopulations. In some embodiments, the population comprises or consists of the first and tenth subpopulations. In some embodiments, the population comprises or consists of the first and eleventh subpopulations. In some embodiments, the populations listed above further comprise or consist of 1, 2, 3, 4, 5, 6, 7, 8, or 9 additional subpopulations. In some embodiments, these additional subpopulations are one or more of those described above.

In some embodiments, the population comprises or consists of the first and seventh, ninth, or eleventh subpopulations. In some embodiments, the population comprises or consists of the first, seventh, ninth, and eleventh subpopulations.

In some embodiments, the first subpopulation makes up at least 40%, at least 45%, at least 50%, at least 55%, at least 60%, at least 65%, at least 70%, at least 75%, at least 80%, at least 85%, or at least 90% of the population of FcRn antagonist molecules. In some embodiments, the first subpopulation makes up about 40%, about 45%, about 50%, about 55%, about 60%, about 65%, about 70%, about 75%, about 80%, about 85%, or about 90% of the population of FcRn antagonist molecules. In some embodiments, the first subpopulation makes up 40%, 45%, 50%, 55%, 60%, 65%, 70%, 75%, 80%, 85%, or 90% of the population of FcRn antagonist molecules. In some embodiments, the first subpopulation makes up 40%-90%, 50%-80%, or 55%-70% of the population of FcRn antagonist molecules. In some embodiments, the first subpopulation makes up 56.9%-68.3% or 59.5%-67.9% of the population of FcRn antagonist molecules.

In some embodiments, the second subpopulation makes up less than 3.0%, less than 2.5%, less than 2.0%, less than 1.5%, less than 1%, or less than 0.5% of the population of FcRn antagonist molecules. In some embodiments, the second subpopulation makes up about 3.0%, about 2.5%, about 2.0%, about 1.5%, about 1%, or about 0.5% of the population of FcRn antagonist molecules. In some embodiments, the second subpopulation makes up 3.0%, 2.5%, 2.0%, 1.5%, 1%, or 0.5% of the population of FcRn antagonist molecules. In some embodiments, the second subpopulation makes up 0.5%-3.0%, 1.0%-2.5%, or 1.0%-2.0% of the population of FcRn antagonist molecules. In some embodiments, the second subpopulation makes up 0.8%-2.0% or 0.8%-2.1% of the population of FcRn antagonist molecules.

In some embodiments, the third subpopulation makes up less than 3.0%, less than 2.5%, less than 2.0%, less than 1.5%, less than 1%, or less than 0.5% of the population of FcRn antagonist molecules. In some embodiments, the third subpopulation makes up about 3.0%, about 2.5%, about 2.0%, about 1.5%, about 1%, or about 0.5% of the population of FcRn antagonist molecules. In some embodiments, the third subpopulation makes up 3.0%, 2.5%, 2.0%, 1.5%, 1%, or 0.5% of the population of FcRn antagonist molecules. In some embodiments, the third subpopulation makes up 0.5%-3.0%, 1.0%-2.5%, or 1.0%-2.0% of the population of FcRn antagonist molecules. In some embodiments, the third subpopulation makes up 1.1%-2.1% or 1.0%-1.9% of the population of FcRn antagonist molecules.

In some embodiments, the fourth subpopulation makes up less than 5%, less than 4%, less than 3%, less than 2%, or less than 1% of the population of FcRn antagonist molecules. In some embodiments, the fourth subpopulation makes up about 5%, about 4%, about 3%, about 2%, or about 1% of the population of FcRn antagonist molecules. In some embodiments, the fourth subpopulation makes up 5%, 4%, 3%, 2%, or 1% of the population of FcRn antagonist molecules. In some embodiments, the fourth subpopulation makes up 1%-5%, 2%-4%, or 2%-3% of the population of FcRn antagonist molecules. In some embodiments, the fourth subpopulation makes up 2.1%-3.2% or 2.0%-3.1% of the population of FcRn antagonist molecules.

In some embodiments, the fifth subpopulation makes up less than 12%, less than 11%, less than 10%, less than 9%, less than 8%, less than 7%, less than 6%, or less than 5% of the population of FcRn antagonist molecules. In some embodiments, the fifth subpopulation makes up about 12%, about 11%, about 10%, about 9%, about 8%, about 7%, about 6%, or about 5% of the population of FcRn antagonist molecules. In some embodiments, the fifth subpopulation makes up 12%, 11%, 10%, 9%, 8%, 7%, 6%, or 5% of the population of FcRn antagonist molecules. In some embodiments, the fifth subpopulation makes up 5%-12%, 6%-10%, or 7%-8% of the population of FcRn antagonist molecules. In some embodiments, the fifth subpopulation makes up 6.8%-9.4% or 6.9%-8.7% of the population of FcRn antagonist molecules.

In some embodiments, the sixth subpopulation makes up less than 17%, less than 16%, less than 15%, less than 14%, less than 13%, less than 12%, less than 11%, less than 10%, less than 9%, less than 8%, less than 7%, or less than 6% of the population of FcRn antagonist molecules. In some embodiments, the sixth subpopulation makes up about 17%, about 16%, about 15%, about 14%, about 13%, about 12%, about 11%, about 10%, about 9%, about 8%, about 7%, or about 6% of the population of FcRn antagonist molecules. In some embodiments, the sixth subpopulation makes up 17%, 16%, 15%, 14%, 13%, 12%, 11%, 10%, 9%, 8%, 7%, or 6% of the population of FcRn antagonist molecules. In some embodiments, the sixth subpopulation makes up 7%-17%, 10%-15%, or 11%-12% of the population of FcRn antagonist molecules. In some embodiments, the sixth subpopulation makes up 7.0%-14.0% or 10.0%-14.4% of the population of FcRn antagonist molecules.

In some embodiments, the seventh subpopulation makes up less than 6.0%, less than 5.5%, less than 5.0%, less than 4.5%, less than 4.0%, less than 3.5%, less than 3.0%, less than 2.5%, less than 2.0%, less than 1.5%, less than 1%, or less than 0.5% of the population of FcRn antagonist molecules. In some embodiments, the seventh subpopulation makes up about 6.0%, about 5.5%, about 5.0%, about 4.5%, about 4.0%, about 3.5%, about 3.0%, about 2.5%, about 2.0%, about 1.5%, about 1%, or about 0.5% of the population of FcRn antagonist molecules. In some embodiments, the seventh subpopulation makes up 6.0%, 5.5%, 5.0%, 4.5%, 4.0%, 3.5%, 3.0%, 2.5%, 2.0%, 1.5%, 1%, or 0.5% of the population of FcRn antagonist molecules. In some embodiments, the seventh subpopulation makes up 0.5%-5.5%, 1.0%-3.0%, or 1.5%-2.5% of the population of FcRn antagonist molecules. In some embodiments, the seventh subpopulation makes up 1.5%-5.5% or 1.4%-4.9% of the population of FcRn antagonist molecules.

In some embodiments, the eighth subpopulation makes up less than 7.5%, less than 7.0%, less than 6.5%, less than 6.0%, less than 5.5%, less than 5.0%, less than 4.5%, less than 4.0%, less than 3.5%, less than 3.0%, or less than 2.5% of the population of FcRn antagonist molecules. In some embodiments, the eighth subpopulation makes up about 7.5%, about 7.0%, about 6.5%, about 6.0%, about 5.5%, about 5.0%, about 4.5%, about 4.0%, about 3.5%, about 3.0%, or about 2.5% of the population of FcRn antagonist molecules. In some embodiments, the eighth subpopulation makes up 7.5%, 7.0%, 6.5%, 6.0%, 5.5%, 5.0%, 4.5%, 4.0%, 3.5%, 3.0%, or 2.5% of the population of FcRn antagonist molecules. In some embodiments, the eighth subpopulation makes up 2.5%-7.5%, 3.0%-5.0%, or 3.5%-4.5% of the population of FcRn antagonist molecules. In some embodiments, the eighth subpopulation makes up 2.9%-7.4% or 3.0%-6.3% of the population of FcRn antagonist molecules.

In some embodiments, the ninth subpopulation makes up less than 3.5%, less than 3.0%, less than 2.5%, less than 2.0%, less than 1.5%, less than 1%, or less than 0.5% of the population of FcRn antagonist molecules. In some embodiments, the ninth subpopulation makes up about 3.5%, about 3.0%, about 2.5%, about 2.0%, about 1.5%, about 1%, or about 0.5% of the population of FcRn antagonist molecules. In some embodiments, the ninth subpopulation makes up 3.5%, 3.0%, 2.5%, 2.0%, 1.5%, 1%, or 0.5% of the population of FcRn antagonist molecules. In some embodiments, the ninth subpopulation makes up 0.5%-3.5%, 1.5%-2.0%, or 1.0%-1.5% of the population of FcRn antagonist molecules. In some embodiments, the ninth subpopulation makes up 0.4%-3.2% or 0.5%-2.6% of the population of FcRn antagonist molecules.

In some embodiments, the tenth subpopulation makes up less than 2.0%, less than 1.5%, less than 1%, or less than 0.5% of the population of FcRn antagonist molecules. In some embodiments, the tenth subpopulation makes up about 2.0%, about 1.5%, about 1%, or about 0.5% of the population of FcRn antagonist molecules. In some embodiments, the tenth subpopulation makes up 2.0%, 1.5%, 1%, or 0.5% of the population of FcRn antagonist molecules. In some embodiments, the tenth subpopulation makes up 0.5%-2.0%, 0.5%-1.5%, or 1.0%-1.5% of the population of FcRn antagonist molecules.

In some embodiments, the eleventh subpopulation makes up less than 2.0%, less than 1.5%, less than 1%, or less than 0.5% of the population of FcRn antagonist molecules. In some embodiments, the eleventh subpopulation makes up about 2.0%, about 1.5%, about 1%, or about 0.5% of the population of FcRn antagonist molecules. In some embodiments, the eleventh subpopulation makes up 2.0%, 1.5%, 1%, or 0.5% of the population of FcRn antagonist molecules. In some embodiments, the eleventh subpopulation makes up 0.5%-2.0%, 0.5%-1.5%, or 1.0%-1.5% of the population of FcRn antagonist molecules.

In some embodiments, the population of FcRn antagonist molecules comprises one or more of the FcRn antagonists described herein. In some embodiments, the FcRn antagonist is any of those described in U.S. Patent Application No. 63/383,599, filed on Nov. 14, 2022, incorporated herein by reference in its entirety. In some embodiments, the FcRn antagonist is a population of FcRn antagonists as described in U.S. Patent Application No. 63/383,599, filed on Nov. 14, 2022, incorporated herein by reference in its entirety.

In an embodiment, the FcRn antagonist is efgartigimod (CAS Registry No. 1821402-21-4). The term “efgartigimod” as used herein is interchangeable with “efgartigimod alfa.” In some embodiments, efgartigimod is efgartigimod alfa-fcab.

In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161).

In an embodiment, an antibody that binds specifically to FcRn and inhibits the binding of the Fc region of immunoglobulin to FcRn is nipocalimab, also known as M281. Nipocalimab is a full-length “Fc dead” IgG1 monoclonal antibody. Nipocalimab has been administered as an intravenous infusion in Phase 2 clinical trials for the treatment of myasthenia gravis (MG), warm autoimmune hemolytic anemia (WAIHA), and hemolytic disease of fetus and newborn (HDFN). Nipocalimab comprises the light chain (SEQ ID NO: 4) and heavy chain (SEQ ID NO: 5) sequences set forth in Table 3 below:

TABLE 3
Heavy chain and light chain sequences
of nipocalimab

| SEQ | |
|---|---|
| ID | |
| NO: | Amino Acid Sequence |
| 4 | QSALTQPASVSGSPGQSITISCTGTGSDVGSYNLVSWYQQHPGKAP |
| | KLMIYGDSERPSGVSNRFSGSKSGNTASLTISGLQAEDEADYYCSS |
| | YAGSGIYVFGTGTKVTVLGQPKAAPSVTLFPPSSEELQANKATLVC |
| | LISDFYPGAVTVAWKADSSPVKAGVETTTPSKQSNNKYAASSYLSL |
| | TPEQWKSHKSYSCQVTHEGSTVEKTVAPTECS |
| 5 | EVQLLESGGGLVQPGGSLRLSCAASGFTFSTYAMGWVRQAPGKGL |
| | EWVSSIGASGSQTRYADSVKGRFTISRDNSKNTLYLQMNSLRAEDT |
| | AVYYCARLAIGDSYWGQGTMVTVSSASTKGPSVFPLAPSSKSTSGG |
| | TAALGCLVKDYFPEPVTVSWNSGALTSGVHTFPAVLQSSGLYSLSS |
| | VVTVPSSSLGTQTYICNVNHKPSNTKVDKKVEPKSCDKTHTCPPCP |
| | APELLGGPSVFLFPPKPKDTLMISRTPEVTCVVVDVSHEDPEVKFN |
| | WYVDGVEVHNAKTKPREEQYASTYRVVSVLTVLHQDWLNGKEYK |
| | CKVSNKALPAPIEKTISKAKGQPREPQVYTLPPSREEMTKNQVSLT |
| | CLVKGFYPSDIAVEWESNGQPENNYKTTPPVLDSDGSFFLYSKLTV |
| | DKSRWQQGNVFSCSVMHEALHNHYTQKSLSLSPG |

In an embodiment, an antibody that binds specifically to FcRn and inhibits the binding of the Fc region of immunoglobulin to FcRn is rozanolixizumab, also known as UCB 7665. Rozanolixizumab is a full-length humanized IgG4 monoclonal antibody. Rozanolixizumab has been administered as a subcutaneous infusion in ongoing clinical trials for MG, immune thrombocytopenia (ITP), and chronic inflammatory demyelinating polyneuropathy (CIDP). Rozanolixizumab comprises the light chain (SEQ ID NO: 6) and heavy chain (SEQ ID NO: 7) sequences set forth in Table 4 below:

TABLE 4
Heavy chain and light chain sequences
of rozanolixizumab

| SEQ | |
|---|---|
| ID | |
| NO: | Amino Acid Sequence |
| 6 | DIQMTQSPSSLSASVGDRVTITCKSSQSLVGASGKTYLYWLFQKPG |
| | KAPKRLIYLVSTLDSGIPSRFSGSGSGTEFTLTISSLQPEDFATYY |
| | CLQGTHFPHTFGQGTKLEIKRTVAAPSVFIFPPSDEQLKSGTASVV |
| | CLLNNFYPREAKVQWKVDNALQSGNSQESVTEQDSKDSTYSLSSTL |
| | TLSKADYEKHKVYACEVTHQGLSSPVTKSFNRGEC |
| 7 | EVPLVESGGGLVQPGGSLRLSCAVSGFTFSNYGMVWVRQAPGKGL |
| | EWVAYIDSDGDNTYYRDSVKGRFTISRDNAKSSLYLQMNSLRAEDT |
| | AVYYCTTGIVRPFLYWGQGTLVTVSSASTKGPSVFPLAPCSRSTSE |
| | STAALGCLVKDYFPEPVTVSWNSGALTSGVHTFPAVLQSSGLYSLS |
| | SVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMHEALHNHYTQKSLSLSLGK |

In an embodiment, an antibody that binds specifically to FcRn and inhibits the binding of the Fc region of immunoglobulin to FcRn is orilanolimab, also known as SYNT001. Orilanolimab is another full-length humanized IgG4 monoclonal antibody. Orilanolimab has been administered as an intravenous infusion in Phase 2 clinical trials for treatment of WAIHA. Orilanolimab comprises the light chain (SEQ ID NO: 8) and heavy chain (SEQ ID NO: 9) sequences set forth in Table 5 below:

TABLE 5
Heavy chain and light chain sequences
of orilanolimab

| SEQ | |
|---|---|
| ID | |
| NO: | Amino Acid Sequence |
| 8 | DIQMTQSPSSLSASVGDRVTITCKASDHINNWLAWYQQKPGQAPRL |
| | LISGATSLETGVPSRFSGSGTGKDYTLTISSLQPEDFATYYCQQYW |
| | STPYTFGGGTKVEIKRTVAAPSVFIFPPSDEQLKSGTASVVCLLNN |
| | FYPREAKVQWKVDNALQSGNSQESVTEQDSKDSTYSLSSTLTLSKA |
| | DYEKHKVYACEVTHQGLSSPVTKSFNRGEC |
| 9 | QVQLVQSGAELKKPGASVKLSCKASGYTFTSYGISWVKQATGQGLE |
| | WIGEIYPRSGNTYYNEKFKGRATLTADKSTSTAYMELRSLRSEDSA |
| | VYFCARSTTVRPPGIWGTGTTVTVSSASTKGPSVFPLAPCSRSTSE |
| | STAALGCLVKDYFPEPVTVSWNSGALTSGVHTFPAVLQSSGLYSLS |
| | SVVTVPSSSLGTKTYTCNVDHKPSNTKVDKRVESKYGPPCPPCPAP |
| | EFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCKV |
| | SNKGLPSSIEKTISKAKGQPREPQVYTLPPSQEEMTKNQVSLTCLV |
| | KGFYPSDIAVEWESNGQPENNYKTTPPVLDSDGSFFLYSRLTVDKS |
| | RWQEGNVFSCSVMHEALHNHYTQKSLSLSLG |

In an embodiment, an antibody that binds specifically to FcRn and inhibits the binding of the Fc region of immunoglobulin to FcRn is batoclimab, also known as IMVT1401/RVT1401/HBM9161. Batoclimab is another full-length “Fc dead” IgG1 monoclonal antibody. Batoclimab has been administered as a subcutaneous injection in ongoing Phase 2 clinical trials for treatment of MG and Graves' ophthalmopathy. Batoclimab comprises the light chain (SEQ ID NO: 10) and heavy chain (SEQ ID NO: 11) sequences set forth in Table 6 below:

TABLE 6
Heavy chain and light chain sequences
of batoclimab

| SEQ | |
|---|---|
| ID | |
| NO: | Amino Acid Sequence |
| 10 | SYVLTQSPSVSVAPGQTARITCGGNNIGSKSVHWYQQKPGQAPVLV |
| | VYDDSDRPSGIPERFSASNSGNTATLTISRVEAGDEADYYCQVWDS |
| | SSDHVVFGGGTKLTVLGQPKAAPSVTLFPPSSEELQANKATLVCLI |
| | SDFYPGAVTVAWKADSSPVKAGVETTTPSKQSNNKYAASSYLSLT |
| | PEQWKSHRSYSCQVTHEGSTVEKTVAPTECS |
| 11 | QLLLQESGPGLVKPSETLSLTCTVSGGSLSSSFSYWVWIRQPPGKG |
| | LEWIGTIYYSGNTYYNPSLKSRLTISVDTSKNHFSLKLSSVTAADT |
| | AVYYCARRAGILTGYLDSWGQGTLVTVSSASTKGPSVFPLAPSSKS |
| | TSGGTAALGCLVKDYFPEPVTVSWNSGALTSGVHTFPAVLQSSGLY |
| | SLSSVVTVPSSSLGTQTYICNVNHKPSNTKVDKRVEPKSCDKTHTC |
| | PPCPAPEAAGGPSVFLFPPKPKDTLMISRTPEVTCVVVDVSHEDPE |
| | VKFNWYVDGVEVHNAKTKPREEQYNSTYRVVSVLTVLHQDWLNGK |
| | EYKCKVSNKALPAPIEKTISKAKGQPREPQVYTLPPSREEMTKNQV |
| | SLTCLVKGFYPSDIAVEWESNGQPENNYKTTPPVLDSDGSFFLYSK |
| | LTVDKSRWQQGNVFSCSVMHEALHNHYTQKSLSLSPG |

### Pharmaceutical Compositions

In an aspect, the instant disclosure provides pharmaceutical compositions comprising an FcRn antagonist for use in methods of treating bullous pemphigoid (BP). In certain embodiments, these compositions comprise or consist of a variant Fc region, or FcRn binding fragment thereof, that binds specifically to FcRn, particularly human FcRn, with increased affinity and reduced pH dependence relative to a native Fc region. In other embodiments, the FcRn antagonist composition is an antibody or antigen-binding fragment thereof that binds specifically to FcRn via its antigen binding domain and inhibits the binding of Fc region of immunoglobulin to FcRn. In general, these FcRn antagonists inhibit the binding of Fc-containing agents (e.g., antibodies and immunoadhesins) to FcRn in vivo, which results in an increased rate of degradation of the Fc-containing agents and, concomitantly, a reduced serum level of these agents.

In an embodiment, the FcRn antagonist is efgartigimod. Efgartigimod (ARGX-113) is a modified human immunoglobulin (Ig) gamma (IgG) 1-derived Fc of the za allotype that binds with nanomolar affinity to human FcRn. Efgartigimod encompasses the IgG1 Fc region (encompassing residues of SEQ ID NO: 2) and has been engineered using ABDEG™ technology to increase its affinity for FcRn at both physiological and acidic pH. Vaccaro C et al., Nat Biotechnol. 2005; 23 (10): 1283. See also U.S. Pat. No. 10,316,073, the contents of which is incorporated by reference herein in its entirety. The increased affinity for FcRn of efgartigimod at both acidic and physiological pH results in a blockage of FcRn-mediated recycling of IgGs.

Efgartigimod has a molecular weight of about 54 kDa, which is about one-third the molecular weight of full-length IgG (MW ca. 150 kDa). Thus, 10 mg efgartigimod is about 185 nmol, such that a dose of 10 mg efgartigimod/kg body weight corresponds to about 185 nmol efgartigimod/kg body weight, and a dose of 25 mg efgartigimod/kg of body weight corresponds to about 462.5 nmol efgartigimod/kg body weight. In contrast, a dose of 10 mg full-length IgG antibody/kg body weight corresponds to about 67 nmol/kg body weight. Furthermore, a 1000 mg fixed dose of efgartigimod corresponds to a fixed dose of about 18,500 nmol of efgartigimod while a 2000 mg fixed dose of efgartigimod corresponds to a fixed dose of about 37,000 nmol of efgartigimod.

Due to its increased affinity for FcRn at both acidic and neutral pH, efgartigimod blocks the FcRn/IgG complex from forming, which results in degradation of endogenous IgGs, including autoantibodies that cause IgG-mediated autoimmune diseases. This blocking of FcRn by efgartigimod results in a rapid and profound reduction in autoantibody levels, which underlies the therapeutic strategy for the treatment of autoimmune indications where IgG autoantibodies are expected to have a central role in the disease pathology, e.g., conditions such as BP.

Efgartigimod is a prescription medicine for myasthenia gravis, which is approved in the United States and in Europe for the treatment of adults with generalized myasthenia gravis (gMG) who are anti-acetylcholine receptor (AChR) antibody positive, and in Japan for the treatment of adults with gMG who do not have sufficient response to steroids or non-steroidal immunosuppressive therapies (ISTs). Efgartigimod is under development for both the intravenous (IV) and subcutaneous (SC) administration route in multiple indications. For SC administration, in certain embodiments efgartigimod may be administered alone. Alternatively, for SC administration, in certain embodiments efgartigimod may be administered co-formulated with hyaluronidase, for example, in particular, rHuPH20. The co-formulated material will allow dosing of higher volumes.

rHuPH20 is the active ingredient of Halozyme's commercial product HYLENEX® recombinant (hyaluronidase human injection), referred to as HYLENEX®, which was approved by FDA for marketed use in the U.S. in December 2005. HYLENEX® is a tissue permeability modifier indicated as an adjuvant in SC fluid administration for achieving hydration, to increase the dispersion and absorption of other injected drugs, and in SC urography, for improving resorption of radiopaque agents.

rHuPH20 is a recombinant enzyme human hyaluronidase produced by genetically engineered Chinese hamster ovary (CHO) cells containing a deoxyribonucleic plasmid encoding a soluble fragment of human hyaluronidase (posterior head protein 20 [PH20]).

The HZ202 rHuPH20 DS is currently registered in HYLENEX® and other biologic drug products co-formulated with rHuPH20 DS. As such, in certain embodiments HZ202 rHuPH20 DS is used in the efgartigimod/rHuPH20 co-formulated product for SC administration (i.e., efgartigimod PH20 SC).

SC injection volumes are typically limited to 2.5 mL due to concerns regarding injection pain associated with larger volumes. It has been demonstrated that rHuPH20 offers a solution to the volume limitation associated with fast SC injections. rHuPH20 acts locally and transiently to depolymerize hyaluronan, a gel-like substance found in the subcutaneous layer of the skin. This results in decreased resistance to fluid flow and may increase dispersion and absorption of injected medicines and fluids, allowing for a larger volume to be injected with limited swelling or pain. It has been shown that rHuPH20 allows for the fast absorption of a relatively large volume (10 mL) when administered SC. Shpilberg O et al., Br J Cancer. 2013; 109 (6): 1556-1561. Very little injection site swelling was observed when 10 mL of IgG solution was administered SC using rHuPH20 at 2000 U/mL, whereas a large injection site swelling was observed when 10 mL of IgG solution was injected without rHuPH20. Shpilberg O et al., Br J Cancer. 2013; 109 (6): 1556-1561.

rHuPH20 is transiently acting and is not systematically absorbed. It has been demonstrated to exert no long-term local effects. rHuPH20 has a half-life in the skin of less than 30 minutes. Hyaluronan levels in subcutaneous tissues return to normal within 24 to 48 hours because of the rapid natural turnover of hyaluronan.

rHuPH20 is approved for SC administration in co-formulations with other active ingredients (RITUXAN HYCELA™/MABTHERA™ SC [rituximab] for Non-Hodgkin's lymphoma (NHL) and chronic lymphocytic leukemia (CLL) and HERCEPTIN HYLECTA™/HERCEPTIN™ SC [trastuzumab]) in the U.S. and Europe with an enzyme concentration of 2000 U/mL and an injectable volume that ranges from 5 to 13.4 mL.

In some embodiments, the pharmaceutical formulation comprises an FcRn antagonist in an amount from about 20 mg to about 20,000 mg. In some embodiments, the pharmaceutical formulation comprises an FcRn antagonist in an amount from about 200 mg to about 20,000 mg. In some embodiments, the pharmaceutical formulation comprises an FcRn antagonist in an amount from about 300 mg to about 6000 mg. In some embodiments, the pharmaceutical formulation comprises an FcRn antagonist in an amount from about 750 mg to about 3000 mg. In some embodiments, the pharmaceutical formulation comprises an FcRn antagonist in an amount from about 1000 mg to about 2500 mg. In some embodiments, the pharmaceutical formulation comprises an FcRn antagonist in an amount from about 1000 mg to about 2000 mg.

In some embodiments, the pharmaceutical formulation comprises an FcRn antagonist in an amount from 20 mg to 20,000 mg. In some embodiments, the pharmaceutical formulation comprises an FcRn antagonist in an amount from 200 mg to 20,000 mg. In some embodiments, the pharmaceutical formulation comprises an FcRn antagonist in an amount from 300 mg to 6000 mg. In some embodiments, the pharmaceutical formulation comprises an FcRn antagonist in an amount from 750 mg to 3000 mg. In some embodiments, the pharmaceutical formulation comprises an FcRn antagonist in an amount from 1000 mg to 2500 mg. In some embodiments, the pharmaceutical formulation comprises an FcRn antagonist in an amount from 1000 mg to 2000 mg.

In some embodiments, the pharmaceutical formulation comprises about 1000 mg or about 2000 mg of an FcRn antagonist. In some embodiments, the pharmaceutical formulation comprises 1000 mg or 2000 mg of an FcRn antagonist. In some embodiments, the FcRn antagonist is efgartigimod.

In some embodiments, the pharmaceutical formulation comprises efgartigimod in an amount from about 800 mg to about 1200 mg. In some embodiments, the pharmaceutical formulation comprises about 1000 mg efgartigimod. In some embodiments, the pharmaceutical formulation comprises 1000 mg efgartigimod.

In some embodiments, the pharmaceutical formulation comprises from about 10 mg/mL to about 200 mg/mL efgartigimod. In some embodiments, the pharmaceutical formulation comprises from 10 mg/mL to 200 mg/mL efgartigimod.

In some embodiments, the pharmaceutical formulation comprises about 20 mg/mL efgartigimod. In some embodiments, the pharmaceutical formulation comprises 20 mg/ml efgartigimod.

In some embodiments, the pharmaceutical formulation comprises about 180 mg/mL efgartigimod. In some embodiments, the pharmaceutical formulation comprises 180 mg/mL efgartigimod.

In some embodiments, the pharmaceutical formulation further comprises hyaluronidase. In some embodiments, the hyaluronidase is recombinant human hyaluronidase PH20 (rHuPH20).

The hyaluronidase can be present in the pharmaceutical formulation in any suitable amount. In an embodiment, the amount of hyaluronidase is from about 1000 U/mL to about 3000 U/mL. In an embodiment, the amount of hyaluronidase is about 1000 U/mL, about 1500 U/mL, about 2000 U/mL, about 2500 U/mL, or about 3000 U/mL. In an embodiment, the amount of hyaluronidase is 2000 U/mL.

In some embodiments, the rHuPH20 is present in the pharmaceutical formulation in an amount of about 11,000 U or about 22,000 U. In some embodiments, the rHuPH20 is present in the pharmaceutical formulation in an amount of 11,000 U or 22,000 U.

In any of the above embodiments, the pharmaceutical formulation may be a unit dosage form.

In an embodiment, the unit dosage form comprises the FcRn antagonist as a dry formulation for dissolution such as a lyophilized powder, freeze-dried powder, or water-free concentrate. In an embodiment, the dry formulation is comprised in a hermetically sealed container such as a vial, an ampoule, or a sachet.

In an embodiment, the unit dosage form comprises the FcRn antagonist as a liquid formulation, e.g., injection or infusion solution. In an embodiment, the liquid formulation is comprised in a hermetically sealed container such as a vial, a sachet, a pre-filled syringe, a pre-filled autoinjector, or a cartridge for a reusable syringe or applicator.

In an embodiment, the unit dosage per vial may contain 0.5 mL, 1 mL, 2 mL, 3 mL, 4 mL, 5 mL, 6 mL, 7 mL, 8 mL, 9 mL, 10 mL, 15 mL, or 20 mL of an FcRn antagonist ranging from about 500 to about 2500 mg or from about 1000 mg to about 2000 mg. In an embodiment, these preparations can be adjusted to a desired concentration by adding a sterile diluent to each vial.

The formulations disclosed herein include bulk drug compositions useful in the manufacture of pharmaceutical compositions (e.g., compositions that are suitable for administration to a subject or patient) which can be used in the preparation of unit dosage forms. In an embodiment, a composition of the invention is a pharmaceutical composition. Such compositions comprise a prophylactically or therapeutically effective amount of one or more prophylactic or therapeutic agents (e.g., an FcRn antagonist of the invention or other prophylactic or therapeutic agent), and a pharmaceutically acceptable carrier. In an embodiment, the pharmaceutical compositions are formulated to be suitable for intravenous administration to a subject. In an embodiment, the pharmaceutical compositions are formulated to be suitable for subcutaneous administration to a subject.

### Methods

In an aspect, methods for treating bullous pemphigoid (BP) using an FcRn antagonist are provided. In an aspect, provided herein is a method for treating BP in a subject in need thereof, the method comprising administering to the subject an effective amount of a human neonatal Fc receptor (FcRn) antagonist.

In some embodiments, the BP is mild BP, moderate BP, or severe BP. In some embodiments, the BP is moderate BP or severe BP. In some embodiments, the BP is moderate-to-severe BP. In certain embodiments, the FcRn antagonist is efgartigimod. An important goal and feature of the methods disclosed herein is the reduction or even the elimination of the use of potentially toxic agents such as corticosteroids (e.g., prednisone) in the treatment of BP. Another important goal and feature of the methods disclosed herein is rapid onset of disease control. Yet another important goal and feature of the methods disclosed herein is achievement of long-lasting complete remission on minimal or no treatment, preferably without the use of potentially toxic agents such as corticosteroids (e.g., prednisone). Effective treatment of BP using an FcRn antagonist may include at least one of the elements of the group consisting of: reduction of skin blistering, reduction of pruritis, QOL (Quality of Life) improvement, safety and/or tolerability of the FcRn antagonist, and steroid sparing effect.

In certain embodiments, severity of BP may be characterized by the Investigator Global Assessment of Bullous Pemphigoid (IGA-BP). In certain embodiments, the BP may be characterized as severe BP (e.g., IGA-BP score of 4). In certain embodiments, the BP may be characterized as moderate BP (e.g., IGA-BP score of 3). In certain embodiments, the BP may be characterized as mild BP (e.g., IGA-BP score of 2). In certain embodiments, the BP may be characterized as almost clear (e.g., IGA-BP score of 1). In certain embodiments, the BP may be characterized as clear (e.g., IGA-BP score of 0). In certain embodiments, the BP may be characterized as moderate-to-severe BP (e.g., IGA-BP score of 3 or 4). In certain embodiments, an IGA-BP score of 0 or 1 is indicative of effective treatment of BP. In certain embodiments, an IGA-BP score of 0 is indicative of effective treatment of BP.

In some embodiments, the BP is newly diagnosed BP, relapsing BP, or refractory BP. Relapsing BP refers to the occurrence of any of the following in a patient who has achieved control of disease activity (CDA): the appearance of 3 or more new lesions (such as blisters, eczematous lesions, or urticarial plaques) in 1 month (4 weeks); the appearance of at least 1 large (>10 cm diameter) eczematous lesion that does not heal within 1 week; the appearance of urticarial plaques that do not heal within 1 week; the extension/expansion of established lesions; and/or new or increased intensity of daily pruritic symptoms. Refractory BP refers to BP that is not controlled on current therapy. In some embodiments, refractory BP refers to BP that is not controlled on corticosteroids. In some embodiments, refractory BP refers to BP that is not controlled on immunosuppressants. In some embodiments, refractory BP refers to BP that is not controlled on corticosteroids and immunosuppressants. In some embodiments, refractory BP refers to failure to reach disease control with full therapeutic dose of systemic treatments, e.g., prednisone 1.5 mg/kg/day for 3 weeks.

In some embodiments, the FcRn antagonist is administered at a fixed dose of about 20 mg to about 20,000 mg. In some embodiments, the FcRn antagonist is administered at a fixed dose of about 200 mg to about 20,000 mg. In some embodiments, the FcRn antagonist is administered at a fixed dose of about 300 mg to about 6000 mg. In some embodiments, the FcRn antagonist is administered at a fixed dose of about 750 mg to about 3000 mg. In some embodiments, the FcRn antagonist is administered at a fixed dose of about 1000 mg to about 2500 mg. In some embodiments, the FcRn antagonist is administered at a fixed dose of about 1000 mg to about 2000 mg. In some embodiments, the FcRn antagonist is efgartigimod.

In some embodiments, the FcRn antagonist is administered at a fixed dose of 20 mg to 20,000 mg. In some embodiments, the FcRn antagonist is administered at a fixed dose of 200 mg to 20,000 mg. In some embodiments, the FcRn antagonist is administered at a fixed dose of 300 mg to 6000 mg. In some embodiments, the FcRn antagonist is administered at a fixed dose of 750 mg to 3000 mg. In some embodiments, the FcRn antagonist is administered at a fixed dose of 1000 mg to 2500 mg. In some embodiments, the FcRn antagonist is administered at a fixed dose of 1000 mg to 2000 mg. In some embodiments, the FcRn antagonist is efgartigimod.

In some embodiments, the FcRn antagonist is administered at a fixed dose of about 20 mg, about 50 mg, about 100 mg, about 200 mg, about 250 mg, about 300 mg, about 500 mg, about 750 mg, about 1000 mg, about 1500 mg, about 2000 mg, about 2500 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg. In some embodiments, the FcRn antagonist is efgartigimod.

In some embodiments, the FcRn antagonist is administered at a fixed dose of 20 mg, 50 mg, 100 mg, 200 mg, 250 mg, 300 mg, 500 mg, 750 mg, 1000 mg, 1500 mg, 2000 mg, 2500 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg. In some embodiments, the FcRn antagonist is efgartigimod.

In some embodiments, the FcRn antagonist is administered at a dose of about 0.2 mg/kg to about 200 mg/kg. In some embodiments, the FcRn antagonist is administered at a dose of about 2 mg/kg to about 200 mg/kg. In some embodiments, the FcRn antagonist is administered at a dose of about 2 mg/kg to about 120 mg/kg. In some embodiments, the FcRn antagonist is administered at a dose of about 3 mg/kg to about 60 mg/kg. In some embodiments, the FcRn antagonist is administered at a dose of about 10 mg/kg to about 25 mg/kg. In some embodiments, the FcRn antagonist is efgartigimod.

In some embodiments, the FcRn antagonist is administered at a dose of 0.2 mg/kg to 200 mg/kg. In some embodiments, the FcRn antagonist is administered at a dose of about 2 mg/kg to about 200 mg/kg. In some embodiments, the FcRn antagonist is administered at a dose of 2 mg/kg to 120 mg/kg. In some embodiments, the FcRn antagonist is administered at a dose of 3 mg/kg to 60 mg/kg. In some embodiments, the FcRn antagonist is administered at a dose of 10 mg/kg to 25 mg/kg. In some embodiments, the FcRn antagonist is efgartigimod.

In some embodiments, the FcRn antagonist is administered at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously. In some embodiments, the FcRn antagonist is administered intravenously once weekly, once every two weeks, once every three weeks, once every four weeks, once monthly, or once every six weeks. In some embodiments, the FcRn antagonist is efgartigimod.

In some embodiments, the FcRn antagonist is administered intravenously once weekly or once every two weeks at a dose of about 0.2 mg/kg to about 200 mg/kg. In some embodiments, the FcRn antagonist is administered intravenously once weekly or once every two weeks at a dose of about 2 mg/kg to about 200 mg/kg. In some embodiments, the FcRn antagonist is administered intravenously once weekly or once every two weeks at a dose of about 2 mg/kg to about 120 mg/kg. In some embodiments, the FcRn antagonist is administered intravenously once weekly or once every two weeks at a dose of about 3 mg/kg to about 60 mg/kg. In some embodiments, the FcRn antagonist is administered intravenously once weekly or once every two weeks at a dose of about 10 mg/kg to about 25 mg/kg. In some embodiments, the FcRn antagonist is efgartigimod.

In some embodiments, the FcRn antagonist is administered intravenously once weekly or once every two weeks at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered intravenously once weekly or once every two weeks at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously once weekly or once every two weeks at a dose of about 10 mg/kg to about 30 mg/kg. In some embodiments, the FcRn antagonist is administered intravenously once weekly or once every two weeks at a dose of about 10 mg/kg to about 25 mg/kg. In some embodiments, the FcRn antagonist is administered intravenously once weekly or once every two weeks at a dose of about 10 mg/kg. In some embodiments, the FcRn antagonist is administered intravenously once weekly or once every two weeks at a dose of about 15 mg/kg. In some embodiments, the FcRn antagonist is administered intravenously once weekly or once every two weeks at a dose of about 20 mg/kg. In some embodiments, the FcRn antagonist is administered intravenously once weekly or once every two weeks at a dose of about 25 mg/kg. In some embodiments, the FcRn antagonist is administered intravenously once weekly or once every two weeks at a dose of about 30 mg/kg. In some embodiments, the FcRn antagonist is administered intravenously once weekly or once every two weeks at a dose of 10 mg/kg to 30 mg/kg. In some embodiments, the FcRn antagonist is administered intravenously once weekly or once every two weeks at a dose of 10 mg/kg to 25 mg/kg. In some embodiments, the FcRn antagonist is administered intravenously once weekly or once every two weeks at a dose of 10 mg/kg. In some embodiments, the FcRn antagonist is administered intravenously once weekly or once every two weeks at a dose of 15 mg/kg. In some embodiments, the FcRn antagonist is administered intravenously once weekly or once every two weeks at a dose of 20 mg/kg. In some embodiments, the FcRn antagonist is administered intravenously once weekly or once every two weeks at a dose of 25 mg/kg. In some embodiments, the FcRn antagonist is administered intravenously once weekly or once every two weeks at a dose of 30 mg/kg. In some embodiments, the FcRn antagonist is efgartigimod.

In some embodiments, the FcRn antagonist is administered subcutaneously. In some embodiments, the FcRn antagonist is administered subcutaneously once weekly, once every two weeks, once every three weeks, once every four weeks, once monthly, or once every six weeks. In some embodiments, the FcRn antagonist is efgartigimod.

In some embodiments, FcRn antagonist is administered subcutaneously at a fixed dose of about 20 mg to about 20,000 mg. In some embodiments, FcRn antagonist is administered subcutaneously at a fixed dose of about 100 mg to about 10,000 mg once weekly, once every two weeks, once every three weeks, once every four weeks, once monthly, or once every six weeks. In some embodiments, the FcRn antagonist is administered subcutaneously at a fixed dose of 750 mg to 3000 mg once weekly, once every two weeks, once every three weeks, once every four weeks, once monthly, or once every six weeks. In some embodiments, the FcRn antagonist is administered subcutaneously at a fixed dose of 1000 mg to 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of about 20 mg, about 50 mg, about 100 mg, about 250 mg, about 500 mg, about 750 mg, about 1000 mg, about 1500 mg, about 2000 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of 20 mg, 50 mg, 100 mg, 250 mg, 500 mg, 750 mg, 1000 mg, 1500 mg, 2000 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg once weekly, once every two weeks, once every three weeks, once every four weeks, once monthly, or once every six weeks. In some embodiments, the FcRn antagonist is administered subcutaneously at a fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously once weekly or once every two weeks at a fixed dose of about 750 mg to about 3000 mg. In some embodiments, the FcRn antagonist is administered subcutaneously once weekly or once every two weeks at a fixed dose of about 1000 mg to about 2000 mg. In some embodiments, the FcRn antagonist is administered subcutaneously at a fixed dose of about 1000 mg or about 2000 mg once weekly or once every two weeks. In some embodiments, the FcRn antagonist is efgartigimod.

In some embodiments, the FcRn antagonist is administered subcutaneously once weekly or once every two weeks at a fixed dose of 750 mg to 3000 mg. In some embodiments, the FcRn antagonist is administered subcutaneously once weekly or once every two weeks at a fixed dose of 1000 mg to 2000 mg. In some embodiments, the FcRn antagonist is administered subcutaneously at a fixed dose of 1000 mg or 2000 mg once weekly or once every two weeks. In some embodiments, the FcRn antagonist is efgartigimod.

In some embodiments, the FcRn antagonist is first administered subcutaneously at a fixed dose of about 1000 mg twice on the same day. In some embodiments, the FcRn antagonist is first administered subcutaneously at a fixed dose of 1000 mg twice on the same day. In some embodiments, the FcRn antagonist is efgartigimod.

In some embodiments, the FcRn antagonist is administered subcutaneously at a fixed dose of about 2000 mg for the first two administrations and is subsequently administered subcutaneously at a fixed dose of about 1000 mg. In some embodiments, the FcRn antagonist is administered subcutaneously at a fixed dose of 2000 mg for the first two administrations and is subsequently administered subcutaneously at a fixed dose of 1000 mg. In some embodiments, the first two administrations of the FcRn antagonist are subcutaneous administrations at a fixed dose of about 1000 mg administered twice on the same day. In some embodiments, the first two administrations of the FcRn antagonist are subcutaneous administrations at a fixed dose of 1000 mg administered twice on the same day. In some embodiments, the FcRn antagonist is efgartigimod.

In some embodiments, the FcRn antagonist is administered subcutaneously once weekly at a fixed dose of about 750 mg to about 1750 mg. In some embodiments, the FcRn antagonist is administered subcutaneously once weekly at a fixed dose of about 800 mg to about 1200 mg. In some embodiments, the FcRn antagonist is administered subcutaneously once weekly at a fixed dose of about 750 mg. In some embodiments, the FcRn antagonist is administered subcutaneously once weekly at a fixed dose of about 800 mg. In some embodiments, the FcRn antagonist is administered subcutaneously once weekly at a fixed dose of about 1000 mg. In some embodiments, the FcRn antagonist is administered subcutaneously once weekly at a fixed dose of about 1200 mg. In some embodiments, the FcRn antagonist is administered subcutaneously once weekly at a fixed dose of about 1250 mg. In some embodiments, the FcRn antagonist is administered subcutaneously once weekly at a fixed dose of about 1500 mg. In some embodiments, the FcRn antagonist is administered subcutaneously once weekly at a fixed dose of about 1750 mg. In some embodiments, the FcRn antagonist is efgartigimod.

In some embodiments, the FcRn antagonist is administered subcutaneously once weekly at a fixed dose of 750 mg to 1750 mg. In some embodiments, the FcRn antagonist is administered subcutaneously once weekly at a fixed dose of 800 mg to 1200 mg. In some embodiments, the FcRn antagonist is administered subcutaneously once weekly at a fixed dose of 750 mg. In some embodiments, the FcRn antagonist is administered subcutaneously once weekly at a fixed dose of 800 mg. In some embodiments, the FcRn antagonist is administered subcutaneously once weekly at a fixed dose of 1000 mg. In some embodiments, the FcRn antagonist is administered subcutaneously once weekly at a fixed dose of 1200 mg. In some embodiments, the FcRn antagonist is administered subcutaneously once weekly at a fixed dose of 1250 mg. In some embodiments, the FcRn antagonist is administered subcutaneously once weekly at a fixed dose of 1500 mg. In some embodiments, the FcRn antagonist is administered subcutaneously once weekly at a fixed dose of 1750 mg. In some embodiments, the FcRn antagonist is efgartigimod.

In some embodiments, the FcRn antagonist is administered subcutaneously once weekly at a dose of about 10 mg/kg to about 25 mg/kg. In some embodiments, the FcRn antagonist is administered subcutaneously once weekly at a dose of about 10 mg/kg. In some embodiments, the FcRn antagonist is administered subcutaneously once weekly at a dose of about 15 mg/kg. In some embodiments, the FcRn antagonist is administered subcutaneously once weekly at a dose of about 20 mg/kg. In some embodiments, the FcRn antagonist is administered subcutaneously once weekly at a dose of about 25 mg/kg. In some embodiments, the FcRn antagonist is efgartigimod.

In some embodiments, the FcRn antagonist is administered subcutaneously once weekly at a dose of 10 mg/kg to 25 mg/kg. In some embodiments, the FcRn antagonist is administered subcutaneously once weekly at a dose of 10 mg/kg. In some embodiments, the FcRn antagonist is administered subcutaneously once weekly at a dose of 15 mg/kg. In some embodiments, the FcRn antagonist is administered subcutaneously once weekly at a dose of 20 mg/kg. In some embodiments, the FcRn antagonist is administered subcutaneously once weekly at a dose of 25 mg/kg. In some embodiments, the FcRn antagonist is efgartigimod.

In some embodiments, the FcRn antagonist is first administered intravenously and is subsequently administered subcutaneously. In some embodiments, the FcRn antagonist is first administered intravenously and is subsequently administered subcutaneously at fixed dose of 100 mg to 10,000 mg once weekly, once every two weeks, once every three weeks, once every four weeks, once monthly, or once every six weeks. In some embodiments, the FcRn antagonist is first administered intravenously and is subsequently administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, one or more doses of the FcRn antagonist are administered intravenously and subsequent doses of the FcRn antagonist are administered subcutaneously. In some embodiments, one or more doses of the FcRn antagonist are administered intravenously and subsequent doses of the FcRn antagonist are administered subcutaneously at fixed dose of 100 mg to 10,000 mg once weekly, once every two weeks, once every three weeks, once every four weeks, once monthly, or once every six weeks. In some embodiments, one or more doses of the FcRn antagonist are administered intravenously and subsequent doses of the FcRn antagonist are administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is administered for 6, 12, 26, 36, or 52 weeks or less. In some embodiments, the FcRn antagonist is administered for 36 weeks or less. In some embodiments, the FcRn antagonist is administered for 52 weeks or less. In some embodiments, the FcRn antagonist is administered for at least 6, 12, 26, 36, or 52 weeks. In some embodiments, the FcRn antagonist is administered for at least 36 weeks. In some embodiments, the FcRn antagonist is administered for at least 52 weeks.

In some embodiments, the FcRn antagonist is administered once weekly under disease control or complete remission.

In some embodiments, the FcRn antagonist is rozanolixizumab. In some embodiments, rozanolixizumab is administered subcutaneously or intravenously. In some embodiments, rozanolixizumab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, rozanolixizumab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is nipocalimab. In some embodiments, nipocalimab is administered subcutaneously or intravenously. In some embodiments, nipocalimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, nipocalimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is orilanolimab. In some embodiments, orilanolimab is administered subcutaneously or intravenously. In some embodiments, orilanolimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, orilanolimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered intravenously at a dose of about 30 mg/kg once weekly for three weeks and then at a dose of 10 mg/kg administered intravenously every other week.

In some embodiments, the FcRn antagonist is batoclimab. In some embodiments, batoclimab is administered subcutaneously or intravenously. In some embodiments, batoclimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, batoclimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In an embodiment, the method further comprises administering to the subject an effective amount of a corticosteroid and/or an immunosuppressive agent (such as methotrexate, mycophenolate mofetil (MMF), azathioprine, cyclophosphamide, doxycycline, and dapsone). In an embodiment, the effective amount of the corticosteroid is administered at a dose of 2.5 mg/day to 20 mg/day. In an embodiment, the effective amount of the corticosteroid is administered at a dose of 5 mg/day to 20 mg/day. In an embodiment, the effective amount of the corticosteroid is administered at a dose of 7.5 mg/day to 20 mg/day. In an embodiment, the effective amount of the corticosteroid is administered at a dose of 8 mg/day to 15 mg/day. In an embodiment, the effective amount of the corticosteroid is administered at a dose of 10 mg/day to 15 mg/day. In an embodiment, the effective amount of the corticosteroid is administered at a dose of about 20 mg/day. In an embodiment, the effective amount of the corticosteroid is administered at a dose of about 10 mg/day. In an embodiment, the effective amount of the corticosteroid is administered at a dose of about 7.5 mg/day. In an embodiment, the effective amount of the corticosteroid is administered at a dose of about 5 mg/day. In an embodiment, the effective amount of the corticosteroid is administered at a dose of about 2.5 mg/day. In an embodiment, the effective amount of the corticosteroid is administered at a dose of about 1 mg/kg/day. In an embodiment, the effective amount of the corticosteroid is administered at a dose of about 0.75 mg/kg/day. In an embodiment, the effective amount of the corticosteroid is administered at a dose of about 0.5 mg/kg/day. In an embodiment, the effective amount of the corticosteroid is administered at a dose of about 0.4 mg/kg/day. In an embodiment, the effective amount of the corticosteroid is administered at a dose of about 0.3 mg/kg/day. In an embodiment, the effective amount of the corticosteroid is administered at a dose of about 0.25 mg/kg/day. In an embodiment, the effective amount of the corticosteroid is administered at a dose of about 0.2 mg/kg/day. In an embodiment, the effective amount of the corticosteroid is administered at a dose of about 0.15 mg/kg/day. In an embodiment, the effective amount of the corticosteroid is administered at a dose of about 0.1 mg/kg/day.

The corticosteroid may be an oral corticosteroid, a topical corticosteroid or both an oral corticosteroid and a topical corticosteroid. In certain embodiments, the corticosteroid is an oral corticosteroid. Examples of oral corticosteroids include, but are not limited to, betamethasone, cortisone, dexamethasone, hydrocortisone, methylprednisolone, prednisone, prednisolone, and triamcinolone. In certain embodiments, the corticosteroid is a topical corticosteroid. Topical corticosteroids are well-known and include compounds of various potencies and formulations. These are generally formulated as ointments, creams, oils, lotions, shampoos, foams, and/or gels. Examples of commercially available topical corticosteroids include alclometasone dipropionate, amcinonide, betamethasone dipropionate, betamethasone valerate, clobetasol propionate, clobetasone butyrate, desonide, desoximetasone, diflucortolone valerate, diflorasone diacetate, fluocinolone acetonide, fluocinonide, flurandrenolide, fluticasone propionate, halcinonide, halobetasol propionate, hydrocortisone, hydrocortisone 17-butyrate, hydrocortisone acetate, hydrocortisone valerate, methylprednisolone aceponate, mometasone furoate, prednicarbate, and triamcinolone acetonide. Topical corticosteroids conveniently can be applied locally to affected areas or lesions in need of treatment, thereby limiting undesirable systemic effects of corticosteroid treatment.

In an embodiment, the corticosteroid dosing regimen is tapered in the subject during or after treatment with the FcRn antagonist. In an embodiment, tapering the corticosteroid regimen is lowering the dose or lowering the dosing frequency of the corticosteroid. In an embodiment, the dose of corticosteroid is reduced after CDA or remission (partial or complete) is achieved. In an embodiment, the dose of corticosteroid is reduced after CDA has been sustained for at least two weeks. In an embodiment, the dose of corticosteroid is reduced by following a tapering schedule. Examples of tapering schedules that may be used to reduce corticosteroid dosing are provided herein. One of skill in the art will appreciate that tapering schedules may vary and may be adapted depending upon multiple subject-specific factors, such as the initial corticosteroid dose, the appearance of new lesions or other BP symptoms, overall health of the subject, etc.

In some embodiments, following two weeks of sustained CDA, the daily dose of prednisone (or equivalent OCS) is decreased from 0.5 mg/kg/day to 0.3 mg/kg/day, from 0.3 mg/kg/day to 0.2 mg/kg/day, from 0.2 mg/kg/day to 0.15 mg/kg/day and from 0.15 mg/kg/day to 0.1 mg/kg/day. In some embodiments, each daily dose of prednisone (or equivalent OCS) is maintained for at least two weeks before the dose is further decreased. In some embodiments, the daily dose of prednisone (or equivalent OCS) is maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 2 weeks. In some embodiments, the daily dose of prednisone (or equivalent OCS) is maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 8 weeks. In some embodiments, each daily dose is maintained for two weeks provided that no new lesions appear.

In some embodiments, following two weeks of sustained disease control, the daily dose of prednisone (or equivalent OCS) is decreased from 0.75 mg/kg/day to 0.5 mg/kg/day, from 0.5 mg/kg/day to 0.3 mg/kg/day, from 0.3 mg/kg/day to 0.2 mg/kg/day, from 0.2 mg/kg/day to 0.15 mg/kg/day, and from 0.15 mg/kg/day to 0.1 mg/kg/day. In some embodiments, each daily dose of prednisone (or equivalent OCS) is maintained for at least two weeks before the dose is further decreased. In some embodiments, the daily dose of prednisone (or equivalent OCS) is maintained at 0.5 mg/kg/day for at least 2 weeks, then maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 2 weeks. In some embodiments, the daily dose of prednisone (or equivalent OCS) is maintained at 0.5 mg/kg/day for at least 2 weeks, then maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 8 weeks. In some embodiments, each daily dose is maintained for two weeks provided that no new lesions appear.

In some embodiments, following two weeks of sustained disease control, the daily dose of prednisone (or equivalent OCS) is decreased from 1 mg/kg/day to 0.75 mg/kg/day, from 0.75 mg/kg/day to 0.5 mg/kg/day, from 0.5 mg/kg/day to 0.3 mg/kg/day, from 0.3 mg/kg/day to 0.2 mg/kg/day, from 0.2 mg/kg/day to 0.15 mg/kg/day, and from 0.15 mg/kg/day to 0.1 mg/kg/day. In some embodiments, each daily dose of prednisone (or equivalent OCS) is maintained for at least two weeks before the dose is further decreased. In some embodiments, the daily dose of prednisone (or equivalent OCS) is maintained at 0.75 mg/kg/day for at least 2 weeks, then maintained at 0.5 mg/kg/day for at least 2 weeks, then maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 2 weeks. In some embodiments, the daily dose of prednisone (or equivalent OCS) is maintained at 0.75 mg/kg/day for at least 2 weeks, then maintained at 0.5 mg/kg/day for at least 2 weeks, then maintained at 0.3 mg/kg/day for at least 2 weeks, then maintained at 0.2 mg/kg/day for at least 2 weeks, then maintained at 0.15 mg/kg/day for at least 2 weeks, and then maintained at 0.1 mg/kg/day for at least 8 weeks. In some embodiments, each daily dose is maintained for two weeks provided that no new lesions appear.

After maintenance at 0.1 mg/kg/day for at least 2 weeks, the daily dose of prednisone (or equivalent OCS) may optionally be further reduced by 2.5 mg every two weeks for one or more two-week periods. In some embodiments, the daily dose of prednisone (or equivalent OCS) is maintained at 0.1 mg/kg/day for at least 8 weeks before it is further reduced by 2.5 mg every two weeks for one or more two-week periods. In some embodiments, the daily dose of prednisone (or equivalent OCS) is decreased by 2.5 mg every two weeks for one or more two-week periods until the daily dose of prednisone (or equivalent OCS) is zero (i.e., discontinuation of corticosteroid therapy). In some embodiments, each daily dose is maintained for two weeks provided that no new lesions appear.

Alternatively, after maintenance at 0.1 mg/kg/day for at least 2 weeks, the daily dose of prednisone (or equivalent OCS) may optionally be further reduced by 1 mg every week for one or more one-week periods. In some embodiments, the daily dose of prednisone (or equivalent OCS) is maintained at 0.1 mg/kg/day for at least 8 weeks before it is further reduced by 1 mg every week for one or more one-week periods. In some embodiments, the daily dose of prednisone (or equivalent OCS) is decreased by 1 mg every week for one or more one-week periods until the daily dose of prednisone (or equivalent OCS) is zero (i.e., discontinuation of corticosteroid therapy). In some embodiments, each daily dose is maintained for one week provided that no new lesions appear.

In some embodiments, the FcRn antagonist is administered in an induction phase and a maintenance phase. In certain embodiments, during the induction phase, the FcRn antagonist is administered once weekly or more frequently, e.g., twice a week or every other day. In certain embodiments, during the induction phase, the FcRn antagonist is administered less frequently than once weekly, e.g., once every other week. In certain embodiments, (i) during the induction phase the FcRn antagonist is administered once weekly concurrently with corticosteroid and/or immunosuppressant; and (ii) during the maintenance phase the FcRn antagonist dose is decreased and/or the FcRn antagonist dosing interval is lengthened, e.g., to once every two weeks, with or without concurrent corticosteroid and/or immunosuppressant. In some embodiments, the corticosteroid dose is decreased and/or the corticosteroid dosing interval is lengthened during the maintenance phase. In some embodiments, (i) during the induction phase the FcRn antagonist is administered once weekly at a dose of 1000 mg; and (ii) during the maintenance phase the FcRn antagonist is administered once every two weeks at a dose of 1000 mg.

In some embodiments, the FcRn antagonist is administered in an induction phase and a consolidation phase. In certain embodiments, during the induction phase, the FcRn antagonist is administered once weekly or more frequently, e.g., twice a week or every other day. In certain embodiments, during the induction phase, the FcRn antagonist is administered less frequently than once weekly, e.g., once every other week. In certain embodiments, (i) during the induction phase the FcRn antagonist is administered once weekly and corticosteroid (e.g., 0.5 mg prednisone/kg/day or equivalent OCS) is administered until disease control, and (ii) during the consolidation phase the FcRn antagonist dose is decreased and/or the FcRn antagonist dosing interval is lengthened, e.g., to once biweekly, and/or the corticosteroid dose is decreased and/or the corticosteroid dosing interval is lengthened, to an end-of-consolidation dose or dosing interval effective to prevent new lesions from appearing.

In certain embodiments, (i) during the induction phase the FcRn antagonist is administered once weekly and corticosteroid (e.g., 0.5 mg prednisone/kg/day or equivalent OCS) is administered until disease control, and (ii) during the consolidation phase the FcRn antagonist dose is decreased and/or the FcRn antagonist dosing interval is lengthened, e.g., to once biweekly, to an end-of-consolidation dose or dosing interval effective to prevent new lesions from appearing.

In certain embodiments, (i) during the induction phase the FcRn antagonist is administered once weekly and corticosteroid (e.g., 0.5 mg prednisone/kg/day or equivalent OCS) is administered until disease control, and (ii) during the consolidation phase the corticosteroid dose is decreased and/or the corticosteroid dosing interval is lengthened, to an end-of-consolidation dose or dosing interval effective to prevent new lesions from appearing.

In certain embodiments, (i) during the induction phase the FcRn antagonist is administered once weekly and corticosteroid (e.g., 0.5 mg prednisone/kg/day or equivalent OCS) is administered until disease control, and (ii) during the consolidation phase the FcRn antagonist dose is decreased and/or the FcRn antagonist dosing interval is lengthened, e.g., to once biweekly, and the corticosteroid dose is decreased and/or the corticosteroid dosing interval is lengthened, to an end-of-consolidation dose or dosing interval effective to prevent new lesions from appearing.

In some embodiments, during the induction phase the FcRn antagonist is administered subcutaneously at a fixed dose of about 750 mg to about 3000 mg. In some embodiments, during the induction phase the FcRn antagonist is administered subcutaneously at a fixed dose of about 750 mg to about 2000 mg. In some embodiments, during the induction phase the FcRn antagonist is administered subcutaneously at a fixed dose of about 750 mg. In some embodiments, during the induction phase the FcRn antagonist is administered subcutaneously at a fixed dose of about 1000 mg. In some embodiments, during the induction phase the FcRn antagonist is administered subcutaneously at a fixed dose of about 1250 mg. In some embodiments, during the induction phase the FcRn antagonist is administered subcutaneously at a fixed dose of about 1500 mg. In some embodiments, during the induction phase the FcRn antagonist is administered subcutaneously at a fixed dose of about 1750 mg. In some embodiments, during the induction phase the FcRn antagonist is administered subcutaneously at a fixed dose of about 2000 mg.

In some embodiments, during the induction phase the FcRn antagonist is administered subcutaneously at a fixed dose of 750 mg to 3000 mg. In some embodiments, during the induction phase the FcRn antagonist is administered subcutaneously at a fixed dose of 750 mg to 2000 mg. In some embodiments, during the induction phase the FcRn antagonist is administered subcutaneously at a fixed dose of 750 mg. In some embodiments, during the induction phase the FcRn antagonist is administered subcutaneously at a fixed dose of 1000 mg. In some embodiments, during the induction phase the FcRn antagonist is administered subcutaneously at a fixed dose of 1250 mg. In some embodiments, during the induction phase the FcRn antagonist is administered subcutaneously at a fixed dose of 1500 mg. In some embodiments, during the induction phase the FcRn antagonist is administered subcutaneously at a fixed dose of 1750 mg. In some embodiments, during the induction phase the FcRn antagonist is administered subcutaneously at a fixed dose of 2000 mg.

In some embodiments, during the induction phase the FcRn antagonist is administered subcutaneously at a dose of about 10 mg/kg to about 25 mg/kg. In some embodiments, during the induction phase the FcRn antagonist is administered subcutaneously at a dose of about 10 mg/kg. In some embodiments, during the induction phase the FcRn antagonist is administered subcutaneously at a dose of about 15 mg/kg. In some embodiments, during the induction phase the FcRn antagonist is administered subcutaneously at a dose of about 20 mg/kg.

In some embodiments, during the induction phase the FcRn antagonist is administered subcutaneously at a dose of about 25 mg/kg.

In some embodiments, during the induction phase the FcRn antagonist is administered subcutaneously at a dose of 10 mg/kg to 25 mg/kg. In some embodiments, during the induction phase the FcRn antagonist is administered subcutaneously at a dose of 10 mg/kg. In some embodiments, during the induction phase the FcRn antagonist is administered subcutaneously at a dose of 15 mg/kg. In some embodiments, during the induction phase the FcRn antagonist is administered subcutaneously at a dose of 20 mg/kg. In some embodiments, during the induction phase the FcRn antagonist is administered subcutaneously at a dose of 25 mg/kg.

In some embodiments, during the induction phase the FcRn antagonist is administered intravenously at a dose of about 10 mg/kg to about 25 mg/kg. In some embodiments, during the induction phase the FcRn antagonist is administered intravenously at a dose of about 10 mg/kg. In some embodiments, during the induction phase the FcRn antagonist is administered intravenously at a dose of about 15 mg/kg. In some embodiments, during the induction phase the FcRn antagonist is administered intravenously at a dose of about 20 mg/kg. In some embodiments, during the induction phase the FcRn antagonist is administered intravenously at a dose of about 25 mg/kg.

In some embodiments, during the induction phase the FcRn antagonist is administered intravenously at a dose of 10 mg/kg to 25 mg/kg. In some embodiments, during the induction phase the FcRn antagonist is administered intravenously at a dose of 10 mg/kg. In some embodiments, during the induction phase the FcRn antagonist is administered intravenously at a dose of 15 mg/kg. In some embodiments, during the induction phase the FcRn antagonist is administered intravenously at a dose of 20 mg/kg. In some embodiments, during the induction phase the FcRn antagonist is administered intravenously at a dose of 25 mg/kg.

In some embodiments, during the consolidation phase the FcRn antagonist dosing interval is once weekly or less frequently. For example, in certain embodiments, during the consolidation phase the FcRn antagonist dosing interval is once every 7, 8, 9, 10, 11, 12, 13, 14, 15, 16, 17, 18, 19, 20, or 21 days; or once every 2, 3, 4, 5, or 6 weeks. In certain embodiments, during the consolidation phase the FcRn antagonist dosing interval is once every 7 to 8, 7 to 9, 7 to 10, 7 to 11, 7 to 12, 7 to 13, 7 to 14, 7 to 15, 7 to 16, 7 to 17, 7 to 18, 7 to 19, 7 to 20, or 7 to 21 days. In certain embodiments, during the consolidation phase the FcRn antagonist dosing interval is once every 8 to 9, 8 to 10, 8 to 11, 8 to 12, 8 to 13, 8 to 14, 8 to 15, 8 to 16, 8 to 17, 8 to 18, 8 to 19, 8 to 20, or 8 to 21 days. In certain embodiments, during the consolidation phase the FcRn antagonist dosing interval is once every 9 to 10, 9 to 11, 9 to 12, 9 to 13, 9 to 14, 9 to 15, 9 to 16, 9 to 17, 9 to 18, 9 to 19, 9 to 20, or 9 to 21 days. In certain embodiments, during the consolidation phase the FcRn antagonist dosing interval is once every 10 to 11, 10 to 12, 10 to 13, 10 to 14, 10 to 15, 10 to 16, 10 to 17, 10 to 18, 10 to 19, 10 to 20, or 10 to 21 days. In certain embodiments, during the consolidation phase the FcRn antagonist dosing interval is once every 11 to 12, 11 to 13, 11 to 14, 11 to 15, 11 to 16, 11 to 17, 11 to 18, 11 to 19, 11 to 20, or 11 to 21 days. In certain embodiments, during the consolidation phase the FcRn antagonist dosing interval is once every 12 to 13, 12 to 14, 12 to 15, 12 to 16, 12 to 17, 12 to 18, 12 to 19, 12 to 20, or 12 to 21 days. In certain embodiments, during the consolidation phase the FcRn antagonist dosing interval is once every 13 to 14, 13 to 15, 13 to 16, 13 to 17, 13 to 18, 13 to 19, 13 to 20, or 13 to 21 days. In certain embodiments, during the consolidation phase the FcRn antagonist dosing interval is once every 14 to 15, 14 to 16, 14 to 17, 14 to 18, 14 to 19, 14 to 20, or 14 to 21 days. In certain embodiments, during the consolidation phase the FcRn antagonist dosing interval is once every 15 to 16, 15 to 17, 15 to 18, 15 to 19, 15 to 20, or 15 to 21 days. In certain embodiments, during the consolidation phase the FcRn antagonist dosing interval is once every 16 to 17, 16 to 18, 16 to 19, 16 to 20, or 16 to 21 days. In certain embodiments, during the consolidation phase the FcRn antagonist dosing interval is once every 17 to 18, 17 to 19, 17 to 20, or 17 to 21 days. In certain embodiments, during the consolidation phase the FcRn antagonist dosing interval is once every 18 to 19, 18 to 20, or 18 to 21 days. In certain embodiments, during the consolidation phase the FcRn antagonist dosing interval is once every 19 to 20, or 19 to 21 days.

In some embodiments, during the consolidation phase the FcRn antagonist dosing interval is once weekly or every 2 weeks.

In some embodiments, the method further comprises a maintenance phase, wherein during the maintenance phase the end-of-consolidation dose or dosing interval for the FcRn antagonist and/or the corticosteroid is continued until complete clearance of lesions.

In some embodiments, during the maintenance phase the FcRn antagonist dosing interval is once weekly or less frequently. For example, in certain embodiments, during the maintenance phase the FcRn antagonist dosing interval is once every 7, 8, 9, 10, 11, 12, 13, 14, 15, 16, 17, 18, 19, 20, or 21 days; or once every 2, 3, 4, 5, or 6 weeks. In certain embodiments, during the maintenance phase the FcRn antagonist dosing interval is once every 7 to 8, 7 to 9, 7 to 10, 7 to 11, 7 to 12, 7 to 13, 7 to 14, 7 to 15, 7 to 16, 7 to 17, 7 to 18, 7 to 19, 7 to 20, or 7 to 21 days. In certain embodiments, during the maintenance phase the FcRn antagonist dosing interval is once every 8 to 9, 8 to 10, 8 to 11, 8 to 12, 8 to 13, 8 to 14, 8 to 15, 8 to 16, 8 to 17, 8 to 18, 8 to 19, 8 to 20, or 8 to 21 days. In certain embodiments, during the maintenance phase the FcRn antagonist dosing interval is once every 9 to 10, 9 to 11, 9 to 12, 9 to 13, 9 to 14, 9 to 15, 9 to 16, 9 to 17, 9 to 18, 9 to 19, 9 to 20, or 9 to 21 days. In certain embodiments, during the maintenance phase the FcRn antagonist dosing interval is once every 10 to 11, 10 to 12, 10 to 13, 10 to 14, 10 to 15, 10 to 16, 10 to 17, 10 to 18, 10 to 19, 10 to 20, or 10 to 21 days. In certain embodiments, during the maintenance phase the FcRn antagonist dosing interval is once every 11 to 12, 11 to 13, 11 to 14, 11 to 15, 11 to 16, 11 to 17, 11 to 18, 11 to 19, 11 to 20, or 11 to 21 days. In certain embodiments, during the maintenance phase the FcRn antagonist dosing interval is once every 12 to 13, 12 to 14, 12 to 15, 12 to 16, 12 to 17, 12 to 18, 12 to 19, 12 to 20, or 12 to 21 days. In certain embodiments, during the maintenance phase the FcRn antagonist dosing interval is once every 13 to 14, 13 to 15, 13 to 16, 13 to 17, 13 to 18, 13 to 19, 13 to 20, or 13 to 21 days. In certain embodiments, during the maintenance phase the FcRn antagonist dosing interval is once every 14 to 15, 14 to 16, 14 to 17, 14 to 18, 14 to 19, 14 to 20, or 14 to 21 days. In certain embodiments, during the maintenance phase the FcRn antagonist dosing interval is once every 15 to 16, 15 to 17, 15 to 18, 15 to 19, 15 to 20, or 15 to 21 days. In certain embodiments, during the maintenance phase the FcRn antagonist dosing interval is once every 16 to 17, 16 to 18, 16 to 19, 16 to 20, or 16 to 21 days. In certain embodiments, during the maintenance phase the FcRn antagonist dosing interval is once every 17 to 18, 17 to 19, 17 to 20, or 17 to 21 days. In certain embodiments, during the maintenance phase the FcRn antagonist dosing interval is once every 18 to 19, 18 to 20, or 18 to 21 days. In certain embodiments, during the maintenance phase the FcRn antagonist dosing interval is once every 19 to 20, or 19 to 21 days.

In some embodiments, during the maintenance phase the FcRn antagonist dosing interval is once weekly. In some embodiments, during the maintenance phase the FcRn antagonist dosing interval is once biweekly.

In some embodiments, during the maintenance phase the corticosteroid is administered at a dose of ≤0.1 mg/kg/day prednisone or equivalent OCS.

In some embodiments, during the maintenance phase the FcRn antagonist is administered once weekly subcutaneously at a dose of 1000 mg and the corticosteroid is administered at a dose of ≤0.1 mg/kg/day prednisone or equivalent OCS.

In an embodiment, the subject has a serum level of a pathogenic IgG autoantibody that is associated with BP. In an embodiment, the pathogenic IgG autoantibody is an anti-BP180 antibody and/or an anti-BP230 antibody. In some embodiments, the anti-BP180 antibody is an anti-NC16A antibody. In an embodiment, the level of a pathogenic IgG autoantibody is measured by ELISA or addressable laser bead immunoassay (ALBIA). In an embodiment, the serum level of a pathogenic IgG autoantibody is compared to baseline levels in the subject.

In an embodiment, the BP is newly diagnosed BP, relapsing BP, or refractory BP. In some embodiments, the subject is diagnosed with moderate BP or severe BP. In some embodiments, the subject is diagnosed with moderate-to-severe BP. In some embodiments, the subject is diagnosed according to the IGA-BP scale. In some embodiments, the subject has an IGA-BP score of 3 or 4.

In some embodiments, treatment of BP is characterized by the reduction of autoantibodies. In some embodiments, the autoantibodies are anti-BP180 autoantibodies. In some embodiments, the prevalence of anti-BP180 autoantibodies is reduced by at least 10%, at least 25%, at least 50%, at least 75%, at least 80%, at least 90%, at least 95%, at least 96%, at least 97%, at least 98%, or at least 99% after administering one or more of the treatments described herein. In some embodiments, anti-BP180 autoantibodies are undetectable in the subject after administering one or more of the treatments described herein. In some embodiments, the treatment is administration of an effective amount of an FcRn antagonist. In some embodiments, the treatment is administration of an effective amount of efgartigimod. In some embodiments, the anti-BP180 autoantibody is an anti-NC16A autoantibody.

In some embodiments, the autoantibodies are anti-BP230 autoantibodies. In some embodiments, the prevalence of anti-BP230 autoantibodies is reduced by at least 10%, at least 25%, at least 50%, at least 75%, at least 80%, at least 90%, at least 95%, at least 96%, at least 97%, at least 98%, or at least 99% after administering one or more of the treatments described herein. In some embodiments, anti-BP230 autoantibodies are undetectable in the subject after administering one or more of the treatments described herein. In some embodiments, the treatment is administration of an effective amount of an FcRn antagonist. In some embodiments, the treatment is administration of an effective amount of efgartigimod.

In some embodiments, treatment of BP is characterized by change in BP disease status. Various terms are used to describe BP disease status and are defined elsewhere herein. In some embodiments, change in BP disease status is characterized as one or more of the following: control of disease activity (CDA), partial remission (PR), complete remission (CR), relapse, and/or treatment failure. In some embodiments, CDA, PR, or CR is achieved after administering one or more of the treatments described herein. In some embodiments, CDA, PR, or CR is observed within ≤36 weeks, ≤26 weeks, ≤12 weeks, <6 weeks, or ≤3 weeks after administering one or more of the treatments described herein. In some embodiments, CDA, PR, or CR is maintained in the absence of corticosteroid therapy. In some embodiments, CDA, PR, or CR is maintained for at least 2 weeks, at least 4 weeks, at least 8 weeks, at least 12 weeks, at least 16 weeks, or at least 26 weeks off corticosteroid therapy.

In some embodiments, treatment prevents BP relapse. In some embodiments, treatment prevents BP relapse for at least 2 weeks, at least 4 weeks, at least 8 weeks, at least 12 weeks, at least 16 weeks, at least 26 weeks, at least 36 weeks, at least 40 weeks, at least 44 weeks, at least 48 weeks, or at least 52 weeks. In some embodiments, the treatment is administration of an effective amount of an FcRn antagonist. In some embodiments, the treatment is administration of an effective amount of efgartigimod.

In some embodiments, treatment prevents BP relapse after achieving remission from BP, for at least 2 weeks, at least 4 weeks, at least 8 weeks, at least 12 weeks, at least 16 weeks, at least 26 weeks, at least 36 weeks, at least 40 weeks, at least 44 weeks, at least 48 weeks, or at least 52 weeks after the initiation of the treatment with the FcRn antagonist. In some embodiments, the initiation of the treatment with the FcRn antagonist is the first administration of an effective amount of the FcRn antagonist. In some embodiments, the initiation of the treatment with the FcRn antagonist is the first administration of an effective amount of efgartigimod.

In some embodiments, the treatment prevents BP relapse after achieving remission from BP for at least 4 weeks after achieving remission from BP. In some embodiments, the treatment prevents BP relapse after achieving remission from BP for at least 4, 6, 8, 10, 12, 14, 16, 18, 20, 22, 24, 26, 28, 30, 32, 34, 36, 38, 40, 42, 44, 46, 48, 50, or 52 weeks after achieving remission from BP.

In some embodiments, CR is achieved at a corticosteroid dose of ≤about 5 mg prednisone/kg/day or equivalent OCS. In some embodiments, CR is achieved at a corticosteroid dose of ≤about 3 mg prednisone/kg/day or equivalent OCS. In some embodiments, CR is achieved at a corticosteroid dose of ≤about 2 mg prednisone/kg/day or equivalent OCS. In some embodiments, CR is achieved at a corticosteroid dose of ≤about 1 mg prednisone/kg/day or equivalent OCS. In some embodiments, CR is achieved at a corticosteroid dose of ≤about 0.5 mg prednisone/kg/day or equivalent OCS. In some embodiments, CR is achieved at a corticosteroid dose of ≤about 0.4 mg prednisone/kg/day or equivalent OCS. In some embodiments, CR is achieved at a corticosteroid dose of ≤about 0.3 mg prednisone/kg/day or equivalent OCS. In some embodiments, CR is achieved at a corticosteroid dose of ≤about 0.2 mg prednisone/kg/day or equivalent OCS. In some embodiments, CR is achieved at a corticosteroid dose of ≤about 0.1 mg prednisone/kg/day or equivalent OCS.

In some embodiments, CR is achieved at a corticosteroid dose of ≤5 mg prednisone/kg/day or equivalent OCS. In some embodiments, CR is achieved at a corticosteroid dose of ≤3 mg prednisone/kg/day or equivalent OCS. In some embodiments, CR is achieved at a corticosteroid dose of ≤2 mg prednisone/kg/day or equivalent OCS. In some embodiments, CR is achieved at a corticosteroid dose of ≤1 mg prednisone/kg/day or equivalent OCS. In some embodiments, CR is achieved at a corticosteroid dose of ≤0.5 mg prednisone/kg/day or equivalent OCS. In some embodiments, CR is achieved at a corticosteroid dose of ≤0.4 mg prednisone/kg/day or equivalent OCS. In some embodiments, CR is achieved at a corticosteroid dose of ≤0.3 mg prednisone/kg/day or equivalent OCS. In some embodiments, CR is achieved at a corticosteroid dose of ≤0.2 mg prednisone/kg/day or equivalent OCS. In some embodiments, CR is achieved at a corticosteroid dose of ≤0.1 mg prednisone/kg/day or equivalent OCS.

In some embodiments, CR is achieved at a corticosteroid dose of ≤about 20 mg prednisone/day or equivalent OCS. In some embodiments, CR is achieved at a corticosteroid dose of ≤about 15 mg prednisone/day or equivalent OCS. In some embodiments, CR is achieved at a corticosteroid dose of ≤about 10 mg prednisone/day or equivalent OCS. In some embodiments, CR is achieved at a corticosteroid dose of ≤about 5 mg prednisone/day or equivalent OCS. In some embodiments, CR is achieved at a corticosteroid dose of ≤about 3 mg prednisone/day or equivalent OCS. In some embodiments, CR is achieved at a corticosteroid dose of ≤about 2 mg prednisone/day or equivalent OCS.

In some embodiments, CR is achieved at a corticosteroid dose of ≤10 mg prednisone/day or equivalent OCS. In some embodiments, CR is achieved at a corticosteroid dose of ≤5 mg prednisone/day or equivalent OCS. In some embodiments, CR is achieved at a corticosteroid dose of ≤3 mg prednisone/day or equivalent OCS. In some embodiments, CR is achieved at a corticosteroid dose of ≤2 mg prednisone/day or equivalent OCS.

In some embodiments, CR is achieved without corticosteroid.

In some embodiments, CR is maintained with a corticosteroid dose of ≤about 20 mg prednisone/day or equivalent OCS. In some embodiments, CR is maintained with a corticosteroid dose of ≤about 15 mg prednisone/day or equivalent OCS. In some embodiments, CR is maintained with a corticosteroid dose of ≤about 10 mg prednisone/day or equivalent OCS. In some embodiments, CR is maintained with a corticosteroid dose of ≤about 5 mg prednisone/day or equivalent OCS. In some embodiments, CR is maintained with a corticosteroid dose of ≤about 2 mg prednisone/day or equivalent OCS. In some embodiments, CR remission is maintained with a corticosteroid dose of ≤about 1 mg prednisone/kg/day or equivalent OCS. In some embodiments, CR is maintained with a corticosteroid dose of ≤about 0.5 mg prednisone/kg/day or equivalent OCS. In some embodiments, CR is maintained with a corticosteroid dose of ≤about 0.4 mg prednisone/kg/day or equivalent OCS. In some embodiments, CR is maintained with a corticosteroid dose of ≤about 0.3 mg prednisone/kg/day or equivalent OCS. In some embodiments, CR is maintained with a corticosteroid dose of ≤about 0.2 mg prednisone/kg/day or equivalent OCS. In some embodiments, CR is maintained with a corticosteroid dose of ≤about 0.1 mg prednisone/kg/day or equivalent OCS.

In some embodiments, CR is maintained with a corticosteroid dose of ≤20 mg prednisone/day or equivalent OCS. In some embodiments, CR is maintained with a corticosteroid dose of ≤15 mg prednisone/day or equivalent OCS. In some embodiments, CR is maintained with a corticosteroid dose of ≤10 mg prednisone/day or equivalent OCS. In some embodiments, CR is maintained with a corticosteroid dose of ≤5 mg prednisone/day or equivalent OCS. In some embodiments, CR is maintained with a corticosteroid dose of ≤2 mg prednisone/day or equivalent OCS. In some embodiments, CR is maintained with a corticosteroid dose of ≤1 mg prednisone/kg/day or equivalent OCS. In some embodiments, CR is maintained with a corticosteroid dose of ≤0.5 mg prednisone/kg/day or equivalent OCS. In some embodiments, CR is maintained with a corticosteroid dose of ≤0.4 mg prednisone/kg/day or equivalent OCS. In some embodiments, CR is maintained with a corticosteroid dose of ≤0.3 mg prednisone/kg/day or equivalent OCS. In some embodiments, CR is maintained with a corticosteroid dose of ≤0.2 mg prednisone/kg/day or equivalent OCS. In some embodiments, CR is maintained with a corticosteroid dose of ≤0.1 mg prednisone/kg/day or equivalent OCS.

In some embodiments, CR is maintained in the absence of corticosteroid.

In some embodiments, treatment of BP is characterized by decrease in Investigator Global Assessment of Bullous Pemphigoid (IGA-BP). In some embodiments, IGA-BP is decreased by 1 point, by 2 points, by 3 points, or by 4 points after administering one or more of the treatments described herein. In some embodiments, IGA-BP is decreased from 3 or 4 to 0 or 1 after administering one or more of the treatments described herein. In some embodiments, IGA-BP is decreased from 3 or 4 to 0 after administering one or more of the treatments described herein. In some embodiments, an IGA-BP score of 0 or 1 is maintained on minimal OCS therapy. In some embodiments, an IGA-BP score of 0 or 1 is maintained for at least 2 weeks, at least 4 weeks, at least 8 weeks, at least 12 weeks, at least 16 weeks, or at least 26 weeks on minimal OCS therapy. In some embodiments, an IGA-BP score of 0 or 1 is maintained in the absence of corticosteroid therapy. In some embodiments, an IGA-BP score of 0 or 1 is maintained for at least 2 weeks, at least 4 weeks, at least 8 weeks, at least 12 weeks, at least 16 weeks, or at least 26 weeks off corticosteroid therapy. In some embodiments, the treatment is administration of an effective amount of an FcRn antagonist. In some embodiments, the treatment is administration of an effective amount of efgartigimod.

In some embodiments, treatment of BP is characterized by decrease in bullous pemphigoid disease area index (BPDAI). In some embodiments, the BPDAI is decreased by at least 5%, at least 10%, at least 15%, at least 20%, at least 25%, at least 30%, at least 35%, at least 40%, at least 45%, at least 50%, at least 55%, at least 60%, at least 70%, at least 80%, at least 90%, at least 95%, or 100% after administering one or more of the treatments described herein. In some embodiments, the BPDAI is decreased to ≤36, ≤24, ≤18, ≤12, <8, <4, or 0. In some embodiments, the treatment is administration of an effective amount of an FcRn antagonist. In some embodiments, the treatment is administration of an effective amount of efgartigimod.

In some embodiments, treatment of BP is characterized by decrease in Itch Numerical Rating Scale (Itch NRS) score. In some embodiments, the Itch NRS score is decreased by at least 1 point, at least 2 points, at least 3 points, at least 4 points, at least 5 points, at least 6 points, at least 7 points, at least 8 points, at least 9 points, or at least 10 points after administering one or more of the treatments described herein. In some embodiments, the Itch NRS score is <7, <6, <5, <4, <3, <2, or <1 after administering one or more of the treatments described herein. In some embodiments, the Itch NRS score is 0, 1, or 2 after administering one or more of the treatments described herein. In some embodiments, the treatment is administration of an effective amount of an FcRn antagonist. In some embodiments, the treatment is administration of an effective amount of efgartigimod.

In some embodiments, treatment of BP is characterized by improvement in quality of life. In some embodiments, quality of life is assessed by one or more of the following: EuroQOL 5-Dimension 5-Level (EQ-5D-5L), Dermatology Life Quality Index (DLQI), and/or Autoimmune Bullous Disease Quality of Life (ABQOL). In some embodiments, quality of life is improved by at least 5%, at least 10%, at least 15%, at least 20%, at least 25%, at least 30%, at least 35%, at least 40%, at least 45%, at least 50%, at least 55%, at least 60%, at least 70%, at least 80%, at least 90%, or at least 95% after administering one or more of the treatments described herein. In some embodiments, the treatment is administration of an effective amount of an FcRn antagonist. In some embodiments, the treatment is administration of an effective amount of efgartigimod.

In some embodiments, treatment of BP is characterized by improvement in glucocorticoid toxicity index (GTI). In some embodiments, GTI score is decreased by at least 5%, at least 10%, at least 15%, at least 20%, at least 25%, at least 30%, at least 35%, at least 40%, at least 45%, at least 50%, at least 55%, at least 60%, at least 70%, at least 80%, at least 90%, at least 95%, or 100% after administering one or more of the treatments described herein. In some embodiments, the treatment is administration of an effective amount of an FcRn antagonist. In some embodiments, the treatment is administration of an effective amount of efgartigimod.

In an aspect, a method of treating BP in a subject in need thereof is provided, the method comprising administering to the subject an effective amount of a human FcRn antagonist and a corticosteroid, wherein a cumulative oral dose of the corticosteroid administered to the subject does not exceed 100 mg/kg. In some embodiments, the cumulative oral dose of the corticosteroid does not exceed 75 mg/kg. In some embodiments, the cumulative oral dose of the corticosteroid does not exceed 50 mg/kg. In some embodiments, the cumulative oral dose of the corticosteroid does not exceed 25 mg/kg. In some embodiments, the corticosteroid is prednisone. In some embodiments, the cumulative oral dose of the corticosteroid is a cumulative oral prednisone equivalent dose.

In an aspect, a method of treating BP in a subject in need thereof is provided, the method comprising administering to the subject an effective amount of a human FcRn antagonist and a corticosteroid, wherein an oral prednisone equivalent dose of the corticosteroid administered to the subject does not exceed 1 mg/kg/day. In some embodiments, the oral prednisone equivalent dose of the corticosteroid administered to the subject does not exceed 0.75 mg/kg/day. In some embodiments, the oral prednisone equivalent dose of the corticosteroid administered to the subject does not exceed 0.5 mg/kg/day. In some embodiments, the oral prednisone equivalent dose of the corticosteroid administered to the subject does not exceed 0.3 mg/kg/day. In some embodiments, the oral prednisone equivalent dose of the corticosteroid administered to the subject does not exceed 0.25 mg/kg/day. In some embodiments, the oral prednisone equivalent dose of the corticosteroid administered to the subject does not exceed 0.2 mg/kg/day. In some embodiments, the oral prednisone equivalent dose of the corticosteroid administered to the subject does not exceed 0.15 mg/kg/day. In some embodiments, the oral prednisone equivalent dose of the corticosteroid administered to the subject does not exceed 0.1 mg/kg/day. In some embodiments, the corticosteroid is prednisone.

In an aspect, a method of treating BP in a subject in need thereof is provided, the method comprising administering to the subject an effective amount of a human FcRn antagonist and a corticosteroid, wherein an oral prednisone equivalent dose of the corticosteroid administered to the subject does not exceed 12.5 mg/day. In some embodiments, the oral prednisone equivalent dose of the corticosteroid administered to the subject does not exceed 10 mg/day. In some embodiments, the corticosteroid is prednisone.

In an aspect, provided herein is a method for monitoring treatment of BP in a subject following treatment with an FcRn antagonist, the method comprising: a) measuring an autoantibody in a blood sample taken from the subject; and b) comparing the autoantibody value to a reference value associated with BP in the subject, wherein the subject is in remission from BP if the autoantibody in the sample is lower than or equal to the reference value.

In an aspect, provided herein is a method for monitoring efficacy of treatment of BP in a subject following treatment with a first FcRn antagonist, the method comprising: a) measuring in vitro a serum level of a BP180 antibody and/or BP230 antibody in a blood sample taken from the subject; and b) comparing the serum level of the BP180 antibody and/or BP230 antibody to a reference value associated with BP in the subject, wherein the treatment is not effective if the serum level of the BP180 antibody and/or BP230 antibody in the sample is greater than or equal to the reference value, and wherein the treatment is effective if the serum level of the BP180 antibody and/or BP230 antibody in the sample is less than the reference value. In some embodiments, the BP180 antibody is an NC16A antibody.

In an aspect, provided herein is a method of treating BP in a subject that has received a first FcRn antagonist and is receiving a corticosteroid dosing regimen, the method comprising: a) administering to the subject an effective amount of a second FcRn antagonist; b) measuring in vitro a serum level of a BP180 antibody and/or BP230 antibody in a blood sample taken from the subject; and c) comparing the serum level of the BP180 antibody and/or BP230 antibody to a reference value associated with BP in the subject, wherein the corticosteroid dosing regimen is maintained if the serum level of the BP180 antibody and/or BP230 antibody in the sample is greater than or equal to the reference value, or wherein corticosteroid dosing regimen is tapered if the serum level of the BP180 antibody and/or BP230 antibody is less than the reference value. In some embodiments, the BP180 antibody is an NC16A antibody.

In an aspect, provided herein is a second FcRn antagonist for use in a method of treating BP in a subject that has received a first FcRn antagonist and is receiving a corticosteroid dosing regimen, wherein: a) an effective amount of the second FcRn antagonist is administered to the subject; b) a serum level of a BP180 antibody and/or BP230 antibody in a blood sample taken from the subject is measured in vitro; and c) the serum level of the BP180 antibody and/or BP230 antibody is compared to a reference value associated with BP in the subject, wherein the corticosteroid dosing regimen is maintained if the serum level of the BP180 antibody and/or BP230 antibody in the sample is greater than or equal to the reference value, and wherein corticosteroid dosing regimen is tapered if the serum level of the BP180 antibody and/or BP230 antibody is less than the reference value. In some embodiments, the BP180 antibody is an NC16A antibody.

In some embodiments, the corticosteroid dose regimen is tapered to a lower dose amount or a lower dosing frequency. In an embodiment, the method further comprises administering to the subject an effective amount of the second FcRn antagonist if the serum level of the BP180 antibody and/or BP230 antibody in the sample is greater than or equal to the reference value. In an embodiment, the method further comprises administering to the subject an effective amount of a second FcRn antagonist if the serum level of the BP180 antibody and/or BP230 antibody in the sample is greater than or equal to the reference value. In some embodiments, the BP180 antibody is an NC16A antibody.

In an aspect, provided herein is a method for treating BP in a subject comprising: (a) administering to the subject one or more initial doses of an effective amount of a first FcRn antagonist, (b) administering to the subject one or more further doses of an effective amount of a second FcRn antagonist if the serum level of a BP180 antibody and/or BP230 antibody in the subject after step (a) is greater than or equal to a reference value associated with BP in the subject, or discontinuing treatment with the first FcRn antagonist if the serum level of the BP180 antibody and/or BP230 antibody in the subject after step (a) is less than a reference value associated with active disease in the subject. In some embodiments, the BP180 antibody is an NC16A antibody.

In an aspect, provided herein is an FcRn antagonist for use in a method of treating BP in a subject, wherein (a) one or more initial doses of an effective amount of a first FcRn antagonist is administered to the subject, and (b) one or more further doses of an effective amount of a second FcRn antagonist is administered to the subject if the serum level of a BP180 antibody and/or BP230 antibody in the subject after step (a) is greater than or equal to a reference value associated with BP in the subject or the first FcRn antagonist is discontinued if the serum level of the BP180 antibody and/or BP230 antibody in the subject after step (a) is less than a reference value associated with BP in the subject. In some embodiments, the BP180 antibody is an NC16A antibody.

In an embodiment, the effective amount of the first FcRn antagonist is a dose of about 10 mg/kg to about 30 mg/kg, administered intravenously.

In an embodiment, the effective amount of the first FcRn antagonist is a dose of about 750 mg to about 3000 mg, administered subcutaneously.

In an aspect, provided herein is a method for determining if a subject that has previously been treated for BP using a first FcRn antagonist requires further treatment with a second FcRn antagonist, the method comprising: a) measuring in vitro a serum level of a BP180 antibody and/or BP230 antibody in a blood sample taken from the subject; and b) comparing the serum level of the BP180 antibody and/or BP230 antibody to a reference value associated with BP in the subject, wherein if the serum level of the BP180 antibody and/or BP230 antibody in the sample is greater than or equal to the reference value, then the subject is need of further treatment with the second FcRn antagonist. In some embodiments, the BP180 antibody is an NC16A antibody.

In an aspect, provided herein is a method for treating BP in a subject comprising: administering to the subject an effective amount of a second FcRn antagonist, wherein the BP has relapsed in the subject following prior therapy with a first FcRn antagonist and wherein the subject has a serum level of a BP180 antibody and/or BP230 antibody that is greater than or equal to a reference value associated with BP in the subject. In some embodiments, the BP180 antibody is an NC16A antibody.

In an aspect, provided herein is a second FcRn antagonist for use in a method of treating BP in a subject, wherein the BP has relapsed in the subject following prior therapy with a first FcRn antagonist and wherein the subject has a serum level of a BP180 antibody and/or BP230 antibody that is greater than or equal to a reference value associated with BP in the subject. In some embodiments, the BP180 antibody is an NC16A antibody.

In an aspect, provided herein is a method for treating BP in a subject comprising administering to the subject an effective amount of a second FcRn antagonist, wherein the BP has relapsed in the subject following prior therapy with a first FcRn antagonist, and wherein the effective amount of the FcRn antagonist is determined based on the serum level of a BP180 antibody and/or BP230 antibody in a blood sample taken from the subject. In some embodiments, the BP180 antibody is an NC16A antibody.

In an aspect, provided herein is a second FcRn antagonist for use in a method of treating BP in a subject, wherein the BP has relapsed in the subject following prior therapy with a first FcRn antagonist, the method comprising administering to the subject an effective amount of a second FcRn antagonist, wherein the effective amount is determined based on the serum level of a BP180 antibody and/or BP230 antibody in a blood sample taken from the subject. In some embodiments, the BP180 antibody is an NC16A antibody.

In an aspect, provided herein is a method for monitoring treatment efficacy in a subject following treatment with a first FcRn antagonist, wherein the subject has BP, the method comprising: a) measuring in vitro a serum level of a BP180 antibody and/or BP230 antibody in a blood sample taken from the subject; and b) comparing the serum level of the BP180 antibody and/or BP230 antibody to a reference value associated with BP in the subject, wherein the treatment is not effective if the serum level of the BP180 antibody and/or BP230 antibody in the sample is greater than or equal to the reference value, or wherein the treatment is effective if the serum level of the BP180 antibody and/or BP230 antibody is less than the reference value. In some embodiments, the BP180 antibody is an NC16A antibody.

In an aspect, provided herein is a method for monitoring remission of BP in a subject following treatment with a first FcRn antagonist, the method comprising: a) measuring in vitro a serum level of a BP180 antibody and/or BP230 antibody in a blood sample taken from the subject; and b) comparing the serum level of the BP180 antibody and/or BP230 antibody to a reference value associated with BP in the subject, wherein the subject is in remission from BP if the serum level of the BP180 antibody and/or BP230 antibody in the sample is lower than or equal to the reference value. In some embodiments, the BP180 antibody is an NC16A antibody.

BP180 antibodies and BP230 antibodies are typically not present in serum of healthy individuals (i.e., an individual not suffering from BP or other autoantibody-mediated disease). Thus, in an embodiment, the reference value is an undetectable serum level of a BP180 antibody and/or BP230 antibody. In some embodiments, the BP180 antibody is an NC16A antibody.

In some embodiments, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the maximum serum level of the BP180 antibody or the BP230 antibody measured in the subject prior to receiving any treatment for BP. In some embodiments, the BP180 antibody is an NC16A antibody.

In some embodiments, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the maximum serum level of the BP180 antibody or the BP230 antibody measured in the subject prior to receiving any treatment for BP. In some embodiments, the BP180 antibody is an NC16A antibody.

In some embodiments, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mean serum level of the BP180 antibody or the BP230 antibody measured in the subject prior to receiving any treatment for BP. In some embodiments, the BP180 antibody is an NC16A antibody.

In an embodiment, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% of the mean serum level of the BP180 antibody or the BP230 antibody measured in the subject prior to receiving any treatment for BP. In some embodiments, the BP180 antibody is an NC16A antibody.

In an embodiment, the reference value is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% higher than the lowest serum level of the BP180 antibody or the BP230 antibody measured in the subject following treatment with the first FcRn antagonist for BP. In some embodiments, the BP180 antibody is an NC16A antibody.

In an embodiment, the reference value is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% higher than the lowest serum level of the BP180 antibody or the BP230 antibody measured in the subject following treatment with the first FcRn antagonist for BP. In some embodiments, the BP180 antibody is an NC16A antibody.

In an embodiment, the reference value is about 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, or 6.9-fold more than the lowest serum level of the BP180 antibody or the BP230 antibody measured in the subject following treatment with the first FcRn antagonist for BP. In some embodiments, the BP180 antibody is an NC16A antibody.

In an embodiment, the reference value is 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, or 6.9-fold more than the lowest serum level of the BP180 antibody or the BP230 antibody measured in the subject following treatment with the first FcRn antagonist for BP. In some embodiments, the BP180 antibody is an NC16A antibody.

In an embodiment, the subject was previously treated with a first FcRn antagonist at a dose of about 10 mg/kg to about 30 mg/kg, administered intravenously. In an embodiment, the subject was previously treated with a first FcRn antagonist at a dose of about 10 mg/kg, administered intravenously. In an embodiment, the subject was previously treated with a first FcRn antagonist at a dose of about 15 mg/kg, administered intravenously. In an embodiment, the subject was previously treated with a first FcRn antagonist at a dose of about 20 mg/kg, administered intravenously. In an embodiment, the subject was previously treated with a first FcRn antagonist at a dose of about 25 mg/kg, administered intravenously. In an embodiment, the subject was previously treated with a first FcRn antagonist at a dose of about 30 mg/kg, administered intravenously.

In an embodiment, the subject was previously treated with a first FcRn antagonist at a dose of about 10 mg/kg to about 30 mg/kg once weekly, administered intravenously. In an embodiment, the subject was previously treated with a first FcRn antagonist at a dose of about 10 mg/kg once weekly, administered intravenously. In an embodiment, the subject was previously treated with a first FcRn antagonist at a dose of about 15 mg/kg once weekly, administered intravenously. In an embodiment, the subject was previously treated with a first FcRn antagonist at a dose of about 20 mg/kg once weekly, administered intravenously. In an embodiment, the subject was previously treated with a first FcRn antagonist at a dose of about 25 mg/kg once weekly, administered intravenously. In an embodiment, the subject was previously treated with a first FcRn antagonist at a dose of about 30 mg/kg once weekly, administered intravenously.

In an embodiment, the subject was previously treated with a first FcRn antagonist at a dose of about 10 mg/kg to about 30 mg/kg once every two weeks, administered intravenously. In an embodiment, the subject was previously treated with a first FcRn antagonist at a dose of about 10 mg/kg once every two weeks, administered intravenously. In an embodiment, the subject was previously treated with a first FcRn antagonist at a dose of about 15 mg/kg once every two weeks, administered intravenously. In an embodiment, the subject was previously treated with a first FcRn antagonist at a dose of about 20 mg/kg once every two weeks, administered intravenously. In an embodiment, the subject was previously treated with a first FcRn antagonist at a dose of about 25 mg/kg once every two weeks, administered intravenously. In an embodiment, the subject was previously treated with a first FcRn antagonist at a dose of about 30 mg/kg once every two weeks, administered intravenously.

In an embodiment, the subject was previously treated with a first FcRn antagonist at a dose of about 750 mg to about 3000 mg, administered subcutaneously. In an embodiment, the subject was previously treated with a first FcRn antagonist at a dose of about 750 mg, administered subcutaneously. In an embodiment, the subject was previously treated with a first FcRn antagonist at a dose of about 1000 mg, administered subcutaneously. In an embodiment, the subject was previously treated with a first FcRn antagonist at a dose of about 1250 mg, administered subcutaneously. In an embodiment, the subject was previously treated with a first FcRn antagonist at a dose of about 1500 mg, administered subcutaneously. In an embodiment, the subject was previously treated with a first FcRn antagonist at a dose of about 1750 mg, administered subcutaneously. In an embodiment, the subject was previously treated with a first FcRn antagonist at a dose of about 2000 mg, administered subcutaneously.

In an embodiment, the subject was previously treated with a first FcRn antagonist at a dose of about 750 mg to about 3000 mg once weekly, administered subcutaneously. In an embodiment, the subject was previously treated with a first FcRn antagonist at a dose of about 750 mg once weekly, administered subcutaneously. In an embodiment, the subject was previously treated with a first FcRn antagonist at a dose of about 1000 mg once weekly, administered subcutaneously. In an embodiment, the subject was previously treated with a first FcRn antagonist at a dose of about 1250 mg once weekly, administered subcutaneously. In an embodiment, the subject was previously treated with a first FcRn antagonist at a dose of about 1500 mg once weekly, administered subcutaneously. In an embodiment, the subject was previously treated with a first FcRn antagonist at a dose of about 1750 mg once weekly, administered subcutaneously. In an embodiment, the subject was previously treated with a first FcRn antagonist at a dose of about 2000 mg once weekly, administered subcutaneously.

In an embodiment, the subject was previously treated with a first FcRn antagonist at a dose of about 750 mg to about 3000 mg once every two weeks, administered subcutaneously. In an embodiment, the subject was previously treated with a first FcRn antagonist at a dose of about 750 mg once every two weeks, administered subcutaneously. In an embodiment, the subject was previously treated with a first FcRn antagonist at a dose of about 1000 mg once every two weeks, administered subcutaneously. In an embodiment, the subject was previously treated with a first FcRn antagonist at a dose of about 1250 mg once every two weeks, administered subcutaneously. In an embodiment, the subject was previously treated with a first FcRn antagonist at a dose of about 1500 mg once every two weeks, administered subcutaneously. In an embodiment, the subject was previously treated with a first FcRn antagonist at a dose of about 1750 mg once every two weeks, administered subcutaneously. In an embodiment, the subject was previously treated with a first FcRn antagonist at a dose of about 2000 mg once every two weeks, administered subcutaneously.

In an embodiment, the subject was also previously treated with a corticosteroid or an immunosuppressive agent, such as dapsone. In an embodiment, the subject was previously treated with prednisone.

In an embodiment, the subject was previously treated with prednisone at a dose of 7.5 mg/day to 20 mg/day. In an embodiment, the subject was previously treated with prednisone at a dose of 8 mg/day to 15 mg/day. In an embodiment, the subject was previously treated with prednisone at a dose of 10 mg/day to 15 mg/day.

In an embodiment, the subject was previously treated with prednisone at a dose of ≤5 mg/kg/day. In an embodiment, the subject was previously treated with prednisone at a dose of ≤3 mg/kg/day. In an embodiment, the subject was previously treated with prednisone at a dose of ≤2 mg/kg/day. In an embodiment, the subject was previously treated with prednisone at a dose of ≤1 mg/kg/day. In an embodiment, the subject was previously treated with prednisone at a dose of ≤0.5 mg/kg/day. In an embodiment, the subject was previously treated with prednisone at a dose of ≤0.4 mg/kg/day. In an embodiment, the subject was previously treated with prednisone at a dose of ≤0.3 mg/kg/day. In an embodiment, the subject was previously treated with prednisone at a dose of ≤0.2 mg/kg/day. In an embodiment, the subject was previously treated with prednisone at a dose of ≤0.1 mg/kg/day.

In an embodiment, the effective amount of a second FcRn antagonist is a higher dose than the previous treatment with the first FcRn antagonist. In an embodiment, the effective amount of a second FcRn antagonist is a lower dose than the previous treatment with the first FcRn antagonist.

In an embodiment, the effective amount of the second FcRn antagonist is administered more frequently compared to the previous treatment with the first FcRn antagonist. In an embodiment, the effective amount of the second FcRn antagonist is administered less frequently compared to the previous treatment with the first FcRn antagonist.

In an embodiment, the effective amount of the second FcRn antagonist is administered intravenously at a dose of about 10 mg/kg to about 30 mg/kg once weekly, once every two weeks, once every three weeks, once every four weeks, once monthly, or once every six weeks. In an embodiment, the effective amount of the second FcRn antagonist is administered intravenously at a dose of 10 mg/kg once weekly, once every two weeks, once every three weeks, once every four weeks, once monthly, or once every six weeks. In an embodiment, the effective amount of the second FcRn antagonist is administered intravenously at a dose of 15 mg/kg once weekly, once every two weeks, once every three weeks, once every four weeks, once monthly, or once every six weeks. In an embodiment, the effective amount of the second FcRn antagonist is administered intravenously at a dose of 20 mg/kg once weekly, once every two weeks, once every three weeks, once every four weeks, once monthly, or once every six weeks. In an embodiment, the effective amount of the second FcRn antagonist is administered intravenously at a dose of 25 mg/kg once weekly, once every two weeks, once every three weeks, once every four weeks, once monthly, or once every six weeks. In an embodiment, the effective amount of the second FcRn antagonist is administered intravenously at a dose of 30 mg/kg once weekly, once every two weeks, once every three weeks, once every four weeks, once monthly, or once every six weeks.

In an embodiment, the effective amount of the second FcRn antagonist is administered intravenously at a dose of about 10 mg/kg once weekly, once every two weeks, once every three weeks, once every four weeks, once monthly, or once every six weeks. In an embodiment, the effective amount of the second FcRn antagonist is administered intravenously at a dose of about 15 mg/kg once weekly, once every two weeks, once every three weeks, once every four weeks, once monthly, or once every six weeks. In an embodiment, the effective amount of the second FcRn antagonist is administered intravenously at a dose of about 20 mg/kg once weekly, once every two weeks, once every three weeks, once every four weeks, once monthly, or once every six weeks. In an embodiment, the effective amount of the second FcRn antagonist is administered intravenously at a dose of about 25 mg/kg once weekly, once every two weeks, once every three weeks, once every four weeks, once monthly, or once every six weeks. In an embodiment, the effective amount of the second FcRn antagonist is administered intravenously at a dose of about 30 mg/kg once weekly, once every two weeks, once every three weeks, once every four weeks, once monthly, or once every six weeks.

In an embodiment, the effective amount of the second FcRn antagonist is administered subcutaneously at a fixed dose of about 750 mg to about 3000 mg once weekly, once every two weeks, once every three weeks, once every four weeks, once monthly, or once every six weeks. In an embodiment, the effective amount of the FcRn second antagonist is administered subcutaneously at a fixed dose of 1000 mg or 2000 mg once weekly, once every two weeks, once every three weeks, once every four weeks, once monthly, or once every six weeks. In an embodiment, the effective amount of the second FcRn antagonist is administered subcutaneously at a fixed dose of about 750 mg once weekly, once every two weeks, once every three weeks, once every four weeks, once monthly, or once every six weeks. In an embodiment, the effective amount of the second FcRn antagonist is administered subcutaneously at a fixed dose of about 1000 mg once weekly, once every two weeks, once every three weeks, once every four weeks, once monthly, or once every six weeks. In an embodiment, the effective amount of the second FcRn antagonist is administered subcutaneously at a fixed dose of about 1250 mg once weekly, once every two weeks, once every three weeks, once every four weeks, once monthly, or once every six weeks. In an embodiment, the effective amount of the second FcRn antagonist is administered subcutaneously at a fixed dose of about 1500 mg once weekly, once every two weeks, once every three weeks, once every four weeks, once monthly, or once every six weeks. In an embodiment, the effective amount of the second FcRn antagonist is administered subcutaneously at a fixed dose of about 1750 mg once weekly, once every two weeks, once every three weeks, once every four weeks, once monthly, or once every six weeks. In an embodiment, the effective amount of the second FcRn antagonist is administered subcutaneously at a fixed dose of about 2000 mg once weekly, once every two weeks, once every three weeks, once every four weeks, once monthly, or once every six weeks.

In an embodiment, the effective amount of the second FcRn antagonist is administered subcutaneously at a fixed dose of 750 mg once weekly, every two weeks, every three weeks, every four weeks, every month, or every six weeks. In an embodiment, the effective amount of the second FcRn antagonist is administered subcutaneously at a fixed dose of 1000 mg once weekly, every two weeks, every three weeks, every four weeks, every month, or every six weeks. In an embodiment, the effective amount of the second FcRn antagonist is administered subcutaneously at a fixed dose of 1250 mg once weekly, every two weeks, every three weeks, every four weeks, every month, or every six weeks. In an embodiment, the effective amount of the second FcRn antagonist is administered subcutaneously at a fixed dose of 1500 mg once weekly, every two weeks, every three weeks, every four weeks, every month, or every six weeks. In an embodiment, the effective amount of the second FcRn antagonist is administered subcutaneously at a fixed dose of 1750 mg once weekly, every two weeks, every three weeks, every four weeks, every month, or every six weeks. In an embodiment, the effective amount of the second FcRn antagonist is administered subcutaneously at a fixed dose of 2000 mg once weekly, every two weeks, every three weeks, every four weeks, every month, or every six weeks.

In an embodiment, the method further comprises administering to the subject an effective amount of a corticosteroid or an immunosuppressive agent. In an embodiment, the effective amount of the corticosteroid is administered at a dose of about 2 mg/kg/day. In an embodiment, the effective amount of the corticosteroid is administered at a dose of about 1 mg/kg/day. In an embodiment, the effective amount of the corticosteroid is administered at a dose of about 0.5 mg/kg/day. In an embodiment, the effective amount of the corticosteroid is administered at a dose of about 0.4 mg/kg/day. In an embodiment, the effective amount of the corticosteroid is administered at a dose of about 0.3 mg/kg/day. In an embodiment, the effective amount of the corticosteroid is administered at a dose of about 0.25 mg/kg/day. In an embodiment, the effective amount of the corticosteroid is administered at a dose of about 0.2 mg/kg/day. In an embodiment, the effective amount of the corticosteroid is administered at a dose of about 0.1 mg/kg/day. In an embodiment, the effective amount of the corticosteroid is administered at a dose of about 0.05 mg/kg/day.

In an embodiment, the effective amount of the corticosteroid is administered at a dose of about 20 mg/day. In an embodiment, the effective amount of the corticosteroid is administered at a dose of about 15 mg/day. In an embodiment, the effective amount of the corticosteroid is administered at a dose of about 10 mg/day. In an embodiment, the effective amount of the corticosteroid is administered at a dose of about 7.5 mg/day. In an embodiment, the effective amount of the corticosteroid is administered at a dose of about 5 mg/day. In an embodiment, the effective amount of the corticosteroid is administered at a dose of about 4 mg/day. In an embodiment, the effective amount of the corticosteroid is administered at a dose of about 3 mg/day. In an embodiment, the effective amount of the corticosteroid is administered at a dose of about 2 mg/day. In an embodiment, the effective amount of the corticosteroid is administered at a dose of about 1 mg/day. In an embodiment, the effective amount of the corticosteroid is administered at a dose of about 0.5 mg/day.

In an embodiment, the subject the corticosteroid dosing regimen is tapered based on the serum level of a BP180 antibody or BP230 antibody in a subject with BP. In some embodiments, the BP180 antibody is an NC16A antibody. In an embodiment, tapering the corticosteroid regimen is lowering the dose or lowering the dosing frequency of the corticosteroid. In an embodiment, the tapered corticosteroid dose is ≤2 mg prednisone/kg/day or equivalent OCS. In an embodiment, the tapered corticosteroid dose is less than or equal to about 1.5, 1.0, 0.75, 0.5, or 0.2 mg prednisone/kg/day or equivalent OCS. In an embodiment, the tapered corticosteroid dose is ≤0.5 mg prednisone/kg/day or equivalent OCS. In an embodiment, the tapered corticosteroid dose is ≤0.1 mg prednisone/kg/day or equivalent OCS.

In an embodiment, the serum level of the BP180 antibody or the BP230 antibody is measured by ELISA or addressable laser bead immunoassay (ALBIA). In some embodiments, the BP180 antibody is an NC16A antibody.

In an embodiment, the first FcRn antagonist and the second FcRn antagonist are each the same FcRn antagonist. In an embodiment, the first FcRn antagonist and the second FcRn antagonist are each a different FcRn antagonist.

In an embodiment, the FcRn antagonist is an Fc region comprising amino acids Y, T, E, K, F, and Y at EU positions 252, 254, 256, 433, 434, and 436, respectively. In an embodiment, the first FcRn antagonist or the second FcRn antagonist is an Fc region comprising amino acids Y, T, E, K, F, and Y at EU positions 252, 254, 256, 433, 434, and 436, respectively.

In an embodiment, the FcRn antagonist is efgartigimod. In an embodiment, the first FcRn antagonist or the second FcRn antagonist is efgartigimod.

In an embodiment, the FcRn antagonist comprises the amino acid sequence of SEQ ID NO: 1, 2, or 3. In an embodiment, the first FcRn antagonist or the second FcRn antagonist comprises the amino acid sequence of SEQ ID NO: 1, 2, or 3.

In an embodiment, the FcRn antagonist is an anti-FcRn antibody. In an embodiment, the first FcRn antagonist is an anti-FcRn antibody. In an embodiment, the second FcRn antagonist is an anti-FcRn antibody.

In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161).

In an embodiment, the first FcRn antagonist is an anti-FcRn antibody and the second FcRn antagonist is efgartigimod. In an embodiment, the first FcRn antagonist is an anti-FcRn antibody and the second FcRn antagonist comprises the amino acid sequence of SEQ ID NO: 1, 2, or 3. In an embodiment, the anti-FcRn antibody is rozanolixizumab (UCB7665), nipocalimab (M281), orilanolimab (ALXN1830/SYNT001), or batoclimab (IMVT-1401/RVT1401/HBM9161). In an embodiment, the patient has not been previously treated with efgartigimod.

In an embodiment, the first FcRn antagonist is selected from the group consisting of rozanolixizumab (UCB7665), nipocalimab (M281), orilanolimab (ALXN1830/SYNT001), or batoclimab (IMVT-1401/RVT1401/HBM9161) and the second FcRn antagonist is an Fc region comprising amino acids Y, T, E, K, F, and Y at EU positions 252, 254, 256, 433, 434, and 436, respectively. In an embodiment, the first FcRn antagonist is selected from the group consisting of rozanolixizumab (UCB7665), nipocalimab (M281), orilanolimab (ALXN1830/SYNT001), or batoclimab (IMVT-1401/RVT1401/HBM9161) and the second FcRn antagonist is efgartigimod.

### EXAMPLES

The following examples are offered by way of illustration, and not by way of limitation.

### Example 1: Efficacy of an Efgartigimod Analogue in an Acute Mouse Model of Bullous Pemphigoid

Bullous pemphigoid (BP) is an autoimmune blistering disease, most commonly observed in the elderly. BP patients have autoantibodies directed against two hemidesmosomal proteins, BP180 and BP230. Anti-BP180 autoantibodies mainly recognize the extracellular 16th non-collagenous domain of the molecule (NC16A). Animal model studies using humanized NC16A mice have demonstrated that anti-BP180 NC16A IgG are pathogenic (Liu et al., J Clin Invest. 1993; 92:2480-2488; Nishie et al., Nat Med. 2007; 13:378-383; Liu et al., J Autoimmun. 2008; 31:331-338). The aim of this study was to investigate the in vivo effect of an efgartigimod analogue used in mouse models (HEL-ABDEG) to inhibit BP disease in acute preventative and therapeutic BP mouse models. HEL-ABDEG is a full-length human IgG1 molecule comprising the Y, T, E, K, and F mutations at EU positions 252, 254, 256, 433, and 434 in the heavy chain.

### A. Methods

Serum samples were collected from 2 patients with active BP (BP1 and BP2). Patients had signed an informed consent and the samples were de-identified before transfer to the lab. These patients presented with generalized tense blisters and dermal-epidermal separation with inflammatory cell infiltration by routine histology. Direct immunofluorescence showed deposition of IgG at the basement membrane zone (BMZ) of perilesional skin. Indirect immunofluorescence (IIF) showed “roof staining” of salt-split human skin cryosections with IgG titers of 1:320 (BP1) and 1:640 (BP2) determined by IIF as described (Liu et al., J Autoimmun. 2008; 31:331-338).

hNC16A-specific total IgG were purified from BP patient sera using a protein G column, followed by an hNC16A-specific glutathione sepharose column as described (Liu et al., J Autoimmun. 2008; 31:331-338). The concentrations of purified IgG were quantified by human IgG-specific ELISA (Invitrogen, Cat #BMS2091). The purity of hNC16A-specific IgG was 96% (BP1) and 92% (BP2). Purified anti-hNC16A IgG fractions from BP1 and BP2 were concentrated by ultrafiltration (Millipore) stored frozen in PBS, pooled 1:1 and used for in vivo experiments.

Humanized NC16A mice were generated by UNC Animal Model Core as described by Liu et al. (J Autoimmun. 2008; 31:331-338). Briefly, to generate the humanized BP180 NC16A mice (hNC16A), the murine BP180 genomic segment encoding the NC14A domain (exons 17 through 18) was replaced by the human NC16A genomic sequence (exons 18 through 19). The targeting vector was electroporated into 129/Ola mouse ES cells. After the neo gene was excised from the targeted locus by Flp treatment, neo-minus cells were microinjected into C57BL/6J mouse blastocysts, which were then implanted into pseudopregnant recipients. High-chimera males were mated with B6 females to produce F1 heterozygotes (NC16A+/−). Crossing F1+/−produced F2 homozygotes expressing only the mhBP180NC16A hybrid antigen (NC16A+/+). The NC16A+/+ mice were back-crossed with C57BL/6J for >10 generations. C57BL/6J mice were obtained from Jackson Laboratories.

To test if HEL-ABDEG antibodies are effective to prevent onset of BP disease, adult (8-10 weeks old; n=8 per group) humanized NC16A (hNC16A) mice generated by the Liu lab (as described in Liu et al., J Autoimmun. 2008; 31:331-338) were injected intraperitoneally (i.p.) with pathogenic human anti-hNC16A IgG (250 μg/g body weight), immediately followed by intravenous (i.v.) injection with HEL-ABDEG (60 μg/g body weight) or PBS control. A third group of mice was injected with normal human control IgG (250 μg/g body weight), followed by PBS. The mice were examined 3 days post treatment. Clinical disease activity was scored on ear skin lesions (described below). After clinical examination, 2 animals in the PBS group and 3 animals in the HEL-ABDEG group were sacrificed, and skin lesion specimens were obtained for histopathology (described below) and IIF (described below).

To test if HEL-ABDEG antibodies are effective to treat established ongoing BP disease, adult (8-10 weeks old) hNC16A mice were injected i.p. with pathogenic anti-hNC16A IgG (250 μg/g body weight). Three days later, when mice developed clinical and histopathological signs of BP, the mice were treated i.v. with HEL-ABDEG (60 μg/g body weight) or PBS control and were examined on day 10 and 17 post anti-hNC16A IgG injection. Due to limited mice availability and gender repartition, 7 mice were used for HEL-ABDEG group and 9 mice for PBS control group. Two mice/group were sacrificed and skin lesion specimens were obtained for IIF.

Clinical disease score of the ear was measured as the sum of ear thickness+redness score+scaling score as used in Lin et al. (J Invest Dermatol. 2018; 138 (5): 1032-1043). A score of 0=normal (i.e., naïve ear); 1=mild; 2=moderate; 3=severe; 4=very severe.

Skin sections were used for routine histological examination by light microscopy (H/E staining) to localize the lesioned site (dermal-epidermal separation) and neutrophil infiltration. The disease activity in adult mice was scored based on dermal-epidermal separation by histology: (−)=no detectable dermal-epidermal separation; (1+)=mild inflammatory reaction and <25% epidermal detachment; (2+)=intense inflammatory reaction and epidermal detachment involving 25% to 50% of the epidermis; and (3+)=intense inflammatory reaction with frank epidermal detachment involving >50% of the epidermis.

For IIF, anti-hNC16A IgG (1 μg/mL) was incubated on skin sections of hNC16A mice. Binding of anti-hNC16A IgG to the BMZ was detected by AlexaFluor 594 conjugated goat anti-human IgG antibody (Life Technologies, catalog #A11014). To detect infiltrating neutrophils, skin sections were incubated with Ly6G (Biolegend, catalog #127602), followed by AlexaFluor 488 goat anti-rat (Life Technologies, catalog #A11006). Dapi was obtained from Sigma Aldrich (catalog #D9542).

All testing described above was conducted by experimenters blinded to treatments. Data were analyzed using the Student t-test with SigmaPlot software; a p value <0.05 was considered significant.

### B. Results

### HEL-ABDEG Treatment Abolishes BP Disease in an Acute Preventative BP Model

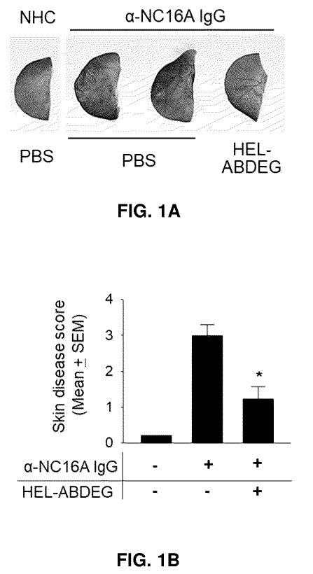

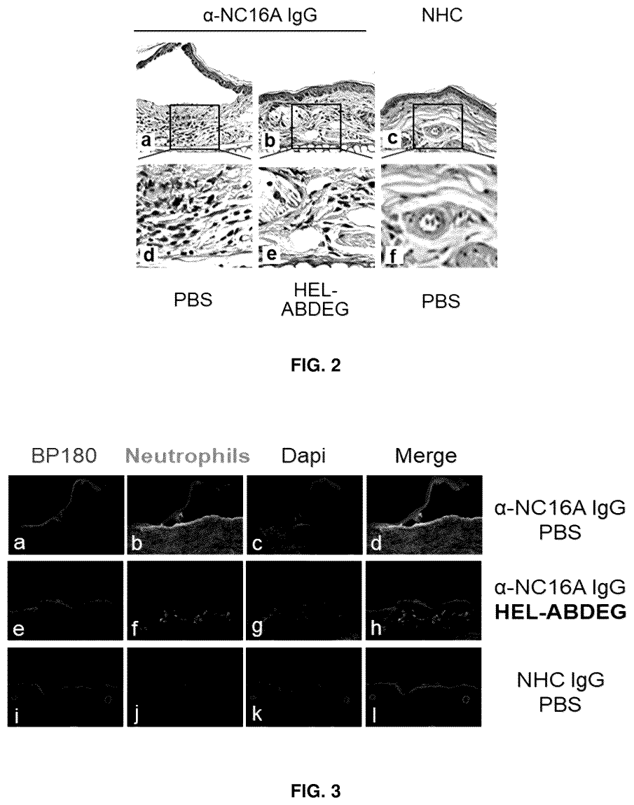

Mice injected with pathogenic anti-hNC16A IgG developed typical BP skin lesions, while mice injected with both pathogenic anti-hNC16A IgG and HEL-ABDEG had drastically reduced skin lesions as shown clinically (FIG. 1A-1B) and histologically (FIG. 2). The reduced BP disease activity caused by HEL-ABDEG treatment is associated with significantly decreased neutrophil infiltration in the skin (FIGS. 2 & 3).

### HEL-ABDEG Treatment Abolishes BP Disease in an Acute Therapeutic BP Model

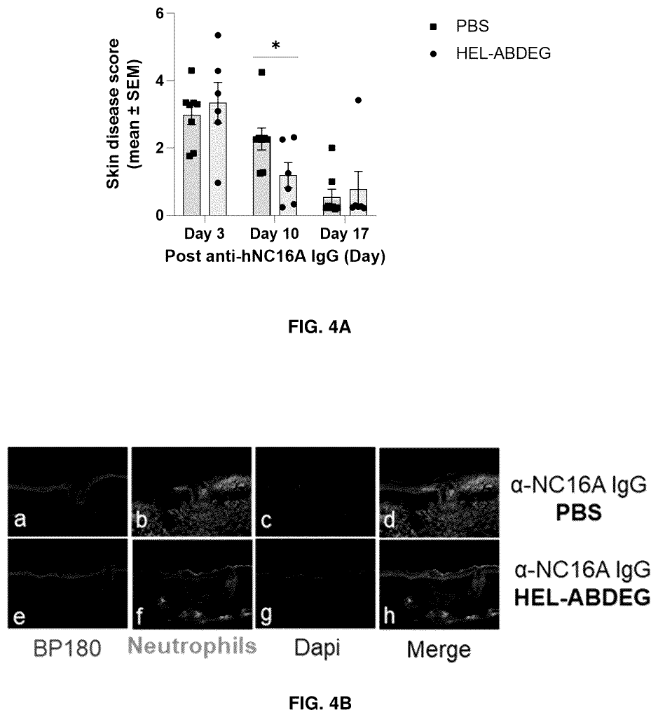

To test if HEL-ABDEG showed efficacy to treat established BP disease, mice were first injected i.p. with pathogenic anti-hNC16A IgG. Three days later, when the mice developed extensive BP disease, the mice were treated i.v. with HEL-ABDEG (60 μg/g body weight) or PBS control. Mice treated with HEL-ABDEG showed significantly reduced skin lesions on day 10 post anti-hNC16A IgG injection and less neutrophil infiltration as compared to PBS control mice (FIG. 4A-4B).

No clinical disease activity was seen at day 17 in both the HEL-ABDEG treated and the PBS-treated control mice. This can be explained by the fact that the pathogenic anti-hNC16A IgG are cleared and/or by immunogenicity against HEL-ABDEG (not tested). For this reason, neutrophil infiltration was not examined on day 17.

### C. Conclusions

Two acute mouse models were used to study the in vivo efficacy of HEL-ABDEG, an efgartigimod analogue, to inhibit anti-hNC16A IgG-induced BP disease. In the acute preventative BP disease model, HEL-ABDEG treated mice showed fewer skin lesions, both clinically (p<0.01) and histologically, with reduced neutrophil skin infiltration compared to control mice treated with PBS.

In the acute therapeutic BP disease model, mice were treated 3 days post anti-hNC16A IgG administration. HEL-ABDEG treated mice had a statistically significant reduction (p<0.05 compared with control) in disease activity on day 10, as well as less skin infiltrating neutrophils as evidenced by IIF compared with control. The disease-inducing effect of the pathogenic anti-hNC16A IgG antibodies is lost at day 17, which could be due to immunogenicity or the clearance of the anti-hNC16A IgG. Indeed, the half-life of human IgG in mice was previously reported to be 6-8 days. Thus, the results of this model have to be interpreted with caution at day 17. Nevertheless, therapeutic treatment with HEL-ABDEG reduced disease severity at day 10 in this model.

### Example 2: Phase 2/3 Study of Efgartigimod PH20 SC in Adult Participants with Bullous Pemphigoid (BALLAD)

BALLAD (ARGX-113-2009) is an operationally seamless 2-part, phase 2/3, prospective, global, multicenter, randomized, double blinded, placebo-controlled study to investigate the efficacy, safety, tolerability, immunogenicity, participant-reported outcome measures (including those assessing participant quality of life [QoL]), PK, and PD of efgartigimod PH20 SC (efgartigimod coformulated with recombinant human PH20 for subcutaneous (SC) injection) in adult participants with moderate-to-severe BP. This study intends to demonstrate that efgartigimod is an effective and safe treatment for BP, providing participants with control of disease activity (CDA) and eventually remission while reducing their cumulative exposure to OCS.

The study consists of 2 parts. Part A is a phase 2 evaluation to provide proof of concept for the therapeutic activity of efgartigimod PH20 SC in participants with BP. Part B is a phase 3 evaluation to confirm the results obtained from part A in a separate, larger group of participants with BP.

An interim analysis will be performed during part A (on data obtained through week 26 for all part A participants) to assess the primary endpoint and several secondary endpoints, confirm the appropriate sample size for part B of the study, and determine whether the efficacy results observed through week 26 of part A warrant continued study of efgartigimod PH20 SC for the treatment of participants with BP (futility analysis).

Other than differences in main goals, endpoints, and statistical analyses, parts A and B are identical in schedule, structure, assessments, and conduct.

To allow for convenient SC injection of investigational medicinal product (IMP), participants will be administered efgartigimod PH20 SC, a solution of efgartigimod coformulated with rHuPH20, or a matching placebo with the same concentration of rHuPH20. rHuPH20 is currently being used in coformulations with approved therapeutic antibodies to facilitate SC injection of volumes >2 mL. Efgartigimod, as both an IV formulation (efgartigimod IV) and efgartigimod PH20 SC, has been investigated in nonclinical studies, clinical studies of healthy volunteers, and clinical studies of patients with IgG-driven autoimmune diseases (generalized myasthenia gravis [gMG], primary immune thrombocytopenia [ITP], chronic inflammatory demyelinating polyneuropathy [CIPD], and pemphigus).

In clinical studies that included PD assessments, efgartigimod IV and efgartigimod PH20 SC have effectively reduced IgG antibodies. Available clinical data support the clinical benefit of efgartigimod. Efficacy was proven in a pivotal phase 3 study in participants with gMG (ARGX-113-1704), and clear signs of efficacy were demonstrated in phase 2 studies in participants with primary ITP (ARGX-113-1603) or pemphigus (ARGX-113-1701).

The primary objective of BALLAD is to evaluate the efficacy of efgartigimod PH20 SC for achieving sustained remission in the treatment of participants with BP.

### A. Study Design

### Overall Design

During both parts of the study, participants will be administered investigational medicinal product (IMP: efgartigimod PH20 SC or placebo [PBO PH20 SC]) once weekly for 36 weeks. All participants will also receive concurrent therapy with OCS, i.e., prednisone at a starting dosage of 0.5 mg/kg/day at baseline, or an alternate OCS at an equivalent dose strength. The OCS dosage will be adjusted according to each participant's BP disease status throughout the study.

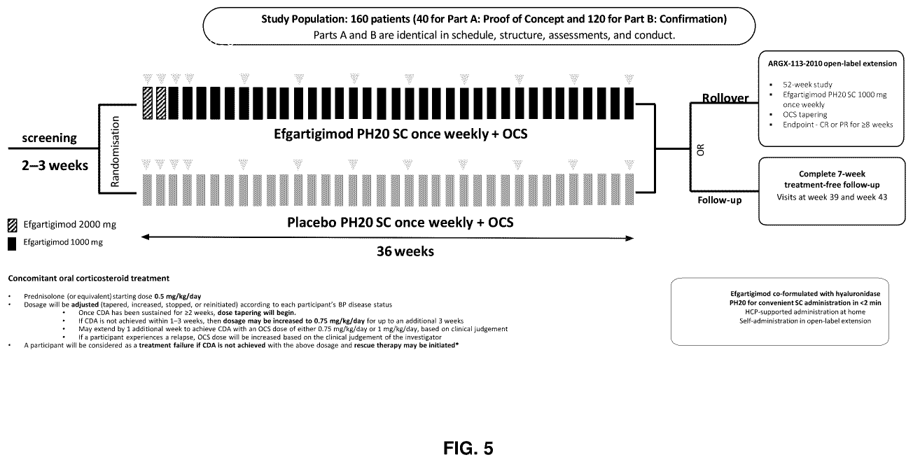

A schematic of the overall study designed is shown in FIG. 5. Brief descriptions of both parts of ARGX-113-2009 are presented below.

### Part A—Phase 2 Stage

Approximately 40 adult participants with moderate-to-severe BP will be randomly assigned to treatment with either efgartigimod PH20 SC or PBO PH20 SC. The main goal of part A is to determine whether efgartigimod PH20 SC is effective in the treatment of participants with BP via assessment of the study's primary endpoint: the proportion of participants who are in CR while receiving efgartigimod PH20 SC or placebo and have been off OCS therapy for >8 weeks at week 36. Other part A objectives and endpoints are described below.

An interim analysis will be conducted from data obtained from all part A participants through the week 26 visit. The interim analysis will be conducted to assess the primary endpoint, assess several of the study's secondary endpoints, confirm the appropriate sample size for part B of the study, and determine whether the efficacy results observed through week 26 warrant continued study of efgartigimod PH20 SC for the treatment of participants with BP (futility analysis).

The final analysis of part A data will be performed after its last participant completes their last visit.

### Part B—Phase 3 Stage

Part B is a phase 3, confirmatory study in which approximately 120 adult participants with moderate-to-severe BP will be randomly assigned to treatment with either efgartigimod PH20 SC or PBO PH20 SC. The main goal of part B is to confirm the results obtained for the primary endpoint from part A (the proportion of participants who are in CR while receiving efgartigimod PH20 SC or placebo and have been off OCS therapy for >8 weeks at week 36), but in a separate, larger group of participants with BP. Other part B objectives and endpoints are described below. The proposed order of the key secondary endpoints for part B may be adjusted based on the results obtained from the part A interim analysis.

### Conduct of Both Parts A and B

Parts A and B of the study have different sample sizes; otherwise, both parts are identical in schedule, structure, assessments, and conduct. Both parts A and B have screening periods of up to 2 weeks (extendable to 3 weeks per investigator discretion) followed by treatment periods of 36 weeks, during which participants will receive either efgartigimod PH20 SC or placebo (PBO PH20 SC). Finally, all part A and part B participants who complete the end of treatment period (EoTP) visit at week 36 will be offered the option to either enroll in an open-label extension (OLE) study or complete a 7-week treatment free follow up period (described in Example 3 below).

To enter the study, participants must have a diagnosis of BP that complies with the international guidelines for the diagnosis of BP. The diagnosis of BP must be confirmed by all three of the following methods before the participant is randomized to study intervention:
Clinical signs of BP (presence of bullae), with or without the presence of urticarial, eczematous, or erythematous plaques, and with or without pruritis.
Histopathological and direct immunofluorescence (DIF) examination of a cutaneous biopsy
Serology by indirect immunofluorescence (IIF), chemiluminescence enzyme immunoassay (CLEIA), or ELISA.

In part B, study participants will be stratified by disease history (newly diagnosed or relapsing) and disease severity (moderate BP [an Investigator Global Assessment of Bullous Pemphigoid {IGA-BP} score of 3] or severe BP [an IGA-BP score of 4]). The participants will be randomly assigned in a 1:1 ratio to receive either efgartigimod PH20 SC or PBO PH20 SC as follows:
Efgartigimod PH20 SC will be administered by SC injection on day 1 and day 8 at a dose of 2000 mg (administered as 2 separate SC injections of 1000 mg each), followed by single weekly SC injections of 1000 mg from week 2 through week 35.
PBO PH20 SC (injection vehicle co-formulated with 2000 U/mL of rHuPH20) will be administered using the same regimen described above.

All participants will also receive concurrent therapy with OCS, i.e., prednisone at a starting dosage of 0.5 mg/kg/day at baseline, or an alternate OCS at an equivalent dose strength. The OCS dosage will be adjusted according to each participant's BP disease status throughout the study. BP disease status terminology is defined elsewhere herein and the oral prednisone dose adjustment procedure is described below.

Efficacy and QoL measures to be assessed during the study (other than BP disease status assessment) include the following:
Investigator Global Assessment of Bullous Pemphigoid (IGA-BP)
Bullous Pemphigoid Disease Area Index (BPDAI)
Itch Numerical Rating Scale (Itch NRS)
EuroQOL 5 Dimension 5 Level (EQ-5D 5L)
Dermatology Life Quality Index (DLQI)
Autoimmune Bullous Disease Quality of Life (ABQOL) questionnaire.

The timepoints when these assessments are administered during the study are provided in the schedule of activities (SoA) (Table S4).

PK measures include the quantification of efgartigimod serum concentrations. PD measures include the quantification of total IgG serum levels, anti-BP180 and anti BP230 antibodies, and IgE serum levels. See the SoA (Table S4) for the timepoints when PK and PD blood samples are collected during the study.

Immunogenicity measures include the determination of both ADAs against efgartigimod in serum and antibodies against rHuPH20 in plasma. See the SoA (Table S4) for the timepoints when immunogenicity blood samples are collected during the study.

Safety measures for the study include the following:
Physical examinations
Height, body weight, and vital signs measurements
Electrocardiograms (ECGs)
Recording of medical, surgical, and vaccination histories
Clinical safety laboratory tests, including clinical chemistry, hematology, coagulation, urinalysis, and virus serology
Serum and urine pregnancy tests for women of childbearing potential (WOCBP) or a follicle stimulating hormone (FSH) test to confirm postmenopausal status
The Glucocorticoid Toxicity Index (GTI), consisting of the Composite Glucocorticoid Toxicity Index (C-GTI) and the Glucocorticoid Toxicity Index Specific List (GTI SL)
Recording of adverse events (AEs), serious adverse events (SAEs), and adverse events of special interest (AESIs).

In addition, selected study sites will participate in the following substudies:
Photography substudy: Site staff will take photographs of bullous lesions located at various anatomical regions of participants per the discretion of the investigator. As a guidance, timepoints of baseline, CDA, CR, and relapse are indicated; however, photographs may also be taken at intermediate timepoints.

Vaccination response substudy: This substudy will investigate the influence of efgartigimod PH20 SC treatment on historical protective antibody titers and humoral and/or cellular responses to vaccines received during the study among participants who provide additional consent to participate in this substudy.

### Rationale for Selection of the Participant Population

To ensure that the study population aligns with the demographics of the general population of patients with BP (which occurs predominantly in people >65 years of age), ARGX 113-2009 study participants will include males and females who are of legal adult age with no upper age limit.

The sponsor has designed the participant eligibility criteria of ARGX-113-2009 to comply with international guidelines for the diagnosis of BP which will ensure that the study population is representative of the general worldwide population of patients with BP. Bernard P et al., Am J Clin Dermatol. 2017; 389 (10079): 1630-1638; Witte M. et al., Front Med. 2018; 5:296; Di Lernia V et al., Derm Pract Concept. 2020; 10 (3): e2020050.

ARGX-113-2009 will compare the treatment group efgartigimod PH20 SC with PBO PH20 SC. All participants will receive concurrent therapy with oral corticosteroid (OCS), i.e., prednisone, at a starting dosage of 0.5 mg/kg/day at baseline or an alternate OCS at an equivalent dose strength. The OCS dosage will be adjusted according to each participant's BP disease status throughout the study.

ARGX-113-2009 will enroll participants with moderate-to-severe BP. This subgroup of patients was selected because it represents those patients in the general population who have the greatest medical need for new BP treatment modalities that can reduce their cumulative OCS exposure. Bernard P et al., Am J Clin Dermatol. 2017; 389 (10079): 1630-1638; Bernard P et al., Ann Dermatol Venereol. 2011; 138 (3): 247-251; Feliciani C et al., Br J Dermatol. 2015; 172 (4): 867-877.

The IGA-BP will be used to assess the severity of BP for each participant. It is a new assessment tool that was developed in 2021 by AIBD experts and is currently being used (along with the BPDAI) in other clinical studies of BP. The IGA-BP categorizes the severity of BP on a numerical scale of 0 to 4, with a score of 0 representing no overt signs of BP and a score of 4 representing severe BP. Given that this study is designed to investigate the effects of efgartigimod PH20 SC on participants with moderate-to-severe BP, ARGX-113-2009 will enroll participants whose IGA-BP score is 3 or 4.

Patients with BP can have comorbidities that may limit day-to-day functioning. Moreover, ARGX-113-2009 will not have an upper age limit for participation; therefore, the sponsor anticipates the study to include a large number of elderly participants with BP. Also, the sponsor wants to ensure that study participants will be able to adequately complete patient reported outcome (PRO) assessments without placing any undue burden on them. Therefore, ARGX-113-2009 participants will be required to have a Karnofsky Performance Index score of ≥60% (i.e., requires occasional assistance but is able to care for most needs or better).

Atypical types of BP and AIBDs other than BP may have pathogeneses and disease courses that differ from those of typical BP. Therefore, participants with other AIBDs or atypical forms of BP will be excluded from participating in the study.

Participants with BP who have been treated with any systemic therapies for the disease other than corticosteroids but still wish to participate in the study will be required to complete a wash out period before they can begin study participation. The durations of these required wash-out periods (described further below) were selected based on the known PK properties of each of these therapies. Prior therapy with conventional immunosuppressants (including but not limited to azathioprine, cyclophosphamide, methotrexate, and mycophenolate mofetil) or with dapsone require no wash out period; however, these therapies must be discontinued before baseline.

### Rationale for the Selection of the Oral Corticosteroid Dosing Regimen

The oral prednisone starting dosage of 0.5 mg/kg/day (or equivalent OCS) and the subsequent dosage modification regimen (described further below) are based on recommendations from European and Japanese guidelines. Schmidt E et al., J Dtsch Dermatol Ges. 2020; 18 (5): 516-526; Ujiie H et al., J Dermatol. 2019; 46 (12): 1102-1135. Oral prednisone doses will be adjusted throughout the study for each participant based on their current BP disease activity, with the aim to maintain CDA and to avoid relapse.

### Rationale for Primary Endpoint

The primary endpoint was chosen based upon the BP disease status terminology described by Murrell et al., J Amer Acad Dermatol. 2012; 66 (3): 479-485, in which the term CR off therapy is defined as “the absence of new or established lesions or pruritus while the patient is off all therapy for at least 2 months.” Both patients with BP and physicians who treat patients with the disease believe that achieving a complete and stable absence of BP symptoms represents a clinically meaningful outcome, resulting in a significant improvement in patient quality of life. Other than IMP, the only concurrent BP therapy permitted in ARGX-113-2009 is OCS; therefore, within the parameters of this study, “off OCS therapy” is considered equivalent to the AIBD expert definition of “off therapy.” In ARGX-113-2009, tapering of concurrent OCS therapy will be done in a manner consistent with treatment guidelines, while once-weekly dosing of IMP will be continued until the end of the 36-week treatment period.

Tapering, and ultimately eliminating, concurrent OCS therapy reduces the risks of developing comorbidities associated with steroid therapy. Avoiding such risks is particularly important for elderly patients (like those with BP), who historically develop serious comorbidities like osteoporosis, diabetes, and glaucoma following prolonged exposure to high doses of OCS. Joly P et al., N Engl J Med. 2002; 346 (5): 321-327; Hull C M et al., Arch Dermatol. 2003; 139 (2): 225-226; Bernard P et al., Ann Dermatol Venereol. 2011; 138 (3): 173-181.

This study will assess whether participants have remained in CR while receiving efgartigimod PH20 SC or placebo and after having been off OCS for at least 8 weeks at the end of the 36-week treatment period. Achieving a sustained response to treatment is of particular importance to patients with BP, as the available evidence shows that many patients who achieve CR subsequently relapse within months. Bernard P et al., Arch Dermatol. 2009; 145 (5): 537-542; Wang Y et al., Ann Med. 2018; 50 (3): 234-239.

The selection of a 36-week treatment period is based on international guidelines for treatment of BP, which recommend that systemic steroid doses should be tapered gradually so that minimal OCS therapy is achieved within 4-6 months after beginning treatment, and that OCS therapy may be subsequently stopped after the patient has sustained CR for 3-6 months. Therefore, a treatment period of 36 weeks (approximately 9 months) is considered an appropriate duration of time to allow patients to achieve CR and stop concurrent OCS therapy.

### Rationale for Key Secondary Endpoints

Rationales for the selection of the key secondary efficacy endpoints for part B are summarized below. [Note: These endpoints are “key” secondary endpoints (meaning that they are subject to hierarchical testing and alpha control) during part B only. During part A of the study, they will be assessed as standard secondary efficacy endpoints (without hierarchical testing/alpha control).]

Cumulative dose of OCS from baseline to week 36: Currently, the standard of care for moderate-to-severe BP is OCS. Unfortunately, exposure to high doses of OCS results in an increased risk of developing serious comorbidities, especially in elderly people. As a result, a goal in BP treatment is to reduce the patient's cumulative exposure to OCS. Therefore, this endpoint was selected to ascertain whether participants who are administered efgartigimod PH20 SC have lower cumulative OCS exposure values than participants who are administered PBO PH20 SC.

Proportion of participants who achieve an IGA-BP score of 0 while receiving efgartigimod PH20 SC or placebo and have been off OCS therapy for >8 weeks at week 36: This endpoint was selected because an IGA-BP score of 0 represents a complete absence of BP skin lesions. The ability to remain free of clinical disease activity while limiting one's cumulative exposure to OCS for prolonged periods is particularly important among elderly patients with BP, as they are prone to suffer from serious comorbidities associated with prolonged OCS exposure (e.g., osteoporosis, diabetes, glaucoma, etc.).

Proportion of participants who achieve CDA while receiving efgartigimod PH20 SC or placebo and remain free of relapse through week 36: This endpoint was selected because it is important to ascertain whether treatment with efgartigimod PH20 SC provides participants with BP prolonged and sustained relief from BP symptoms, and whether efgartigimod PH20 SC effectively prevents relapse.

Proportion of participants who are in CR while receiving efgartigimod PH20 SC or placebo and have been receiving minimal OCS therapy for >8 weeks at week 36: The ability to remain free of clinical disease activity while limiting one's cumulative exposure to OCS for prolonged periods is particularly important among elderly patients with BP, as they are prone to suffer from serious comorbidities associated with prolonged OCS exposure (e.g., osteoporosis, diabetes, glaucoma, etc.).

Changes from baseline in the 24-hour average itch score from the Itch NRS: Pruritus is among the signs/symptoms of BP; moreover, the itching that is associated with BP can be debilitating. This endpoint will determine if treatment with efgartigimod PH20 SC provides relief from pruritus, which is considered a clinically meaningful disease improvement.

### Rationale for Selection of Study Assessments

The BP disease assessment terminology and definitions applied in this study were adapted from those established by Murrell et al., J Amer Acad Dermatol. 2012; 66 (3): 479-485. These terms and definitions are internationally accepted by AIBD medical experts and clinicians who treat patients with BP.

Disease severity and symptoms will be monitored throughout the study by means of the IGA-BP scale, the BPDAI, and the Itch NRS. The IGA-BP was developed in 2021 by medical experts in the field of AIBDs, and it is currently being used (along with the BPDAI) in other clinical studies of therapies for BP. The IGA-BP scale has specific guidance on equating the extent of skin lesions to disease severity, while the BPDAI score also takes into account the presence of mucosal lesions. Pruritic symptoms will be measured using the Itch NRS.

The BPDAI and the Itch NRS are validated and internationally accepted measures of BP disease severity and pruritis intensity, respectively. Masmoudi W et al., Br J Dermatol. 2021; 184 (6): 1106-1112; Phan N et al., Acta Derm Venereol. 2012; 92 (5): 502-507; Verweyen E et al., Acta Derm Venereol. 2019; 99 (7): 657-663; Storck M et al., J Eur Acad Dermatol Venereol. 2021; 35 (5): 1176-1185.

The EQ-5D-5L, DLQI, and ABQOL are validated and internationally accepted measures of patient QOL. Finlay A Y et al., Clin Exp Dermatol. 1994; 19 (3) 210-216; Sebaratnam D F et al., JAMA Dermatol. 2013; 149 (10): 1186-1191.

### Rationale for Selection of the Treatment Regimen and Dose

In ARGX-113-2009, study participants will either receive efgartigimod PH20 SC or PBO PH20 subcutaneously (SC). To date, efgartigimod PH20 SC has been safely administered to healthy volunteers and participants with gMG, primary ITP, CIDP, and pemphigus.

The SC route of administration offers convenience for participants with BP, their caregivers, and healthcare providers, because SC injections are easier to administer than IV injections. Additionally, the coformulation of efgartigimod with rHuPH20 permits SC dosing of higher volumes than typical SC injections, because rHuPH20 reduces resistance to fluid flow and increases dispersion and absorption of injected medicines and fluids, which allows for a larger volume to be injected with limited skin swelling or pain.

In BP, patients typically achieve CDA, PR, or CR only after receiving treatment for several weeks; sometimes even months of treatment are required before CR or PR is achieved. Moreover, patients with BP tend to relapse within a few months after achieving remission. Bernard P et al., Arch Dermatol. 2009; 145 (5): 537-542; Wang Y et al., Ann Med. 2018; 50 (3): 234-239. Therefore, studies of BP must be of sufficient duration to assess the durability of efficacy of the IMP and the sustainability of remission while the patient is off OCS therapy. It is for these reasons that a 36-week treatment period has been selected for ARGX-113-2009.

Initially, the clinical development of efgartigimod was based on IV dosing. Results from phase 1 studies in healthy subjects, phase 2/3 studies in participants with gMG, a phase 2 study in participants with ITP, and PK/PD modeling analysis showed that efgartigimod IV 10 mg/kg administered once weekly achieved nearly maximal reduction in serum IgG levels. Studies of participants with gMG and ITP also showed that efgartigimod IV 10 mg/kg administered once weekly resulted in clinical efficacy coupled with reductions in serum pathogenic autoantibody levels while maintaining a favorable safety/tolerability profile.

In ARGX-113-2009, participants will be administered efgartigimod PH20 SC as a flat dose (rather than a dose that is adjusted weekly based on body mass), because this is more convenient for both the study participants and site staff. Results from a PK/PD modeling analysis indicated that efgartigimod PH20 SC 1000 mg administered once weekly would result in reductions in total IgG serum levels equivalent to a weekly dose of efgartigimod IV 10 mg/kg. Results from a phase 1 study comparing the PK and PD properties of IV and SC administration of efgartigimod in healthy subjects (ARGX-113-1907) confirmed that 4 once-weekly injections of efgartigimod PH20 SC 1000 mg was noninferior to 4 once-weekly infusions of efgartigimod IV 10 mg/kg in reducing total IgG serum levels. Therefore, the sponsor has selected efgartigimod PH20 SC 1000 mg administered once weekly as the maintenance dose for ARGX-113-2009.

Still, given that IgG autoimmune activity is considered to cause the pathophysiology of BP, any therapy that targets this activity should be designed so that maximum reduction of total IgG serum levels occurs as rapidly as possible. Therefore, ARGX-113-2009 participants who are assigned to the efgartigimod PH20 SC group will receive a dose of 2000 mg at baseline (week 0) and week 1. Initiation of treatment with 2 once-weekly doses of efgartigimod PH20 SC 2000 mg is predicted to achieve a nearly maximal reduction in total IgG serum levels within 2 weeks that is similar to that for once-weekly injections of efgartigimod PH20 SC 1000 mg at steady state.

### B. Study Population

Prospective approval of protocol deviations to recruitment and enrollment criteria, also known as protocol waivers or exemptions, is not permitted.

### Inclusion Criteria

Participants are eligible to be included in the study only if all of the following criteria apply:
Participant is willing and able to do the following: a) understand the requirements of the study; b) provide written informed consent (including consent for the use and disclosure of research-related health information); c) comply with the study protocol procedures (including required study visits);
The participant is male or female and has reached the age of consent at the time of signing the informed consent;
Participant has clinical signs of BP (i.e., presence of bullae), with or without the presence of urticarial/eczematous/erythematous plaques or pruritus at the screening and baseline visits. The diagnosis of BP must be confirmed by positive histopathology and DIF before randomization to treatment assignment, and by positive serology (by IIF, CLEIA, or ELISA, according to local practice) at screening;
Participant has an IGA-BP score of 3 or 4 at screening and baseline;
Participant has a Karnofsky performance status of at least 60% at screening;
Contraceptive use by men and women should be consistent with local regulations regarding the methods of contraception for those participating in clinical studies and:
   a. Male participants must agree to use an acceptable method of contraception from the time that the informed consent form is signed until the date of their last dose of IMP.
   b. Women of childbearing potential must have a negative serum pregnancy test at screening and a negative urine pregnancy test at baseline before study intervention can be administered; and must agree to use at least one contraception method from the time that the informed consent form is signed until the date of their last dose of IMP (a woman is considered to be of childbearing potential unless she is either postmenopausal or surgically sterilized).

### Exclusion Criteria

Participants are excluded from the study if any of the following criteria apply:
Other forms of pemphigoid (including but not limited to pemphigoid gestationis, drug induced BP that resolves after culprit-drug withdrawal, anti-p200 pemphigoid, mu-cous membrane pemphigoid, and cicatricial pemphigoid), or other AIBDs (including but not limited to epidermolysis bullosa acquisita, pemphigus vulgaris, and exfoliative erythroderma);
Received unstable dose of treatments known to cause or exacerbate BP (e.g., angiotensin converting enzyme inhibitors, penicillamine, furosemide, phenacetin, dipeptidyl peptidase 4 inhibitor) for at least 4 weeks prior to the baseline visit;
Use of BP treatments other than OCS, topical corticosteroids, conventional immunosuppressants (e.g., azathioprine, cyclophosphamide, methotrexate, mycophenolate, or mofetil), or dapsone, including the following:
   sulfasalazine, IVIg, subcutaneous administration of immunoglobulin (SCIg), immunoadsorption or plasma exchange within 8 weeks of the baseline visit;
   tetracyclines with or without nicotinamide at doses higher than the recommended daily allowance (RDA)/dietary reference intake (DRI) within 2 weeks of the baseline visit;
   any monoclonal antibody (including rituximab or another anti-CD20 biologic) within 6 months of the baseline visit;
   complementary therapies—such as traditional Chinese medicines, herbs, or procedures (e.g., acupuncture)—within 4 weeks (or 5 half-lives) of the baseline visit, if the investigator determines that such therapies may interfere with the study's efficacy assessments and/or potentially risk the safety of the participant;
Known contraindication to OCS therapy;
Clinically significant uncontrolled active or chronic, bacterial, viral, or fungal infection at screening;
Positive COVID-19 test result at screening (testing performed according to local requirements);
Any other known autoimmune disease that, in the opinion of the investigator, would interfere with an accurate assessment of clinical symptoms of BP or put the participant at undue risk;
History of malignancy unless deemed cured by adequate treatment with no evidence of recurrence for >3 years before the first administration of the IMP. Participants with the following cancers can be included at any time, provided they are adequately treated prior to their participation in the study: a) Basal cell or squamous cell skin cancer; b) Carcinoma in situ of the cervix; c) Carcinoma in situ of the breast; d) Incidental histological finding of prostate cancer (tumor-node metastasis [TNM] stage T1a or T1b);
Clinical evidence of other significant serious diseases, have had a recent surgery, or who have any other condition that, in the opinion of the investigator, could confound the results of the study or put the patient at undue risk;
Use of an investigational product within 3 months or 5 half-lives (whichever is longer) before the first dose of IMP;
Previously participated in a clinical study with efgartigimod;
Known hypersensitivity to any of the components of the administered treatments;
Positive serum test at screening for an active infection with any of the following conditions: a) HBV that is indicative of an acute or chronic infection, unless associated with a negative HbsAg or negative HBV DNA test; b) HCV based on HCV antibody assay unless a negative RNA test is available; c) HIV based on test results that are associated with an AIDS-defining condition or a CD4 count≤200 cells/mm³;
Primary or secondary hypogammaglobulinemia with total IgG serum levels <4 g/L at screening. Study candidates with total IgG serum levels of 4-6 g/L at screening may be permitted to participate in the study following discussion and agreement between the investigator and the sponsor.
Current or history (i.e., within 12 months of screening) of alcohol, drug, or medication abuse;
Pregnant or lactating females and those who intend to become pregnant during the study or within 90 days after last dose of the IMP;
Prolonged QTcF interval on ECG.

As described above, participants with BP who have been treated with any of the following will be excluded from study participation: a) Sulfasalazine, IVIg, SCIg, immunoadsorption, or plasma exchange within 8 weeks of the baseline visit; b) Tetracyclines (with nicotinamide [at doses higher than the RDA/DRI] or without nicotinamide) within 2 weeks of the baseline visit; c) Any monoclonal antibody (including rituximab or another anti-CD20 biologic) within 6 months of the baseline visit.

However, any potential ARGX-113-2009 candidate who has been treated with any of the above noted therapies and still wishes to participate in the study must complete a wash out period before they can be randomly assigned to study intervention. The duration of the required wash-out period must match the duration identified above for each class of medication. Note: therapy with topical corticosteroids and/or conventional immunosuppressants (including but not limited to azathioprine, cyclophosphamide, methotrexate, and mycophenolate mofetil) or with dapsone requires no wash-out period; however, these therapies must be discontinued before baseline.

### C. Study Intervention(s) and Concomitant Therapy

A list of study interventions (both IMPs and non-investigational medicinal products) is presented in Table S1, while the characteristics of the 2 treatment arms of ARGX-113-2009 are described in Table S2. Fixed doses of efgartigimod PH20 SC or PBO PH20 SC will be administered at body sites spared of any cutaneous BP lesions, with the abdominal area being the preferred site. Optional sites (such as thighs and arms) may be used. PBO PH20 SC will contain the same excipients as efgartigimod PH20 SC but without the active ingredient efgartigimod. Masked vials and masked syringes will be provided to preserve the study blind.

### Study Intervention(s) Administered

TABLE S1
Study intervention(s) administered

| Intervention | EFG PH20 SC | PBO PH20 SC | Oral Prednisone^{a} |
|---|---|---|---|
| Type | Biologic | Other: PBO | Non-IMP |
| Dose | EFG 180 mg/mL + | Vehicle + 2000 U/mL | Prednisone (or equivalent) |
| Formulation | 2000 U/mL rHuPH20 | rHuPH20 for SC injection | tablets for oral administration |
| | solution for SC injection to | | |
| | be administered at a fixed | | |
| | EFG dose of 1000 mg per | | |
| | injection | | |
| Unit Dose | 180 mg/mL | PBO | Prednisone (or equivalent) 2.5 |
| Strength(s) | | | mg, 5 mg, 10 mg, 20 mg |
| Dosage Level(s) | 2 × 1000 mg SC doses | 2 SC doses administered at | See below for oral prednisone |
| | administered at separate | separate sites on day 1 and | dosage taper/increase schedule |
| | sites (i.e., 2000 mg in total) | day 8; single SC doses | |
| | on day 1 and day 8; single | administered weekly from | |
| | 1000 mg SC doses | week 2 through week 35 | |
| | administered weekly from | | |
| | week 2 through week 35 | | |
| Route of | Abdominal SC injection(s); | Abdominal SC injection(s); | Oral administration |
| Administration | preferred site^{b} | preferred site^{b} | |
| Use | Investigational drug | PBO control | Non-IMP; concurrent therapy |
| | | for investigational drug | |
| IMP | IMP | IMP | Non-IMP |
| Sourcing | Provided by the sponsor to | Provided by the sponsor to | Provided by the sponsor to the |
| | the study site | the study site | study site. |
| | | | Local sourcing is permitted |
| | | | only if no doses are available at |
| | | | the site and only after |
| | | | consultation with the sponsor |
| | | | (or designee) |
| Packaging and | Provided in glass vials. | Provided in glass vials. | Non-IMP will be provided as 1 |
| Labeling | Each glass vial will be | Each glass vial will be | of the following: |
| | labeled as required per | labeled as required per | In the commercial package |
| | country requirement | country requirement | and labeled as required per |
| | | | country requirements, or |
| | | | As a magistral preparation |
| | | | upon prescription by the |
| | | | investigator, or |
| | | | In the commercial package |
| | | | sourced locally |

BP = bullous pemphigoid;
EFG = efgartigimod;
EFG PH20 SC = efgartigimod with rHuPH20 (coformulation) for SC injection;
IMP = investigational medicinal product;
OCS = oral corticosteroid (prednisone or equivalent);
PBO = placebo;
PBO PH20 SC = placebo with rHuPH20 (coformulation) for SC injection;
rHuPH20 = recombinant human hyaluronidase PH20;
SC = subcutaneous(ly)
^{a}Refers to oral prednisone or an equivalent OCS.
^{b}IMP will be administered on body sites spared of any cutaneous BP lesions; the abdomen is the preferred site. Optional sites (such as thighs or arms) may be chosen.

TABLE S2
Treatment arm characteristics

| Arm Title | EFG PH20 SC | PBO PH20 SC |
|---|---|---|
| Arm Type | Experimental | PBO |
| Arm Description | Participants will receive EFG PH20 SC | Participants will receive PBO PH20 SC (via |
| | 2000 mg (via 2 separate 1000 mg SC | 2 separate SC injections) on day 1 |
| | injections) on day 1 and day 8. | and day 8. |
| | Beginning at week 2, participants will | Beginning at week 2, participants will |
| | receive EFG PH20 SC 1000 mg (via single SC | receive PBO PH20 SC (via single SC |
| | injection) once weekly through week 35. | injection) once weekly through week 35. |
| | Participants will be administered | Participants will be administered |
| | concurrent OCS (oral prednisone) at 0.5 | concurrent OCS (oral prednisone) at 0.5 |
| | mg/kg/day, (or equivalent OCS) beginning | mg/kg/day (or equivalent OCS) beginning |
| | at baseline (week 0); this dosage will be | at baseline (week 0); this dosage will be |
| | adjusted throughout the study based on the | adjusted throughout the study based on the |
| | participant's current BP disease status. | participant's current BP disease status. |
| Associated Labels | EFG PH20 SC | PBO PH20 SC |
| | OCS | OCS |

BP = bullous pemphigoid;
EFG = efgartigimod;
EFG PH20 SC = efgartigimod with rHuPH20 (coformulation) for SC injection;
IMP = investigational medicinal product;
OCS = oral corticosteroid (prednisone or equivalent);
PBO = placebo;
PBO PH20 SC = placebo with rHuPH20 (coformulation) for SC injection;
rHuPH20 = recombinant human hyaluronidase PH20;
SC = subcutaneous(ly)

Any medication or vaccine (including over the counter or prescription medicines, recreational drugs, vitamins, and/or herbal supplements) that the participant is receiving at the time of screening or receives during the study must be recorded and include the following information: 1) Reason for use; 2) Dates of administration including start and end dates; 3) Dosage information including dose and frequency; 4) Brand name (for vaccines only).

All available vaccination history should be recorded as part of the participant's prior medication for vaccinations received in the past or as concomitant medication for vaccinations received during the trial. In addition, any vaccination information that the participant, their caregiver, or their legally authorized representative can remember should be recorded (with the brand name of the vaccine and date of vaccination, if possible).

The medical monitor should be contacted if there are any questions regarding concomitant or prior therapy.

All participants, regardless of study IMP assignment, will also receive concurrent therapy with OCS, i.e., prednisone at a starting dosage of 0.5 mg/kg/day at baseline (week 0), or an alternate OCS at an equivalent dose strength. The OCS dosage will be adjusted according to each participant's BP disease status throughout the study, with the goal of rapidly tapering systemic corticosteroid exposure and preventing and/or treating relapse. The oral prednisone dose adjustment procedure is described further below.

If the investigator deems it necessary, oral prednisone ≤0.3 mg/kg/day (or equivalent OCS) may be administered to participants during the period between their screening and baseline visits.

Information on the formulation, unit dose strength, sourcing, packaging, and labeling of oral prednisone is summarized in Table S1.

Investigators, study site staff, and participants must comply with package labeling instructions for proper oral prednisone storage and handling.

During each on site visit, study site staff will provide the participant with a supply of daily oral prednisone doses (or equivalent OCS) sufficient to last until the participant's next on-site visit. Participants will return any used and unused doses to the study center at their next on-site visit.

Participants will take oral prednisone (or equivalent OCS) daily at the dose directed by the investigator.

Participants will be provided a diary to record daily intake of each oral prednisone dose. The participant will bring their completed diary with them to every on-site visit so that staff can review the dosing record; during home visits, the participant will share their diary information with site staff.

If a participant misses one or more oral prednisone doses, they must contact the investigator. The missed doses must not be “made up”; rather, the participant will resume taking the daily oral prednisone dosage prescribed by the investigator, unless they have had a change in BP disease status that warrants dose adjustment.

The investigator should not deviate from the recommended concurrent oral prednisone dosing regimen (described below), except in matters where the safety of the participant may be compromised. After taking appropriate action to ensure the safety of the participants, such concerns should be documented and communicated to the sponsor's medical monitor and designee within 24 hours of the event. Note: For the following list, “oral prednisone” refers to prednisone or an alternate OCS of equivalent dose strength.

All participants will begin the study by taking oral prednisone 0.5 mg/kg/day starting at baseline (week 0; day 1).

If the participant has not achieved CDA within 1-3 weeks of treatment with oral prednisone 0.5 mg/kg/day, then the dosage may be increased to 0.75 mg/kg/day for up to an additional 3 weeks. This period may be extended by 1 additional week (based on clinical judgment) to achieve CDA with an oral prednisone dosage of either 0.75 mg/kg/day or 1 mg/kg/day.

A participant will be considered as a treatment failure if they do not achieve CDA after receiving the prednisone dosing regimen described above.

A participant who is a treatment failure will permanently discontinue study intervention, complete an ETD visit (Table S4), may begin rescue therapy (described below), and then continue participating in the study, resuming their treatment period visits as scheduled. Under these circumstances, the participant will be considered a non-responder.

When CDA is achieved, the oral prednisone dose regimen is adjusted as follows:
After CDA has been sustained for ≥2 weeks, the dose tapering schedule begins with the next lower oral prednisone dosage as listed in Table S3. Each new dosage must be maintained for at least 2 weeks provided that no new lesions (transient or non-transient) appear. Duration of tapering steps from escalated doses (>0.5 mg/kg/day) may be shortened based on clinical judgment.
For participants who achieved CDA on oral prednisone 0.5 mg/kg/day, the tapering steps are 0.3, 0.2, 0.15, and 0.1 mg/kg/day. For participants who required a dose increase to 0.75 or 1.0 mg/kg/day to achieve CDA, the oral prednisone dosage must first be tapered down to 0.5 mg/kg/day before proceeding to 0.3, 0.2, 0.15, and 0.1 mg/kg/day as shown in Table S3
Further tapering below the level of minimal OCS therapy can be performed in steps of 2.5 mg/day until the participant is off oral prednisone therapy (Table S3). Each new dosage step must be maintained for at least 2 weeks, provided that no new lesions (transient or non-transient) appear.
If a participant has new lesions but the investigator determines that it is not a relapse, prednisone tapering will be delayed. Under these circumstances, tapering can resume when no new lesions have appeared for at least 1 week (Table S3).

If a participant relapses:
The oral prednisone dosage is increased based on the clinical judgment of the investigator, with the recommendation to return to the dose administered 2 tapering steps before the relapse. Tapering can be resumed after CDA has been sustained for at least 2 weeks on this increased dose.
If CDA is not achieved, the participant should resume the initial oral prednisone dosage of 0.5 mg/kg/day.
The participant must resume weekly on-site visits until CDA returns.

During home visits that occur between mandatory on-site visits, the investigator will call the participant once weekly to review the participant's BP disease status and current oral prednisone dosing regimen.

TABLE S3
Concurrent oral prednisone equivalent doses (in mg) based on participant body weight

| | Body Weight Category^{a} | | | | | | | | | | | | | |
|---|---|---|---|---|---|---|---|---|---|---|---|---|---|---|
| Oral | 45 kg | 50 kg | 55 kg | 60 kg | 65 kg | 70 kg | 75 kg | 80 kg | 85 kg | 90 kg | 95 kg | 100 kg | 105 kg | |
| Prednisone | ≥45 | ≥50 | ≥55 | ≥60 | ≥65 | ≥70 | ≥75 | ≥80 | ≥85 | ≥90 | ≥95 | ≥100 | ≥105 | Tapering |
| Dose Level^{b} | to <50 | to <55 | to <60 | to <65 | to <70 | to <75 | to <80 | to <85 | to <90 | to <95 | to <100 | to <105 | to <110 | Frequency |
| mg/kg/day | mg/day | | | | | | | | | | | | | |
| 1.0^{c} | 45 | 50 | 55 | 60 | 65 | 70 | 75 | 80 | 85 | 90 | 95 | 100 | 105 | Every |
| 0.75^{c} | 30 | 35 | 40 | 45 | 45 | 50 | 55 | 60 | 60 | 65 | 70 | 75 | 75 | 2 weeks, |
| Starting | 22.5 | 25 | 25 | 30 | 30 | 35 | 35 | 40 | 40 | 45 | 45 | 50 | 50 | provided |
| dose: 0.50 | | | | | | | | | | | | | | that |
| 0.30 | 12.5 | 15 | 15 | 17.5 | 17.5 | 20 | 22.5 | 22.5 | 25 | 25 | 25 | 30 | 30 | no new |
| 0.20 | 7.5 | 10 | 10 | 10 | 12.5 | 12.5 | 15 | 15 | 15 | 17.5 | 17.5 | 20 | 20 | lesions |
| 0.15 | 5 | 7.5 | 7.5 | 7.5 | 7.5 | 10 | 10 | 10 | 12.5 | 12.5 | 12.5 | 15 | 15 | appear^{b} |
| Minimal | 2.5 | 5 | 5 | 5 | 5 | 5 | 7.5 | 7.5 | 7.5 | 7.5 | 7.5 | 10 | 10 | |
| OCS | | | | | | | | | | | | | | |
| ther- | | | | | | | | | | | | | | |
| apy: ≤0.10 | | | | | | | | | | | | | | |
| mg/day | mg/day | | | | | | | | | | | | | |
| 7.5 | NA | NA | NA | NA | NA | NA | NA | NA | NA | NA | NA | 7.5 | 7.5 | |
| 5.0 | NA | NA | NA | NA | NA | NA | 5 | 5 | 5 | 5 | 5 | 5 | 5 | |
| 2.5 | NA | 2.5 | 2.5 | 2.5 | 2.5 | 2.5 | 2.5 | 2.5 | 2.5 | 2.5 | 2.5 | 2.5 | 2.5 | |
| Off OCS | 0 | 0 | 0 | 0 | 0 | 0 | 0 | 0 | 0 | 0 | 0 | 0 | 0 | |
| Therapy: 0 | | | | | | | | | | | | | | |

NA = not applicable;
OCS = oral corticosteroids (oral prednisone or equivalent)
^{a}Body weight should be rounded to the nearest whole number. For body weights that are not included in the table (<45 kg or >110 kg), the OCS equivalent doses can be based on the indicated daily OCS dose per kg of body weight. The proposed doses should be confirmed by the medical monitor.
^{b}The investigator should not deviate from the recommended concurrent oral prednisone dosing regimen, except when the safety of the participant may be compromised.
^{c}The duration of the tapering steps from escalated OCS doses (>0.5 mg/kg/day) may be shortened based on clinical judgment.

### Prohibited Medications

The following medications or treatments are not permitted while the participant receives IMP:
Any systemic treatment for BP except OCS, including but not limited to immunosuppressants, IVIg, SCIg, immunoadsorption, anti-CD20 biologics, tetracyclines with or without nicotinamide, dapsone, and methotrexate;
Topical corticosteroids (use must be discontinued before baseline);
Any other biologic agent or experimental/study IMP;
Live or live-attenuated vaccines;
Complementary therapies, including traditional Chinese medicines, herbs, or procedures (e.g., acupuncture), as assessed by the investigator.

As discussed below, some of these treatments can be administered as rescue therapy if the participant discontinues IMP.

### Rescue Therapy

Participants will discontinue IMP and start rescue therapy if any of the following occur:
The participant fails treatment with IMP (“treatment failure” as defined above);
The participant relapses twice;
The participant presents with steroid toxicity (or any severe AE deemed by the investigator to be related to OCS therapy).

Participants reporting any of the events mentioned above will discontinue study intervention and complete the assessments listed for the ETD visit (Table S4). These participants will continue attending study visits through the rest of the 36-week treatment period while receiving rescue therapy for BP (defined below). At week 36, these participants will complete the EoTP visit (Table S4); if these participants meet rollover eligibility requirements, they will be invited to immediately enroll in the OLE study ARGX-113-2010. Participants who do not meet rollover eligibility requirements or who choose not to enroll in the OLE study will participate in the 7-week treatment-free follow-up period.

Rescue therapy may include OCS, topical corticosteroids, immunosuppressants, tetracyclines with or without nicotinamide, dapsone, or methotrexate. As described above, these treatments are permitted as rescue therapy following discontinuation of IMP.

### D. Study Assessments and Procedures

Each participant should attend each study visit on the designated days (see Table S4 below, which presents the timing of visit activities to be performed throughout the entire study). There is a permissible visit window of ±2 days during the treatment period and ±3 days during the treatment-free follow-up period.

TABLE S4
Schedule of activities

| | Study Period | | | | | | | | | | | | | | |
|---|---|---|---|---|---|---|---|---|---|---|---|---|---|---|---|
| | Screening Period^{a} | Treatment Period^{b} | | | | | | | | | | | | | |
| | Visit Type | | | | | | | | | | | | | | |
| | SCR | BL | Mandatory On-site Visits | | | | | | | | | | | | |
| | Visit Week | | | | | | | | | | | | | | |
| | | 0 | 1 | (D 10) | 2 | 3 | 4 | 8 | 12 | 16 | 20 | 24 | 26 | 28 | 32 |
| | Visit Day | | | | | | | | | | | | | | |
| | −14 to −1 | 1 | 8 | 10 | 15 | 22 | 29 | 57 | 85 | 113 | 141 | 169 | 183 | 197 | 225 |
| | Visit Window (±days) | | | | | | | | | | | | | | |
| | +7 | — | ±2 | ±1 | ±2 | ±2 | ±2 | ±2 | ±2 | ±2 | ±2 | ±2 | ±2 | ±2 | ±2 |
| Informed consent^{g} | X | | | | | | | | | | | | | | |
| Inclusion/ | X | X | | | | | | | | | | | | | |
| exclusion criteria | | | | | | | | | | | | | | | |
| Randomization | | X | | | | | | | | | | | | | |
| Demography | X | | | | | | | | | | | | | | |
| Medical and surgical | X | | | | | | | | | | | | | | |
| history | | | | | | | | | | | | | | | |
| Histopathology/DIF^{h} | X | | | | | | | | | | | | | | |
| Karnofsky | X | | | | | | | | | | | | | | |
| performance index | | | | | | | | | | | | | | | |
| score | | | | | | | | | | | | | | | |
| QoL questionnaires | | X | | | | | | | X | | | | X | | |
| (EQ-5D-5L, DLQI, | | | | | | | | | | | | | | | |
| ABQoL) | | | | | | | | | | | | | | | |
| Concomitant | X | X | X | X | X | X | X | X | X | X | X | X | X | X | X |
| therapies/proceduresⁱ | | | | | | | | | | | | | | | |
| Height | X | | | | | | | | | | | | | | |
| Weight | X | X | X | | X | X | X | X | X | X | X | X | X | X | X |
| Physical examination | X | X | X | | X | X | X | X | X | X | X | X | X | X | X |
| and vital signs (blood | | | | | | | | | | | | | | | |
| pressure, HR, body | | | | | | | | | | | | | | | |
| temperature) | | | | | | | | | | | | | | | |
| ECG | X | | | | | | | | | | | | X | | |
| Urinalysis | X | X | | | | | X | X | X | X | X | X | | X | X |
| Active viral infection | X | | | | | | | | | | | | | | |
| tests (HBV, HCV, | | | | | | | | | | | | | | | |
| HIV) | | | | | | | | | | | | | | | |
| COVID-19 test | X | | | | | | | | | | | | | | |
| (performed according | | | | | | | | | | | | | | | |
| to local | | | | | | | | | | | | | | | |
| requirements) | | | | | | | | | | | | | | | |
| QuantiFERON | X | | | | | | | | | | | | | | |
| TB-test | | | | | | | | | | | | | | | |
| Serum pregnancy or | | | | | | | | | | | | | | | |
| serum FSH testⁱ | | | | | | | | | | | | | | | |
| Urine pregnancy test | | X | | | | | X | X | X | X | X | X | | X | X |
| (local laboratory)^{k} | | | | | | | | | | | | | | | |
| Clinical chemistry | X^{l} | X | X | | X | | X | X | X | X | X | X | X | X | X |
| and hematology | | | | | | | | | | | | | | | |
| Pharmacokinetics | | X | X | X^{m} | X | | X | X | X | X | X | X | X | X | X |
| Immunogenicity | X | X | | | X | | X | X | X | X | X | X | X | X | X |
| Pharmacodynamics | X | X | X | | X | | X | X | X | X | X | X | X | X | X |
| (total IgG serum | | | | | | | | | | | | | | | |
| levels; anti-BP180 | | | | | | | | | | | | | | | |
| and anti-BP230 | | | | | | | | | | | | | | | |
| antibodies; IgE | | | | | | | | | | | | | | | |
| serum levels) | | | | | | | | | | | | | | | |
| Substudy (selected | | X | | | | | X | | | | | | | | |
| sites): historical | | | | | | | | | | | | | | | |
| protective antibody | | | | | | | | | | | | | | | |
| titers (serum) | | | | | | | | | | | | | | | |
| Substudy (selected | | Before vaccination; first available on- | | | | | | | | | | | | | |
| sites): vaccination | | site visits at +4 weeks, +12 | | | | | | | | | | | | | |
| antibody titers | | weeks, and +24 weeks postvaccination | | | | | | | | | | | | | |
| (serum) | | | | | | | | | | | | | | | |
| Substudy (selected | | Before vaccination; first available | | | | | | | | | | | | | |
| sites): vaccination | | on-site visits at +12 weeks | | | | | | | | | | | | | |
| cellular responses | | and +24 weeks postvaccination | | | | | | | | | | | | | |
| (PBMCs) | | | | | | | | | | | | | | | |
| IGA-BP | X | X | X | | X | X | X | X | X | X | X | X | X | X | X |
| BPDAI | X | X | X | | X | X | X | X | X | X | X | X | X | X | X |
| Itch NRS | | X | X | | X | X | X | X | X | X | X | X | X | X | X |
| BP disease statusⁿ | | X | X | | X | X | X | X | X | X | X | X | X | X | X |
| Concurrent OCS | | X | X | | X | X | X | X | X | X | X | X | X | X | X |
| dose monitoring^{o} | | | | | | | | | | | | | | | |
| Investigator | | | | | | | | | | | | | | | |
| telephone calls^{p} | | | | | | | | | | | | | | | |
| Substudy (selected | | The sponsor suggests that photos be taken | | | | | | | | | | | | | |
| sites): photography | | at BL and when CDA, CR, and relapse occur. | | | | | | | | | | | | | |
| of BP lesions | | Photos may also be taken at intermediate | | | | | | | | | | | | | |
| | | timepoints per investigator discretion. | | | | | | | | | | | | | |
| GTI (including | | X | | | | | | | X | | | | X | | |
| C-GTI & GTI-SL) | | | | | | | | | | | | | | | |
| AEs | X | X | X | X | X | X | X | X | X | X | X | X | X | X | X |
| IMP administration^{q} | | X | X | | X | X | X | X | X | X | X | X | X | X | X |
| IMP (self-) | | | | | X | X | X | X | X | X | X | X | X | X | X |
| administration | | | | | | | | | | | | | | | |
| training | | | | | | | | | | | | | | | |
| Rescue therapy | | | | | | | | | | | | | | | |
| evaluation/initiation | | | | | | | | | | | | | | | |

| | Study Period | | | | | | |
|---|---|---|---|---|---|---|---|
| | | | Follow-up | | | | |
| | Treatment Period^{b} | | Period^{c} | | Other Visits | | |
| | Visit Type | | | | | | |
| | Other | | | | | | |
| | Weekly | | | | | | |
| | Visits | EoTP | F1 | F2 | ETD^{d} | ESD^{e} | UNS^{f} |
| | Visit Week | | | | | | |
| | — | 36 | 39 | 43 | ETD | ESD | |
| | Visit Day | | | | | | |
| | | 253 | | | | | |
| | Visit Window (±days) | | | | | | |
| | ±2 | ±2 | ±3 | ±3 | — | — | — |
| Informed consent^{g} | | | | | | | |
| Inclusion/ | | | | | | | |
| exclusion criteria | | | | | | | |
| Randomization | | | | | | | |
| Demography | | | | | | | |
| Medical and surgical | | | | | | | |
| history | | | | | | | |
| Histopathology/DIF^{h} | | | | | | | |
| Karnofsky | | | | | | | |
| performance index | | | | | | | |
| score | | | | | | | |
| QoL questionnaires | | X | | | X | X | |
| (EQ-5D-5L, DLQI, | | | | | | | |
| ABQoL) | | | | | | | |
| Concomitant | X | X | X | X | X | X | X |
| therapies/proceduresⁱ | | | | | | | |
| Height | | | | | | | |
| Weight | | X | X | X | X | X | |
| Physical examination | | X | X | X | X | X | X |
| and vital signs (blood | | | | | | | |
| pressure, HR, body | | | | | | | |
| temperature) | | | | | | | |
| ECG | | X | | | X | X | |
| Urinalysis | | X | X | X | X | X | X |
| Active viral infection | | | | | | | |
| tests (HBV, HCV, | | | | | | | |
| HIV) | | | | | | | |
| COVID-19 test | | | | | | | |
| (performed according | | | | | | | |
| to local | | | | | | | |
| requirements) | | | | | | | |
| QuantiFERON | | | | | | | |
| TB-test | | | | | | | |
| Serum pregnancy or | | | | | | | |
| serum FSH testⁱ | | | | | | | |
| Urine pregnancy test | | X | X | X | X | X | X |
| (local laboratory)^{k} | | | | | | | |
| Clinical chemistry | | X | X | X | X | X | X |
| and hematology | | | | | | | |
| Pharmacokinetics | | X | X | X | X | X | X |
| Immunogenicity | | X | X | X | X | X | X |
| Pharmacodynamics | | X | X | X | X | X | X |
| (total IgG serum | | | | | | | |
| levels; anti-BP180 | | | | | | | |
| and anti-BP230 | | | | | | | |
| antibodies; IgE | | | | | | | |
| serum levels) | | | | | | | |
| Substudy (selected | | | | | | | |
| sites): historical | | | | | | | |
| protective antibody | | | | | | | |
| titers (serum) | | | | | | | |
| Substudy (selected | Before vaccination; first available on- | | | (x) | | (x) | |
| sites): vaccination | site visits at +4 weeks, +12 | | | | | | |
| antibody titers | weeks, and +24 weeks postvaccination | | | | | | |
| (serum) | | | | | | | |
| Substudy (selected | Before vaccination; first available | | | (x) | | (x) | |
| sites): vaccination | on-site visits at +12 weeks | | | | | | |
| cellular responses | and +24 weeks postvaccination | | | | | | |
| (PBMCs) | | | | | | | |
| IGA-BP | | X | X | X | X | X | X |
| BPDAI | | X | X | X | X | X | X |
| Itch NRS | | X | X | X | X | X | X |
| BP disease statusⁿ | X | X | X | X | X | X | X |
| Concurrent OCS | X | X | X | X | X | X | X |
| dose monitoring^{o} | | | | | | | |
| Investigator | X^{p} | | | | | | |
| telephone calls^{p} | | | | | | | |
| Substudy (selected | | The sponsor suggests that photos | | | | | |
| sites): photography | | be taken at BL and when CDA, CR, | | | | | |
| of BP lesions | | and relapse occur. Photos may also | | | | | |
| | | be taken at intermediate timepoints | | | | | |
| | | per investigator discretion. | | | | | |
| GTI (including | | X | | | X | X | |
| C-GTI & GTI-SL) | | | | | | | |
| AEs | X | X | X | X | X | X | X |
| IMP administration^{q} | X | | | | | | |
| IMP (self-) | | | | | | | |
| administration | | | | | | | |
| training | | | | | | | |
| Rescue therapy | | | | | X | | |
| evaluation/initiation | | | | | | | |

ABQoL = Autoimmune Bullous Disease Quality of Life questionnaire; AE = adverse event; aPTT = activated partial thromboplastin time; BL = baseline; BP = bullous pemphigoid; BPDAI = Bullous Pemphigoid Disease Area Index; CDA = control of disease activity; C-GTI = composite Glucocorticoid Toxicity Index; DIF = direct immunofluorescence; DLQI = Dermatology Life Quality Index; ECG = electrocardiogram; EFG = efgartigimod; EFG PH20 SC = efgartigimod coformulated with rHuPH20 for subcutaneous injection (dosing formulation); EoTP = end of treatment period; EQ-5D-5L = EuroQoL 5-Dimension 5-Level; ESD = early study discontinuation; ETD = early treatment discontinuation; FSH = follicle-stimulating hormone; F1 and F2 = follow-up visits 1 and 2; GTI = Glucocorticoid Toxicity Index; GTI-SL = Glucocorticoid Toxicity Index Specific List; HBV = hepatitis B virus; HCV = hepatitis C virus; HR = heart rate; IGA-BP = Investigator Global Assessment of Bullous Pemphigoid; IgE = immunoglobulin type-E; IgG = immunoglobulin type-G; IMP = investigational medicinal product; INR = international normalized ratio; Itch NRS = Itch Numerical Rating Scale; OCS = oral corticosteroid(s); OLE = open-label extension (study); PBMCs = peripheral blood mononuclear cells; PBO PH20 SC = placebo coformulated with rHuPH20 for subcutaneous injection (dosing formulation); PK = pharmacokinetic; QoL = quality of life; rHuPh20 = recombinant human hyaluronidase PH20; SCR = screening; TB = tuberculosis; UNS = unscheduled; WOCBP = women of childbearing potential.
^{a}The screening period may be extended by a maximum of 7 days for logistical or administrative reasons (e.g., receipt of laboratory results).
^{b}The treatment period consists of weekly visits, starting with the BL visit and ending with the EoTP visit. Mandatory on-site visits must be performed as indicated. Weekly visits that are not designated as mandatory on-site visits may be performed at home if the participant has achieved CDA. During home visits, IMP will be administered by a home nurse.
^{c}Follow-up visits are only applicable for participants who choose not to roll over to the OLE study ARGX-113-2010.
^{d}The ETD visit must be performed when IMP is discontinued before the EoTP visit; the study site should make every effort to perform this visit within 7 days of the participant's last IMP dose. Participants will remain in the study (provided that they have not withdrawn consent) and will continue to participate in study visits as scheduled, only without IMP administration. Rescue therapy may be initiated at this visit.
^{e}The ESD visit must be performed when the participant either withdraws consent or is permanently discontinued from the study. The study site must make every effort to perform this visit within 7 days of the participant's last IMP dose.
^{f}An unscheduled visit should be performed if the participant has (or suspects they have) new BP lesions, has new AEs, or has other issues requiring site staff intervention.
^{g}No study-related activities can be performed before the participant has provided signed informed consent.
^{h}Histopathology/DIF are performed only if positive test results are not available as part of the participant's medical history.
ⁱAll vaccinations received prior to the study should be recorded as part of the participant's prior medication history, while all vaccinations received during the study should be recorded as concomitant medications.
^{j}WOCBP must have a negative serum pregnancy test and must be performed in WOCBP. Alternatively, postmenopausal female participants must have a serum FSH test to confirm postmenopausal status.
^{k}Urine pregnancy tests will be performed at the specified visits for WOCBP only.
^{l}In addition to the standard clinical chemistry and hematology parameters, the screening assessment also includes INR and aPTT.
^{m}The day 10 PK blood sample will only be collected during part A of the study. The sample should be collected no earlier than day 9, and no later than day 11.
ⁿBP disease assessment must be performed according to the definitions presented herein.
^{o}The participant's concurrent prednisone (or alternate OCS of equivalent dose strength) dosage will be monitored throughout the study and will be adjusted based on the procedures described herein.
^{p}For study visits that occur at home (in between mandatory on-site visits), the investigator will call the participant once weekly to assess BP disease status and review/adjust the participant's concurrent prednisone dosage.
^{q}At BL (week 0 [day 1]) and week 1 (day 8), 2000 mg of IMP (EFG PH20 SC or PBO PH20 SC) will be administered via 2 SC injections. At weeks 2 through 35, 1000 mg of IMP (EFG PH20 SC or PBO PH20 SC) will be administered via a single SC injection. To monitor for possible injection-related reactions, the participant will remain at the study site for at least 1 hour after receiving their first dose of IMP at BL (week 0 [day 1]), at least 30 minutes after receiving their IMP doses at weeks 1-4, and at least 15 minutes after receiving their IMP doses at subsequent visits. Following completion of these monitoring periods, participants will be released after site staff confirm stable clinical status.

### Assessment of BP Disease Status and OCS Dose Monitoring

The efficacy of treatment will be assessed at on-site visits by the investigator, who will assess the BP disease status (CDA, PR, CR, relapse, treatment failure) of the participant according to the definitions/criteria presented herein. The investigator will also record the daily OCS dose administered to the participant since the last visit, adjust the daily OCS dose (taper, stop, reinitiate, or increase) based on the participant's BP disease status, and record treatment failures during on-site visits. For home visits occurring between mandatory on site visits, the investigator will call the participant once weekly to review the current BP disease status and OCS dosing regimen.

### IGA-BP

TBP disease activity and severity will be assessed by the investigator using the IGA-BP. The IGA-BP is a new assessment tool that was developed in 2021 by medical experts in the field of AIBDs, and it is currently being used (along with the BPDAI) in other clinical studies of therapies for BP. The IGA categorizes the severity of BP on a numerical scale of 0 (clear) to 4 (severe).

The IGA-BP will be administered during all on site visits (Table S4).

### BPDAI

In addition to the IGA-BP, BP disease activity will also be assessed by the investigator using the BPDAI. The BPDAI is an internationally validated tool to objectively measure disease activity. The BPDAI differentiates scores for skin (erosions/blisters and urticaria/erythema) and mucous membrane activity in several anatomical locations. In addition, separate scores for damage (e.g., pigmentation) are recorded to account for healing lesions. The BPDAI will be administered during all on site visits (Table S4).

### Itch-NRS

Pruritic symptoms of BP will be indicated by the participant on the Itch NRS, recording an average and a worst score for itch suffered within the past 24 hours.

The Itch NRS will be administered during all mandatory on-site visits beginning at baseline (week 0) (Table S4).

### Quality of Life (QoL) Assessments

Participants will complete the following PRO assessments of their QOL at the timepoints listed in the SoA (Table S4):
EuroQol 5-dimension 5-level Scale (EQ-5D 5L): the EQ-5D 5L is a standardized measure of health status developed by the EuroQOL Group to provide a simple, generic measure of health for clinical and economic appraisal. The descriptive system comprises 5 dimensions (mobility, self-care, usual activities, pain/discomfort, anxiety/depression). Each dimension now has 5 levels: no problems, slight problems, moderate problems, severe problems, and extreme problems.
Dermatology Life Quality Index (DLQI): the DLQI consists of 10 questions concerning the participant's perception of the impact of skin diseases on different aspects of their health-related QOL the previous week. The impact of each aspect on the QoL assessment is scored qualitatively, ranging from “not at all” to “very much.”
Autoimmune Bullous Disease Quality of Life Index (ABQOL) questionnaire: the ABQOL was developed and validated for determining the impact of AIBDs and their therapies on the daily life of patients. It is a series of 17 questions concerning the participant's perceptions of how AIBD is affecting their daily lives, including comfort, hygiene, eating/drinking, appearance, social interactions, sexual activity, and employment.

### Glucocorticoid Toxicity Index

As an additional assessment of the impact of glucocorticoid morbidity, the GTI v2.0 will be assessed at the visits listed in the SoA (Table S4). The GTI v2.0 is a complementary scoring system to the overall report of AEs that are judged as related to glucocorticoids by investigators during interventional studies. It also enables the monitoring of long-term tolerability of glucocorticoids during their prolonged use during clinical practice.

The GTI v2.0 consists of the following 2 instruments:
The Composite-GTI (C-GTI), which serves as a primary instrument intended to capture toxicities that are likely due to glucocorticoid exposure. The C-GTI has 9 functional domains: BMI, glucose control, blood pressure, lipid metabolism, bone mineral density, muscle strength, skin toxicity, neuropsychiatric effects, and infection. Each domain includes several weighted items that correspond to varying degrees of glucocorticoid toxicity. Note: the bone mineral density domain will not be used in this study, because it is typically excluded from studies <1 year in duration, due to the fact that bone densitometry is not sufficiently reliable in measuring changes over shorter durations. Two analytical scores are generated from the weighted C-GTI items: the Cumulative Worsening Score (CWS) and the AIS. The CWS is designed to assess cumulative glucocorticoid toxicity, while the AIS can be used to assess whether a new therapy is effective in reducing glucocorticoid toxicity over time. Together, the CWS and AIS provide complementary information about the ability of an investigational agent to reduce overall glucocorticoid toxicity.
The Specific List of the GTI (GTI-SL) captures well-known glucocorticoid-related side effects. This non-weighted instrument provides additional information for the domains most affected by glucocorticoid use during the participant's course of treatment. It comprises 11 domains (9 of which are shared in common with the C-GTI) and 23 items. Note: for the reason mentioned above, the GTI SL item covering bone mineral density decrease (part of the Bone Health domain) will not be used in this study.

The tabular components of the modified GTI as adapted for this study are presented in Tables S5-S6.

TABLE S5
Modified GTI 2.0 composite index

| Domain | | Score |
|---|---|---|
| 1. Change in Body | Decrease by ≥5 BMI units | −36 |
| Weight (BMI) | Decrease by >2 but <5 BMI units | −21 |
| | No significant change (±2 BMI units) | 0 |
| | Increase of >2 to <5 BMI units | 21 |
| | Increase of ≥5 BMI units | 36 |
| 2. Glucose Metabolism | Improvement in HbA1c AND decrease in medication | −44 |
| | Improvement in HbA1c OR decrease in medication | −32 |
| | No significant change | 0 |
| | Increase in HbA1c OR increase in medication | 32 |
| | Increase in HbA1c AND increase in medication | 44 |
| 3. Blood Pressure | Improvement in blood pressure AND decrease in medication | −44 |
| | Improvement in blood pressure OR decrease in medication | −19 |
| | No significant change | 0 |
| | Increase in blood pressure OR increase in medication | 19 |
| | Increase in blood pressure AND increase in medication | 44 |
| 4. Hyperlipidemia | Decrease in LDL AND decrease in medication | −30 |
| | Decrease in LDL OR decrease in medication | −10 |
| | No significant change | 0 |
| | Increase in LDL OR increase in medication | 10 |
| | Increase in LDL AND increase in medication | 30 |
| 5. Corticosteroid | Moderate weakness to none | −63 |
| Myopathy | Moderate-to-mild weakness | −54 |
| | Mild weakness to none | −9 |
| | No significant change | 0 |
| | None-to-mild weakness (without functional limitation) | 9 |
| | Mild-to-moderate weakness | 54 |
| | None-to-moderate weakness (with functional limitation) | 63 |
| 6. Skin Corticosteroid- | Decrease in skin toxicity-moderate to none | −26 |
| Related Toxicity | Decrease in skin toxicity-moderate to mild | −18 |
| | Decrease in skin toxicity-mild to none | −8 |
| | No significant change | 0 |
| | Increase in skin toxicity-none to mild | 8 |
| | Increase in skin toxicity-mild to moderate | 18 |
| | Increase in skin toxicity-none to moderate | 26 |
| 7. Neuropsychiatric- | Decrease in NP toxicity-moderate to none | −74 |
| Corticosteroid-Related | Decrease in NP toxicity-moderate to mild | −63 |
| Symptoms | Decrease in NP toxicity-mild to none | −11 |
| | No significant change | 0 |
| | Increase in NP toxicity-none to mild | 11 |
| | Increase in NP toxicity-mild to moderate | 63 |
| | Increase in NP toxicity-none to moderate | 74 |
| 8. Infection | No infection | 0 |
| | Oral or vaginal candidiasis or uncomplicated zoster (<grade 3) | 19 |
| | Grade 3, 4, or 5 infection | 93 |

Source: Adapted from McDowell et al., J Allergy Clin Immunol. 2021; 9(1): 365-372.
BMI = body mass index;
GTI = Glucocorticoid Toxicity Index;
HbA1c = glycated hemoglobin;
LDL = low-density lipoprotein;
NP = neuropsychiatric

TABLE S6
Modified GTI 2.0 specific list

| | | |
|---|---|---|
| | At | New |
| Domain | Baseline | Since |
| Item | or Before | Baseline |

BMI
An absolute increase in BMI of more than 8 units (and >24.9 kg/m²)
Blood pressure
Hypertensive emergency (see definition)
Posterior reversible encephalopathy syndrome (see definition)
Endocrine
Symptomatic adrenal insufficiency
Bone health^{a}
Osteonecrosis of 1 joint
Osteonecrosis of >1 joint
Insufficiency fracture
Insufficiency fracture in more than 1 bone
Muscle and tendon
Severe glucocorticoid myopathy (see definition)
Tendon rupture
More than 1 tendon rupture
Eye
Central serous retinopathy
New-onset or worsened elevation of intraocular pressure requiring treatment or
change in treatment
Posterior subcapsular cataracts (or history of same)
Infection
Grade 4 infection (see definition)
Grade 5 infection (death from infection)
Glucose tolerance
Diabetic nephropathy
Diabetic neuropathy
Diabetic retinopathy
Gastrointestinal tract
Gastrointestinal perforation (occurring in the absence of regular nonsteroidal
anti-inflammatory drug use)
Peptic ulcer disease confirmed by endoscopy (excluding Helicobacter pylori)
Skin
Severe skin toxicity (see definition)
Neuropsychiatric
Psychosis, defined as hallucinations, delusions, or disorganized thought processes
(occurring in the absence of mania, delirium, or depression)
Glucocorticoid-induced violence toward self or others
Other glucocorticoid toxicities
Please specify: _{——}_{——}_{——}
Source: Adapted from McDowell et al., J Allergy Clin Immunol. 2021; 9(1): 365-372.
BMI—body mass index;
GTI = Glucocorticoid Toxicity Index
^{a}For the reasons described above, bone mineral density will not be assessed in ARGX-113-2009. Therefore, the bone health parameter “bone mineral density decrease >6%” has been removed from this list.

### E. Pharmacokinetics

Blood samples for PK analysis will be collected from each participant as described in the SoA (Table S4). Samples will be collected pre-dose on IMP administration visits (within ≤2 hours before IMP administration). During unscheduled visits, blood samples for PK will be collected only if study intervention is administered.

In part A only, 1 additional PK sample will be collected from all participants on day 10 (±1 day; should be collected at least 2 days after the second IMP dose is administered at week 1).

Efgartigimod serum concentrations will be determined using a validated assay.

The actual date and time of collection of each blood sample will be recorded in the requisition form. If no sample is taken, the reason will be recorded in the relevant section of the eCRF.

### F. Pharmacodynamics

Blood samples will be collected for the determination of PD markers (total IgG serum levels, anti-BP180 antibodies, and anti-BP230 antibodies, IgE serum levels) as described in the SoA (Table S4).

For baseline and all postbaseline PD assessment timepoints (Table S4), sample collection will be performed pre-dose at IMP administration visits (within ≤2 hours before IMP administration). PD markers will be determined using validated assays.

The actual date and time of collection of these blood samples will be recorded in the requisition form. If no sample is taken, the reason will be recorded in the relevant section of the eCRF.

PD blood samples that are collected at the screening visit may be used for methodology validation and/or for future research purposes. Such use of these samples is only permitted after obtaining consent from the participant.

### G. Immunogenicity Assessments

Blood samples will be collected to assess the serum levels of antidrug antibodies (ADA) to efgartigimod and plasma levels of antibodies to rHuPH20 from all participants as indicated in the SoA (Table S4). Samples will be collected pre-dose on IMP administration visits (within ≤2 hours before IMP administration).

All samples will be analyzed in a 3-tiered approach using validated immunogenicity methods. First, all samples will be evaluated in a screening assay (tier 1) and scored as positive or negative. Secondly, screened positive samples will be evaluated in a confirmatory assay to assess the specificity of the immunogenicity response. The samples will be scored as confirmed positive or confirmed negative. Samples confirmed positive in tier 2 will be further analyzed in a titration assay to characterize the magnitude of the antibody response and a neutralizing antibody (Nab) assay to assess the antibodies for neutralizing activity.

The actual date and time of collection of these blood samples will be recorded in the requisition form. If no sample is taken, the reason will be recorded in the relevant section of the eCRF.

Immunogenicity blood samples that are collected at the screening visit may be used for methodology validation and/or for future research purposes. Such use of these samples is only permitted after obtaining consent from the participant.

### H. Objectives and Endpoints

Study objectives and related endpoints are described in the tables below for part A (Table S7) and part B (Table S8) of the study.

TABLE S7
Part A objectives and endpoints

| | |
|---|---|
| Objectives | Endpoints |

Primary

| | |
|---|---|
| To evaluate the efficacy | Proportion of participants in CR while receiving |
| of efgartigimod PH20 SC on achieving | efgartigimod PH20 SC or placebo and off OCS therapy |
| sustained remission in the treatment | for ≥8 weeks at week 36 |
| of participants with BP | |

Secondary

| | |
|---|---|
| To evaluate the corticosteroid-sparing | Cumulative dose of OCS from baseline to week 36 |
| effects of efgartigimod PH20 SC in | |
| participants with BP | |
| To characterize the overall efficacy | Proportion of participants with an IGA-BP score of 0 |
| of efgartigimod PH20 SC in the | while receiving efgartigimod PH20 SC or placebo and |
| treatment of participants with BP | off OCS therapy for ≥8 weeks at week 36 |
| | Proportion of participants with an (IGA-BP score of 0 |
| | or 1 while receiving efgartigimod PH20 SC or placebo |
| | and off OCS therapy for ≥8 weeks at week 36 |
| | Proportion of participants who achieve an IGA-BP |
| | score of 0 or 1 while receiving efgartigimod PH20 SC |
| | or placebo at any time through week 36 |
| | Changes from baseline in the BPDAI activity score |
| | Proportion of participants who are in CR while |
| | receiving efgartigimod PH20 SC or placebo and |
| | receiving minimal OCS therapy for ≥8 weeks |
| | at week 36. Minimal OCS therapy is defined as ≤0.1 |
| | mg/kg/day of prednisone (or equivalent). |
| | Time to achieve the following: |
| | CDA |
| | CR |
| | CR while on minimal OCS therapy for ≥8 weeks |
| | CR/PR while off OCS therapy for ≥8 weeks |
| | CR while off OCS therapy for ≥8 weeks |
| | Relapse |
| To characterize the overall efficacy | Cumulative OCS dose for the participant at the |
| of efgartigimod PH20 SC in the | timepoints when they exhibit the following: |
| treatment of participants with BP | CDA |
| (continued) | CR |
| | CR while on minimal OCS therapy for ≥8 weeks |
| | CR/PR while off OCS therapy for ≥8 weeks |
| | CR while off OCS therapy for ≥8 weeks |
| | Relapse |
| | Proportion of participants receiving rescue therapy |
| | before week 36 |
| To evaluate the efficacy of | Proportion of participants who achieve CDA while |
| efgartigimod PH20 SC in preventing | receiving efgartigimod PH20 SC or placebo and remain |
| relapse of BP | free of relapse through week 36 |
| To evaluate the effect of | Changes from baseline in the 24-hour average itch score |
| efgartigimod PH20 SC on pruritus in | from the Itch NRS |
| participants with BP | Changes from baseline in the 24-hour worst itch score |
| | from the Itch NRS |
| To assess the safety and tolerability of | Incidence and severity of TEAEs, AESIs, and SAEs |
| efgartigimod PH20 SC administered to | Vital sign measurements, physical examinations, ECGs, |
| participants with BP | and clinical laboratory safety evaluations |
| To assess glucocorticoid-associated | The Aggregate Improvement Score (AIS) from the GTI |
| morbidity and evaluate the impact of | The Cumulative Worsening Score (CWS) from the GTI |
| efgartigimod PH20 SC in reduction of | The Glucocorticoid Toxicity Index Specific List |
| glucocorticoid toxicity | (GTI-SL) |
| To evaluate the effects of | EuroQol 5-Dimension 5-Level (EQ-5D-5L) scores over |
| efgartigimod PH20 SC on the QoL of | time |
| participants with BP | Dermatology Life Quality Index (DLQI) scores over |
| | time |
| | Autoimmune Bullous Disease Quality of Life (ABQoL) |
| | scores over time |
| To evaluate the pharmacokinetics | EFG serum concentrations |
| of efgartigimod PH20 SC in participants | |
| with BP | |
| To evaluate the pharmacodynamics | Total IgG serum levels |
| of efgartigimod PH20 SC in participants | Anti-BP180 and anti-BP230 antibodies |
| with BP | |
| To evaluate the immunogenicity of | Antidrug antibodies (ADA) to efgartigimod (in serum) |
| efgartigimod PH20 SC in participants | and antibodies produced against rHuPH20 (in plasma) |
| with BP | |
| To evaluate the competency | Number and percentage of participants (or their |
| of participants or caregivers | caregivers) who complete the (self-)administration |
| to (self-)administer | training at study sites |
| efgartigimod PH20 SC | Number and percentage of participants (or their |
| | caregivers) who are determined by site staff to be |
| | sufficiently competent in (self-)administering |
| | efgartigimod PH20 SC |
| | Number and percentage of participants (or their |
| | caregivers) who successfully (self-)administer |
| | efgartigimod PH20 SC under site staff supervision |

Exploratory

| | |
|---|---|
| To further explore the | IgE serum levels |
| pharmacodynamics of | |
| efgartigimod PH20 SC in | |
| participants with BP | |

ABQoL = Autoimmune Disease Quality of Life (questionnaire);
AE = adverse event;
AESI = adverse event of special interest;
BP = bullous pemphigoid;
BPDAI = Bullous Pemphigoid Disease Area Index;
CDA = control of disease activity;
CR = complete remission;
DLQI = Dermatology Life Quality Index;
ECG = electrocardiogram;
EFG = efgartigimod;
efgartigimod PH20 SC = efgartigimod with rHuPH20 (coformulation) for SC injection;
EQ-5D-5L = EuroQoL 5-Dimension 5-Level;
IGA-BP = Investigator Global Assessment of Bullous Pemphigoid;
IgE = immunoglobulin type-E;
IgG = immunoglobulin type-G;
IMP = investigational medicinal product (efgartigimod PH20 SC or PBO PH20 SC);
NRS = numerical rating scale;
OCS = oral corticosteroid (oral prednisone or an alternate corticosteroid of equivalent dose strength);
PBO PH20 SC = placebo with rHuPH20 (coformulation) for SC injection;
PR = partial remission;
QoL = quality of life;
rHuPH20 = recombinant human hyaluronidase PH20;
SAE = serious adverse event;
TEAE = treatment-emergent adverse event

TABLE S8
Part B objectives and endpoints

| | |
|---|---|
| Objectives | Endpoints |

Primary

| | |
|---|---|
| To evaluate the efficacy | Proportion of participants who are in CR while receiving |
| of efgartigimod PH20 SC on achieving | efgartigimod PH20 SC or placebo and off OCS therapy |
| sustained remission in the treatment | for ≥8 weeks at week 36 |
| of participants with BP | |

Key Secondary - Subject to Alpha Control

| | |
|---|---|
| 1. To evaluate the corticosteroid-sparing | 1. Cumulative dose of OCS from baseline to week 36 |
| effects of efgartigimod PH20 SC in | |
| participants with BP | |
| 2. To characterize the overall efficacy | 2. Proportion of participants who achieve an IGA-BP |
| of efgartigimod PH20 SC in the | score of 0 while receiving efgartigimod PH20 SC or |
| treatment of participants with BP | placebo and have been off OCS therapy for ≥8 weeks at |
| | week 36 |
| 3. To evaluate the efficacy | 3. Proportion of participants who achieve control |
| of efgartigimod PH20 SC in preventing | of disease activity (CDA) while receiving efgartigimod |
| relapse of BP | PH20 SC or placebo and remain free of relapse through |
| | week 36 |
| 4. To characterize the efficacy | 4. Proportion of participants who are in complete |
| of efgartigimod PH20 SC in the | remission (CR) while receiving efgartigimod PH20 SC |
| treatment of participants with BP | or placebo and have been receiving minimal OCS |
| | therapy for ≥8 weeks at week 36. Minimal OCS |
| | therapy is defined as ≤0.1 mg/kg/day of prednisone (or |
| | an equivalent dose of another OCS) |
| 5. To evaluate the effect of | 5. Changes from baseline in the 24-hour average itch |
| efgartigimod PH20 SC on pruritus in | score from the Itch NRS |
| participants with BP | |

Other Secondary

| | |
|---|---|
| To further characterize the overall | Proportion of participants who achieve an IGA-BP |
| efficacy of efgartigimod PH20 SC in the | score of 0 or 1 while receiving efgartigimod PH20 SC |
| treatment of participants with BP | or placebo and have been off OCS therapy for ≥8 weeks |
| | at week 36 |
| | Proportion of participants who achieve an IGA-BP |
| | score of 0 or 1 while receiving efgartigimod PH20 SC |
| | or placebo at any time through week 36 |
| | Changes from baseline in the BPDAI activity score |
| | Time to achieve the following: |
| | CDA |
| | CR |
| | CR while on minimal OCS therapy for ≥8 weeks |
| | CR/PR while off OCS therapy for ≥8 weeks |
| | CR while off OCS therapy for ≥8 weeks |
| | Relapse |
| | Cumulative OCS dose for the participant at the |
| | timepoints where they exhibit the following: |
| | CDA |
| | CR |
| | CR while on minimal OCS therapy for ≥8 weeks 0 |
| | CR/PR while off OCS therapy for ≥8 weeks |
| | CR while off OCS therapy for ≥8 weeks |
| | Relapse |
| | Changes from baseline in the 24-hour worst itch score |
| | from the Itch NRS |
| | Proportion of participants who receive rescue therapy |
| | before week 36 |
| To assess the safety and tolerability of | Incidence and severity of TEAEs, AESIs, and SAEs |
| efgartigimod PH20 SC administered to | Vital sign measurements, physical examinations, ECGs, |
| participants with BP | and clinical laboratory safety evaluations |
| To assess glucocorticoid-associated | The Aggregate Improvement Score (AIS) from the |
| morbidity and evaluate the impact of | Glucocorticoid Toxicity Index (GTI) |
| efgartigimod PH20 SC in reduction of | The Cumulative Worsening Score (CWS) from the GTI |
| glucocorticoid toxicity | The Glucocorticoid Toxicity Index Specific List |
| | (GTI-SL) |
| To evaluate the effects of | EuroQol 5-Dimension 5-Level (EQ-5D-5L) scores over |
| efgartigimod PH20 SC on the QoL of | time |
| participants with BP | Dermatology Life Quality Index (DLQI) scores over |
| | time |
| | Autoimmune Bullous Disease Quality of Life (ABQOL) |
| | scores over time |
| To evaluate the pharmacokinetics | EFG serum concentrations |
| of efgartigimod PH20 SC in participants | |
| with BP | |
| To evaluate the pharmacodynamics | Total IgG serum levels |
| of efgartigimod PH20 SC in participants | Anti-BP180 and anti-BP230 antibodies |
| with BP | |
| To evaluate the immunogenicity of | Antidrug antibodies (ADA) to EFG (in serum) |
| efgartigimod PH20 SC in participants | and antibodies produced against rHuPH20 (in plasma) |
| with BP | |
| To evaluate the competency | Number and percentage of participants (or their |
| of participants or caregivers | caregivers) who complete the (self-)administration |
| to (self-)administer | training at study sites |
| efgartigimod PH20 SC | Number and percentage of participants (or their |
| | caregivers) who are determined by site staff to be |
| | sufficiently competent in (self-)administering |
| | efgartigimod PH20 SC |
| | Number and percentage of participants (or their |
| | caregivers) who successfully (self-)administer |
| | efgartigimod PH20 SC under site staff supervision |

Exploratory

| | |
|---|---|
| To further explore the | IgE serum levels |
| pharmacodynamics of | |
| efgartigimod PH20 SC in participants | |
| with BP | |

ABQoL = Autoimmune Disease Quality of Life (questionnaire);
AE = adverse event;
AESI = adverse event of special interest;
BP = bullous pemphigoid;
BPDAI = Bullous Pemphigoid Disease Area Index;
CDA = control of disease activity;
CR = complete remission;
DLQI = Dermatology Life Quality Index;
ECG = electrocardiogram;
EFG = efgartigimod;
efgartigimod PH20 SC = efgartigimod with rHuPH20 (coformulation) for SC injection;
EQ-5D-5L = EuroQOL 5-Dimension 5-Level;
IGA-BP = Investigator Global Assessment of Bullous Pemphigoid;
IgE = immunoglobulin type-E;
IgG = immunoglobulin type-G;
IMP = investigational medicinal product (efgartigimod PH20 SC or PBO PH20 SC); NRS=numerical rating scale ;
OCS = oral corticosteroid (oral prednisone or an alternate corticosteroid of equivalent dose strength);
PBO PH20 SC = placebo with rHuPH20 (coformulation) for SC injection;
PR = partial remission;
QoL = quality of life;
rHuPH20 = recombinant human hyaluronidase PH20;
SAE = serious adverse event;
TEAE = treatment-emergent adverse event

### Example 3: An Open-Label Extension Study of Efgartigimod PH20 SC in Adult Participants Who Completed ARGX-113-2009 (BALLAD+)

This open-label extension (OLE) study will assess whether long-term administration of efgartigimod PH20 SC is safe and effective in adult participants with BP.

### A. Study Design

### Overall Design

ARGX-113-2010 is a prospective, multicenter, OLE study to investigate the long-term safety, tolerability, efficacy, participant outcome measures (including QoL assessments), PK/PD, and immunogenicity of efgartigimod PH20 SC in adult participants with BP who completed the antecedent study described in Example 2 (ARGX-113-2009; i.e., those who completed the end of treatment period (EoTP) visit at week 36 and then chose to enroll in this study). The duration of this OLE study will be 52 weeks.

### Treatment Regimens

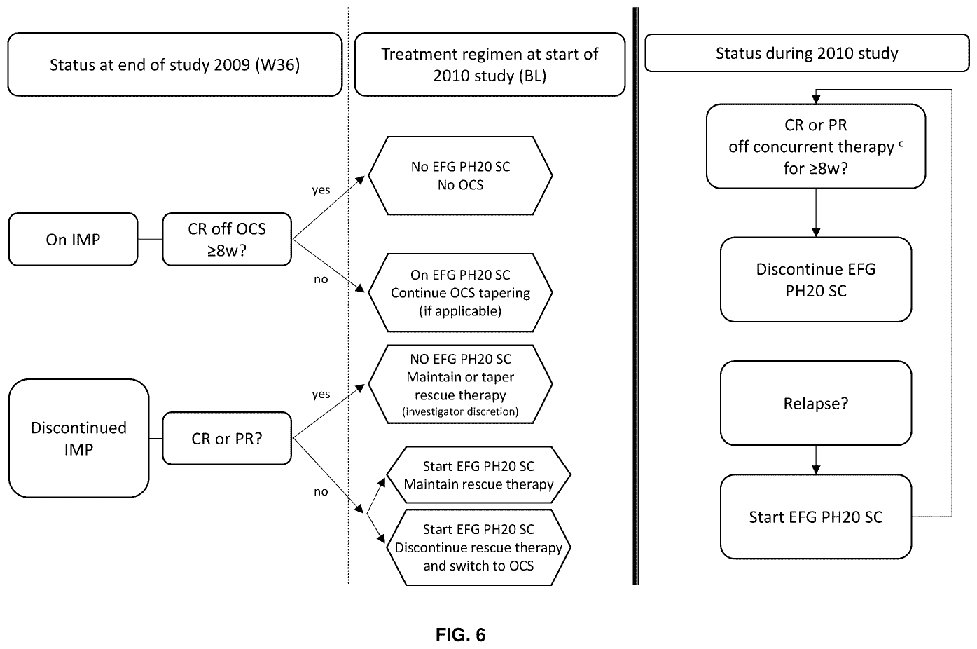

Efgartigimod PH20 SC will be administered in an open-label fashion according to the BP disease status of the participant. At the beginning of ARGX-113-2010, participants will be treated with efgartigimod PH20 SC according to their clinical status at the EoTP visit of ARGX-113-2009 as follows (illustrated in FIG. 6):
1. Participants who have been off OCS for >8 weeks at the ARGX-113-2009 EoTP visit will not receive efgartigimod PH20 SC treatment at the beginning of ARGX-113-2010. They will participate in ARGX-113-2010 observational assessments only (see Table S9) (without receiving efgartigimod PH20 SC or concurrent therapy) until they relapse or complete the study.
2. Participants who are still taking OCS at the ARGX-113-2009 EoTP visit or have been off OCS for <8 weeks at the ARGX-113-2009 EoTP visit will begin ARGX-113-2010 receiving efgartigimod PH20 SC therapy. The efgartigimod PH20 SC treatment regimen will vary depending on whether the participant received efgartigimod PH20 SC or placebo PH20 SC in ARGX-113-2009:
   a. Participants who received placebo PH20 SC in ARGX-113-2009—these participants will be administered two efgartigimod PH20 SC 1000 mg injections on both day 1 (baseline) and day 8, for total loading doses of efgartigimod PH20 SC 2000 mg on both day 1 and day 8. At once-weekly intervals thereafter (beginning on day 15), they will be administered single injections of efgartigimod PH20 SC 1000 mg.
   b. Participants who received efgartigimod PH20 SC in ARGX-113-2009—these participants will be administered one injection of efgartigimod PH20 SC 1000 mg and 1 injection of placebo PH20 SC on both day 1 (baseline) and day 8, for total doses of efgartigimod PH20 SC 1000 mg on both day 1 and day 8. At once-weekly intervals thereafter (beginning on day 15), they will be administered single injections of efgartigimod PH20 SC 1000 mg.
   Participants will be allocated to treatment regimen a or b using the interactive response technology (IRT) in order to maintain the blinding of treatment allocations from ARGX-113-2009. OCS tapering may proceed as described in Example 2 above. Efgartigimod PH20 SC therapy will subsequently be stopped once these participants have been in CR or PR while off OCS for ≥8 weeks.
3. Participants who have discontinued IMP, are receiving rescue therapy, and are in CR or PR at the ARGX-113-2009 EoTP visit will not receive efgartigimod PH20 SC treatment at the beginning of ARGX-113-2010. They will participate in ARGX-113-2010 observational assessments (see Table S9) until they relapse; their current rescue therapy regimen may be maintained or tapered at the discretion of the investigator.
4. Participants who have discontinued IMP, are receiving rescue therapy, but are not in CR or PR at the ARGX-113-2009 EoTP visit will begin ARGX-113-2010 on one of the following treatment regimens (upon advice from the investigator):
   a. Begin efgartigimod PH20 SC therapy (with loading doses of 2000 mg on day 1 and day 8 followed by maintenance doses of 1000 mg once weekly thereafter) while maintaining their current rescue therapy regimen. Once the participant's disease status begins to improve, rescue therapy may be tapered at the discretion of the investigator.
   b. Discontinue their current rescue therapy regimen, begin efgartigimod PH20 SC therapy (with loading doses of 2000 mg on day 1 and day 8 followed by maintenance doses of 1000 mg once weekly thereafter), and begin concurrent OCS therapy (once-daily dosage of prednisone [or equivalent] to be determined by the investigator based on clinical judgment). Once the participant's disease status begins to improve, OCS tapering will proceed as described Example 2 above.
   Treatment with efgartigimod PH20 SC 1000 mg once weekly will continue until the participant has been in CR or PR while off concurrent therapy for ≥8 weeks. Concurrent therapy is defined as any therapy administered for the treatment of BP other than efgartigimod PH20 SC. At that time, efgartigimod PH20 SC treatment will be stopped.

If a relapse occurs during study ARGX-113-2010, a new treatment course with efgartigimod PH20 SC may be initiated (at the discretion of the investigator)—beginning with loading doses of 2000 mg on day 1 and day 8 followed by maintenance doses of 1000 mg once weekly thereafter—until the participant has been in CR or PR while off concurrent therapy for ≥8 weeks.

During ARGX-113-2010, treatment with efgartigimod PH20 SC may be stopped when a participant has been in CR or PR while off concurrent therapy for >8 weeks. At that time, efgartigimod PH20 SC treatment will be stopped, and the participant will take part in ARGX-113-2010 observational assessments only (see Table S9) as long as CR or PR is sustained.

### Additional Considerations for Dosing Regimens and Assessment Schedules

Other BP treatment considerations include the following:
Efgartigimod PH20 SC monotherapy (without concurrent therapy) is allowed in this study at the discretion of the investigator.
In the event of a relapse, the efgartigimod PH20 SC treatment regimen (described above) may be initiated at the discretion of the investigator. If a relapse occurs while a participant is still taking concurrent therapy, efgartigimod PH20 SC may be added to the current treatment regimen. Thereafter, the concurrent therapy may be tapered according to clinical practice.
Transient lesions may be treated with TCS at the discretion of the investigator.

Participants who are off all systemic treatment will complete observation assessments only (see Table S9). Participants who are being treated with efgartigimod PH20 SC will be monitored more frequently than those who are not.

Efgartigimod PH20 SC injections that are scheduled between mandatory onsite visits (Table S9) may be administered by a home nurse; alternatively, they may be administered by the participant themselves or by their caregiver after they have completed efgartigimod PH20 SC (self-) administration refresher training and have been deemed competent to (self-) administer the IMP by site staff.

TABLE S9
ARGX-113-2010 schedule of activities

| | Study Period | | | | | | | | | | | | | |
|---|---|---|---|---|---|---|---|---|---|---|---|---|---|---|
| | | | | | | | | | | | | Follow-Up | | |
| | Treatment Period^{a} | | | | | | | | | | | Period^{b} | | UNS^{c} |
| | Study Week(s) | | | | | | | | | | | | | |
| | | | | | | | | | | | Wk 52 | | | |
| | | | Wks | | Wks | | Wks | | Wks | | EoTP | | | |
| | BL^{d} | Wk | 4 | Wk | 16 | Wk | 28 | Wk | 40 | Wk | (ETD; | | | |
| | (DX^{e}) | 2 | & 8 | 12 | & 20 | 24 | & 32 | 36 | & 44 | 48 | ESD)^{f} | F1 | F2 | UNS |
| | Visit Timing (Relative to DX) | | | | | | | | | | | | | |
| | | | +4 | | +16 | | +28 | | +40 | | EoTP | | | |
| | Wk | +2 | +8 | +12 | +20 | +24 | +32 | +36 | +44 | +48 | (ETD; | EoTP +4 | EoTP +8 | |
| | 0 | wks | wks | wks | wks | wks | wks | wks | wks | wks | ESD) | wks | wks | n/a |
| Informed consent^{g} | X | | | | | | | | | | | | | |
| Inclusion/exclusion criteria | X | | | | | | | | | | | | | |
| QoL questionnaires | X | | | X | | X | | X | | | X | | | |
| (EQ-5D-5L, DLQI, ABQoL) | | | | | | | | | | | | | | |
| Weight | X | X^{h} | X^{h} | X^{h} | X^{h} | X^{h} | X^{h} | X^{h} | X^{h} | X^{h} | X | | | X |
| Physical examination | X | X | X | X | X | X | X | X | X | X | X | X | X | X |
| Vital signs (blood pressure, | X | X | X | X | X | X | X | X | X | X | X | X | X | X |
| heart rate, body temperature) | | | | | | | | | | | | | | |
| Electrocardiogram (ECG) | X | | | | | X | | | | | X | | | |
| Urine pregnancy test | X | | X | X | X | X | X | X | X | X | X | | | |
| (local laboratory)ⁱ | | | | | | | | | | | | | | |
| Urinalysis | X | X | X | X | X | X | X | X | X | X | X | X | X | X |
| Clinical chemistry | X | X | X | X | X | X | X | X | X | X | X | X | X | X |
| and hematology | | | | | | | | | | | | | | |
| Pharmacokinetics | X | | X^{j} | X^{j} | X^{j} | X^{j} | X^{j} | X^{j} | X^{j} | X^{j} | X | X | X | X |
| Immunogenicity | X | | X^{j} | X^{j} | X^{j} | X^{j} | X^{j} | X^{j} | X^{j} | X^{j} | X | X | X | X |
| Pharmacodynamics (total IgG | X | | X | X | X | X | X | X | X | X | X | X | X | X |
| in serum; anti-BP180 & | | | | | | | | | | | | | | |
| anti-BP230 antibodies; IgE | | | | | | | | | | | | | | |
| in serum) | | | | | | | | | | | | | | |
| Substudy (selected sites): | Relative to the time of vaccination | | | | | | | | | | | | | |
| Vaccination antibody titers | | | | | | | | | | | | | | |
| (serum)^{k} | | | | | | | | | | | | | | |
| Substudy (selected sites): | Relative to the time of vaccination | | | | | | | | | | | | | |
| Vaccination antibody titers | | | | | | | | | | | | | | |
| (PBMCs)^{k} | | | | | | | | | | | | | | |
| IGA-BP | X | X | X | X | X | X | X | X | X | X | X | | | X |
| BPDAI | X | X | X | X | X | X | X | X | X | X | X | | | X |
| Itch NRS | X | X | X | X | X | X | X | X | X | X | X | | | X |
| BP disease status | X | X | X | X | X | X | X | X | X | X | X | X | X | X |
| Monitoring of concurrent | X | X | X | X | X | X | X | X | X | X | X | X | X | X |
| BP therapy^{l} | | | | | | | | | | | | | | |
| Glucocorticoid Toxicity Index | X | | | X | | X | | X | | | X | | | |
| (GTI; including C-GTI | | | | | | | | | | | | | | |
| and GTI-SL) | | | | | | | | | | | | | | |
| Concomitant | X | X | X | X | X | X | X | X | X | X | X | X | X | X |
| therapies/procedures | | | | | | | | | | | | | | |
| Adverse events | X | X | X | X | X | X | X | X | X | X | X | X | X | X |
| EFG PH20 SC administration^{m} | Once weekly (as required) | | | | | | | | | | | | | |
| EFG PH20 SC | At least once | | | | | | | | | | | | | |
| (self-)administration | | | | | | | | | | | | | | |
| refresher trainingⁿ | | | | | | | | | | | | | | |
| Rescue therapy evaluation/ | | | | | | | | | | | X | | | |
| initiation | | | | | | | | | | | (ETD)^{o} | | | |

ABQoL = Autoimmune Bullous Disease Quality of Life questionnaire;
BP = bullous pemphigoid;
BPDAI = Bullous Pemphigoid Disease Area Index;
BL = baseline;
C-GTI = composite Glucocorticoid Toxicity Index;
DLQI = Dermatology Life Quality Index;
EFG or EFG PH20 SC = efgartigimod coformulated with rHuPH20 for subcutaneous injection (dosing formulation);
EoS = end of study (week 52);
EoTP = end of treatment period (visit);
EQ-5D-5L = EuroQoL 5-dimension 5-level;
ESD = early study discontinuation;
ETD = early treatment discontinuation;
F1/F2 = follow-up visits 1 & 2;
GTI-SL = Glucocorticoid Toxicity Index Specific List;
IGA-BP = Investigator Global Assessment of Bullous Pemphigoid;
IgE = immunoglobulin type-E;
IgG = immunoglobulin type-G;
n/a = not applicable;
NRS = numerical rating scale;
OCS = oral corticosteroids;
PBMCs = peripheral blood mononuclear cells;
UNS = unscheduled visit
^{a}See “Treatment regimens” section above for descriptions of when the participant should be on efgartigimod PH20 SC and/or concurrent BP therapy. “Concurrent BP therapy” is defined as any therapy administered for the treatment of BP other than efgartigimod PH20 SC.
^{b}Participants are required to visit the study site at 4 weeks and 8 weeks after their EoTP visit (or ETD/ESW visits) for follow-up visit assessments.
^{c}An unscheduled visit should be performed if the participant has (or suspects that they have) new BP lesions, new AEs, or any other issues requiring site staff intervention. At these visits, the investigator and/or site staff will determine whether efgartigimod PH20 SC therapy must be started/resumed.
^{d}The baseline visit will occur on the same day as the end-of-treatment period (EoTP) visit of study ARGX-113-2009. All baseline assessments will be performed before administration of efgartigimod PH20 SC (if applicable).
^{e}Day X (DX) is defined as the day that the participant either begins or resumes treatment with efgartigimod PH20 SC during the study.
Note:
Day X can occur at baseline and can also occur on multiple occasions throughout the study.
^{f}Participants who are permanently discontinued from efgartigimod PH20 SC therapy before the EoTP visit (ETD) or are permanently discontinued from the study before the EoTP visit (ESW) must visit the study site to complete the week 52 (EoTP) assessments.
^{g}Participants must provide signed informed consent before any study-related activities are performed.
^{h}Only measured if the participant is being administered efgartigimod PH20 SC or OCS.
ⁱConducted in women of childbearing potential only
^{j}These pharmacokinetics and immunogenicity samples are only collected when the participant is being treated with efgartigimod PH20 SC.
^{k}Participants who took part in this substudy during ARGX-113-2009 can still have post-vaccination samples collected in ARGX-113-2010 (relative to the timing of the vaccination). Additionally, if the participant receives a vaccination during ARGX-113-2010, pre- and post-vaccination samples can be taken as described in the ARGX-113-2009 protocol.
^{l}“Concurrent BP therapy” is defined as any therapy administered for the treatment of BP other than efgartigimod PH20 SC.
^{m}Efgartigimod PH20 SC will be administered once weekly by subcutaneous injection. For doses that occur between scheduled onsite visits, the participant can choose to administer the injection themselves (if they have successfully completed efgartigimod PH20 SC [self-]administration training), have their caregiver administer the injection (if their caregiver has successfully completed efgartigimod PH20 SC [self-]administration training), have it administered by a home nurse, or visit the study site for the injection. During treatments that are administered onsite or by a home nurse, the participant should be monitored for safety for at least 1 hour after their first dose of efgartigimod PH20 SC, and then at least 15 minutes after all subsequent doses. Week 52 is the last timepoint that participants can receive doses of efgartigimod PH20 SC during this study.
ⁿParticipants and/or their caregivers will be invited to receive at least 1 refresher training course for efgartigimod PH20 SC (self-)administration. Training may continue or be repeated until the participant (or caregiver) is deemed competent to (self-)administer efgartigimod PH20 SC.
^{o}Participants who are permanently discontinued from efgartigimod PH20 SC therapy will be evaluated (by the investigator) to determine whether they should begin rescue therapy.

### B. Study Population

Prospective approval of protocol deviations to recruitment and enrollment criteria, also known as protocol waivers or exemptions, is not permitted.

### Inclusion Criteria

Participants are eligible to be included in the study only if all of the following criteria apply:
The participant completed the week 36 visit of ARGX-113-2009.
The participant is willing and able to do the following:
   a. understand the requirements of the study
   b. provide written informed consent (including consent for the use and disclosure of research-related health information)
   c. comply with the study protocol procedures (including required study visits).
Contraceptive use by men and women should be consistent with local regulations regarding the methods of contraception for those participating in clinical studies and:
   a. Male participants must agree to use an acceptable method of contraception from the time that the informed consent form is signed until the date of their last dose of IMP.
   b. Women of childbearing potential must have a negative serum pregnancy test at screening and a negative urine pregnancy test at baseline before study intervention can be administered; and must agree to use at least one contraception method from the time that the informed consent form is signed until the date of their last dose of IMP (a woman is considered to be of childbearing potential unless she is either postmenopausal or surgically sterilized).

### Exclusion Criteria

Participants are excluded from the study if any of the following criteria apply:
Have clinical evidence of other significant serious diseases, had a recent surgery, or have any other condition that, in the opinion of the investigator, could confound the results of the study or put the participant at undue risk.
Pregnant or lactating females and those who intend to become pregnant at any time from signing the ICF until they have received their last dose of the IMP.
Known hypersensitivity to any of the components of the administered treatments.

### Statistical Methodology

All safety endpoints will be reported using descriptive statistics in the form of summary tables. Wherever it is suitable, respective graphical displays will be presented along with the summary tables.

All efficacy endpoints will be evaluated descriptively and presented as summary tables. Summaries for continuous measures will include the number of observations (n), mean, SE, median, minimum, and maximum. For categorical variables, summaries will include sample size, frequencies, and percentages.

All summaries and plots will be produced both separately by the treatment arm in the core study (ARGX-113-2009) and pooling all subjects.

### C. Objectives and Endpoints

Study objectives and related endpoints are described in Table S10 below.

TABLE S10
Objectives and endpoints

| | |
|---|---|
| Objectives | Endpoints |

Primary

| | |
|---|---|
| To assess the safety of treatment with | Incidence and severity of TEAEs, AESIs, and SAEs |
| efgartigimod PH20 SC in participants with | by SOC and PT |
| bullous pemphigoid (BP) | Incidence and severity of changes in vital signs, |
| | physical examinations, ECGs, and clinical |
| | laboratory safety evaluations |

Secondary

| | |
|---|---|
| To assess the efficacy of treatment with | Proportion of participants who are in CR or PR |
| efgartigimod PH20 SC in participants with | and have stopped all BP therapy (both efgartigimod |
| BP | PH20 SC and concurrent BP therapy) |
| To assess the durability of response after | Duration that participants remain in CR or PR after |
| treatment with efgartigimod PH20 SC in | stopping all BP therapy (both efgartigimod |
| participants with BP | PH20 SC and concurrent BP therapy) |
| | Proportion of participants who relapse while |
| | on efgartigimod PH20 SC therapy |
| | Duration of time to relapse among participants who |
| | relapse while on efgartigimod PH20 SC therapy |
| | Proportion of participants who fail on treatment |
| | with efgartigimod PH20 SC therapy |
| | Proportion of participants who relapse but |
| | subsequently achieve CR or PR with efgartigimod |
| | PH20 SC therapy |
| | Duration that participants are in CR or PR while off |
| | concurrent BP therapy |
| | Proportion of participants who develop non- |
| | transient lesions while off all BP therapy (both |
| | efgartigimod PH20 SC and concurrent BP therapy) |
| | Proportion of participants who require rescue |
| | therapy (including treatments other than OCS |
| | or TCS) |
| | Time to discontinuation of efgartigimod PH20 SC |
| | BPDAI activity scores over time |
| | IGA-BP scores over time |
| | Itch NRS scores over time |
| To assess glucocorticoid-associated | Glucocorticoid Toxicity Index (GTI) scores, |
| morbidity-and evaluate the impact of | including scores for the GTI-AIS, GTI-CWS, |
| efgartigimod PH20 SC on reducing | and GTI-SL |
| glucocorticoid toxicity-in participants with | |
| BP | |
| To evaluate the effects of | EQ-5D-5L scores over time |
| efgartigimod PH20 SC on quality of life | DLQI scores over time |
| (QoL) in participants with BP | ABQoL scores over time |
| To evaluate the PK, PD, and immunogenicity | Efgartigimod serum concentrations |
| of efgartigimod PH20 SC in participants with | Total IgG serum levels |
| BP | Anti-BP180 and anti-BP230 antibodies |
| | Antidrug antibodies (ADA) to efgartigimod (serum) |
| | Antibodies produced against rHuPH20 (plasma) |

Exploratory

| | |
|---|---|
| To explore the feasibility of participants | Proportions of participants (or their caregivers) who |
| or their caregivers (self-)administering | successfully (self-)administer |
| efgartigimod PH20 SC | efgartigimod PH20 SC |
| | Number of training sessions required for the |
| | participant (and/or their caregiver) to be deemed |
| | qualified to (self-)administer efgartigimod PH20 SC |
| | Number of efgartigimod PH20 SC |
| | (self-)administrations performed at participants' |
| | homes |

ABQoL = Autoimmune Bullous Disease Quality of Life questionnaire;
AE = adverse event;
AESI = adverse event of special interest;
AIS = aggregate improvement score;
BP = bullous pemphigoid;
BPDAI = Bullous Pemphigoid Disease Area Index;
CWS = cumulative worsening score;
DLQI = Dermatology Life Quality Index;
ECG = electrocardiogram;
EFG = efgartigimod;
EFG PH20 SC = efgartigimod with rHuPH20 (coformulation) for SC injection;
EQ-5D-5L = EuroQoL 5-Dimension 5-Level;
GTI = Glucocorticoid Toxicity Index;
GTI-SL = Glucocorticoid Toxicity Index Specific List;
IGA-BP = Investigator Global Assessment of Bullous Pemphigoid;
IgE = immunoglobulin type-E;
IgG = immunoglobulin type-G;
Itch NRS = Itch Numerical Rating Scale;
OCS = oral corticosteroid (oral prednisone or an alternate corticosteroid of equivalent dose strength);
PBO PH20 SC = placebo with rHuPH20 (coformulation) for SC injection;
PD = pharmacodynamics;
PK = pharmacokinetics;
PR = partial remission;
PT = preferred term;
QoL = quality of life;
rHuPH20 = recombinant human hyaluronidase PH20;
SAE = serious adverse event;
SOC = system organ class;
TCS = topical corticosteroids;
TEAE = treatment-emergent adverse event

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. A method of treating bullous pemphigoid (BP) in a subject in need thereof, the method comprising administering to the subject an effective amount of a human neonatal Fc receptor (FcRn) antagonist.

2. The method of claim 1, wherein the FcRn antagonist comprises two, three, or four FcRn binding regions.

3. The method of claim 1 or 2, wherein the FcRn antagonist comprises or consists of a variant Fc region or FcRn binding fragment thereof.

4. The method of claim 3, wherein the variant Fc region or FcRn binding fragment thereof binds to FcRn with a higher affinity at pH 6.0 as compared to a corresponding wild-type Fc region.

5. The method of claim 3 or 4, wherein the variant Fc region or FcRn binding fragment thereof binds to FcRn with a higher affinity at pH 7.4 as compared to a corresponding wild-type Fc region.

6. The method of any one of claims 3-5, wherein the variant Fc region comprises or consists of a first Fc domain and a second Fc domain which form a homodimer or heterodimer.

7. The method of claim 6, wherein the first Fc domain and/or the second Fc domain comprise amino acids Y, T, E, K, and F at EU positions 252, 254, 256, 433, and 434, respectively.

8. The method of claim 6 or 7, wherein the first Fc domain and/or second Fc domain comprise amino acids Y, T, E, K, F, and Y at EU positions 252, 254, 256, 433, 434, and 436, respectively.

9. The method of any one of claims 6-8, wherein the first Fc domain and/or the second Fc domain comprise an amino acid sequence independently selected from the group consisting of SEQ ID NO: 1, SEQ ID NO: 2, SEQ ID NO: 3, and SEQ ID NO: 30.

10. The method of any one of claims 6-9, wherein the first Fc domain and the second Fc domain comprise an amino acid sequence independently selected from the group consisting of SEQ ID NO: 1, SEQ ID NO: 2, SEQ ID NO: 3, and SEQ ID NO: 30.

11. The method of any one of claims 1-10, wherein the FcRn antagonist is efgartigimod.

12. The method of claim 1, wherein the FcRn antagonist is an anti-FcRn antibody.

13. The method of any one of claims 1-12, wherein the FcRn antagonist is administered to the subject at a fixed dose of 200 mg to 20,000 mg or at a dose of 2 mg/kg to 200 mg/kg.

14. The method of any one of claims 1-13, wherein the FcRn antagonist is administered subcutaneously once weekly, once every two weeks, once every three weeks, once every four weeks, once monthly, or once every six weeks.

15. The method of claim 14, wherein the FcRn antagonist is administered subcutaneously at a fixed dose of 200 mg to 20,000 mg once weekly, once every two weeks, once every three weeks, once every four weeks, once monthly, or once every six weeks.

16. The method of claim 14 or 15, wherein the FcRn antagonist is administered subcutaneously at a fixed dose of 1000 mg or 2000 mg once weekly, once every two weeks, once every three weeks, once every four weeks, once monthly, or once every six weeks.

17. The method of claim 16, wherein the FcRn antagonist is first administered subcutaneously at a fixed dose of about 1000 mg twice on the same day.

18. The method of any one of claims 1-16, wherein the FcRn antagonist is administered subcutaneously at a fixed dose of about 2000 mg for the first two administrations and is subsequently administered subcutaneously at a fixed dose of about 1000 mg.

19. The method of claim 18, wherein the FcRn antagonist is administered subcutaneously at a fixed dose of 2000 mg for the first two administrations and is subsequently administered subcutaneously at a fixed dose of 1000 mg.

20. The method of any one of claims 1-19, wherein the first two administrations of the FcRn antagonist are subcutaneous administrations at a fixed dose of about 1000 mg administered twice on the same day.

21. The method of claim 20, wherein the first two administrations of the FcRn antagonist are subcutaneous administrations at a fixed dose of 1000 mg administered twice on the same day.

22. The method of claim 11, wherein the efgartigimod is administered subcutaneously at a fixed dose of 800 to 1200 mg once weekly.

23. The method of claim 11, wherein the efgartigimod is administered subcutaneously at a fixed dose of about 1000 mg once weekly.

24. The method of claim 11, wherein the efgartigimod is administered subcutaneously at a fixed dose of 1000 mg once weekly.

25. The method of any one of claims 1-24, wherein the FcRn antagonist is co-formulated with hyaluronidase and administered subcutaneously.

26. The method of claim 25, wherein the hyaluronidase is recombinant human hyaluronidase PH20 (rHuPH20).

27. The method of claim 26, wherein the rHuPH20 is administered at a dose of 2000 U/mL once weekly.

28. The method of claim 26, wherein the rHuPH20 is administered at about 11,000 U or about 22,000 U once weekly.

29. The method of any one of claims 1-13, wherein the FcRn antagonist is administered intravenously once weekly or once every two weeks.

30. The method of claim 29, wherein the FcRn antagonist is administered intravenously at a dose of from 2 mg/kg to 200 mg/kg once weekly or once every two weeks.

31. The method of claim 29 or 30, wherein the FcRn antagonist is administered intravenously at a dose of from 3 mg/kg to 60 mg/kg once weekly or once every two weeks.

32. The method of any one of claims 29-31, wherein the FcRn antagonist is administered intravenously at a dose of 5 mg/kg once weekly or once every two weeks.

33. The method of any one of claims 29-31, wherein the FcRn antagonist is administered intravenously at a dose of 10 mg/kg once weekly or once every two weeks.

34. The method of any one of claims 29-31, wherein the FcRn antagonist is administered intravenously at a dose of 25 mg/kg once weekly or once every two weeks.

35. The method of any one of claims 29-34, wherein the FcRn antagonist is first administered intravenously and is subsequently administered subcutaneously.

36. The method of claim 35, wherein the FcRn antagonist is first administered intravenously and is subsequently administered subcutaneously at fixed dose of 200 mg to 20,000 mg once weekly, once every two weeks, once every three weeks, once every four weeks, once monthly, or once every six weeks.

37. The method of claim 36, wherein the FcRn antagonist is first administered intravenously and is subsequently administered subcutaneously at fixed dose of 1000 mg or 2000 mg once weekly, once every two weeks, once every three weeks, once every four weeks, once monthly, or once every six weeks.

38. The method of any one of claims 1-37, wherein the FcRn antagonist is administered for 52 weeks or less.

39. The method of any one of claims 1-38, wherein the FcRn antagonist is administered for 36 weeks or less.

40. The method of any one of claims 1-39, wherein the FcRn antagonist is administered for at least 26 weeks.

41. The method of any one of claims 1-37, wherein the FcRn antagonist is administered for at least 36 weeks.

42. The method of any one of claims 1-41, further comprising administering to the subject a dose of corticosteroid.

43. The method of claim 42, wherein the dose of corticosteroid is reduced after disease control or complete remission has been achieved.

44. The method of claim 43, wherein the dose of corticosteroid is reduced after disease control has been sustained for at least two weeks.

45. The method of claim 44, wherein, following two weeks of sustained disease control, the dose of corticosteroid is decreased from a prednisone equivalent dose of 0.5 mg/kg/day to a prednisone equivalent dose of 0.3 mg/kg/day, from a prednisone equivalent dose of 0.3 mg/kg/day to a prednisone equivalent dose of 0.2 mg/kg/day, from a prednisone equivalent dose of 0.2 mg/kg/day to a prednisone equivalent dose of 0.15 mg/kg/day, and from a prednisone equivalent dose of 0.15 mg/kg/day to a prednisone equivalent dose of 0.1 mg/kg/day.

46. The method of claim 44, wherein, following two weeks of sustained disease control, the dose of corticosteroid is decreased from a prednisone equivalent dose of 0.75 mg/kg/day to a prednisone equivalent dose of 0.5 mg/kg/day, from a prednisone equivalent dose of 0.5 mg/kg/day to a prednisone equivalent dose of 0.3 mg/kg/day, from a prednisone equivalent dose of 0.3 mg/kg/day to a prednisone equivalent dose of 0.2 mg/kg/day, from a prednisone equivalent dose of 0.2 mg/kg/day to a prednisone equivalent dose of 0.15 mg/kg/day and from a prednisone equivalent dose of 0.15 mg/kg/day to a prednisone equivalent dose of 0.1 mg/kg/day.

47. The method of claim 44, wherein, following two weeks of sustained disease control, the dose of corticosteroid is decreased from a prednisone equivalent dose of 1 mg/kg/day to a prednisone equivalent dose of 0.75 mg/kg/day, from a prednisone equivalent dose of 0.75 mg/kg/day to a prednisone equivalent dose of 0.5 mg/kg/day, from a prednisone equivalent dose of 0.5 mg/kg/day to a prednisone equivalent dose of 0.3 mg/kg/day, from a prednisone equivalent dose of 0.3 mg/kg/day to a prednisone equivalent dose of 0.2 mg/kg/day, from a prednisone equivalent dose of 0.2 mg/kg/day to a prednisone equivalent dose of 0.15 mg/kg/day, and from a prednisone equivalent dose of 0.15 mg/kg/day to a prednisone equivalent dose of 0.1 mg/kg/day.

48. The method of any one of claim 45-47, wherein each dose of corticosteroid is maintained for at least two weeks.

49. The method of claim 48, wherein each dose of corticosteroid is maintained for at least two weeks provided that no new lesions appear.

50. The method of any one of claims 45-49, further comprising reducing the daily dose of corticosteroid by a prednisone equivalent dose of 2.5 mg every two weeks for one or more two-week periods.

51. The method of claim 50, wherein the daily dose of corticosteroid is decreased by a prednisone equivalent dose of 2.5 mg every two weeks for one or more two-week periods until the daily dose of corticosteroid is zero, thereby resulting in discontinuation of corticosteroid therapy.

52. The method of claim 50 or 51, wherein each daily dose is maintained for two weeks provided that no new lesions appear.

53. The method of any one of claims 45-49, further comprising reducing the daily dose of corticosteroid by a prednisone equivalent dose of 1 mg every week for one or more one-week periods.

54. The method of claim 53, wherein the daily dose of corticosteroid is decreased by a prednisone equivalent dose of 1 mg every week for one or more one-week periods until the daily dose of corticosteroids is zero, thereby resulting in discontinuation of corticosteroid therapy.

55. The method of claim 53 or 54, wherein each daily dose is maintained for one week provided that no new lesions appear.

56. The method of any one of claims 42-55, wherein the corticosteroid is prednisone.

57. The method of any one of claims 1-56, wherein the BP is selected from the group consisting of newly diagnosed BP, relapsing BP, and refractory BP.

58. The method of any one of claims 1-57, wherein the subject is diagnosed with moderate-to-severe BP, mild BP, moderate BP, or severe BP.

59. The method of any one of claims 1-58, wherein the subject has a detectable serum level of an anti-BP180 antibody or an anti-BP230 antibody or both.

60. The method of claim 59, wherein the subject shows a reduction in serum level of the anti-BP180 antibody or anti-BP230 antibody following administration of the FcRn antagonist.

61. The method of claim 60, wherein the reduction in the serum level of the anti-BP180 antibody or anti-BP230 antibody following administration of the FcRn antagonist is at least 10%, at least 25%, at least 50%, at least 75%, at least 80%, at least 90%, at least 95%, at least 96%, at least 97%, at least 98%, or at least 99% of the subject's serum level of the anti-BP180 antibody or anti-BP230 antibody prior to administration of the FcRn antagonist.

62. The method of claim 60 or 61, wherein the serum level of the anti-BP180 antibody or anti-BP230 antibody is undetectable after administration of the FcRn antagonist.

63. The method of any one of claims 1-62, wherein the FcRn antagonist is administered until disease control, partial remission, or complete remission is achieved.

64. The method of any one of claims 1-63, wherein the subject achieves complete remission.

65. The method of any one of claims 1-64, wherein complete remission is sustained in the subject for at least 2 months.

66. The method of claim 65, wherein complete remission is sustained in the subject for least 6 months.

67. The method of any one of claims 63-66, wherein the subject remains in complete remission in the absence of oral corticosteroids.

68. The method of claim 67, wherein the subject remains in complete remission in the absence of oral corticosteroids for at least eight weeks.

69. The method of claim 68, wherein the subject remains in complete remission in the absence of corticosteroids for at least eight weeks at the end of a 36-week treatment period.

70. The method of any one of claims 1-62, wherein BP relapse is prevented after achieving remission from BP, wherein BP relapse is prevented for at least 36 weeks after initiation of the FcRn antagonist administration.

71. The method of any one of claims 1-70, wherein the subject has an IGA-BP score of 0 or 1 after administration of the FcRn antagonist.

72. The method of claim 71, wherein the subject has an IGA-BP score of 0 or 1 in the absence of oral corticosteroids for at least eight weeks.

73. The method of claim 72, wherein the subject has an IGA-BP score of 0 or 1 in the absence of oral corticosteroids for at least eight weeks at the end of a 36-week treatment period.

74. The method of any one of claims 63-70, wherein the subject has an IGA-BP score of 0 after administration of the FcRn antagonist.

75. The method of claim 74, wherein the subject has an IGA-BP score of 0 in the absence of oral corticosteroids for at least eight weeks.

76. The method of claim 75, wherein the subject has an IGA-BP score of 0 in the absence of oral corticosteroids for at least eight weeks at the end of a 36-week treatment period.

77. An FcRn antagonist for use in the treatment of BP, wherein the treatment is performed according to the method of any one of the previous claims.

78. An FcRn antagonist for use in the manufacture of a medicament for the treatment of BP, wherein the treatment is performed according to the method of any one of the previous claims.

79. A method for monitoring efficacy of treatment of BP in a subject following treatment with a first FcRn antagonist, the method comprising:
a) measuring in vitro a serum level of an anti-BP180 antibody and/or anti-BP230 antibody in a blood sample taken from the subject; and
b) comparing the serum level of the anti-BP180 antibody and/or anti-BP230 antibody to a reference value associated with BP in the subject,
wherein the treatment is not effective if the serum level of the anti-BP180 antibody and/or anti-BP230 antibody in the sample is greater than or equal to the reference value, and wherein the treatment is effective if the serum level of the anti-BP180 antibody and/or anti-BP230 antibody is less than the reference value.

80. A method of treating BP in a subject that has received a first FcRn antagonist and is receiving a corticosteroid dosing regimen, the method comprising:
a) administering to the subject an effective amount of a second FcRn antagonist;
b) measuring in vitro a serum level of an anti-BP180 antibody and/or anti-BP230 antibody in a blood sample taken from the subject; and
c) comparing the serum level of the anti-BP180 antibody and/or anti-BP230 antibody to a reference value associated with BP in the subject,
wherein the corticosteroid dosing regimen is maintained if the serum level of the anti-BP180 antibody and/or anti-BP230 antibody in the sample is greater than or equal to the reference value, or wherein the corticosteroid dosing regimen is tapered if the serum level of the anti-BP180 antibody and/or anti-BP230 antibody is less than the reference value.

81. A method for treating BP in a subject comprising:
(a) administering to the subject one or more initial doses of an effective amount of a first FcRn antagonist; and
(b) administering to the subject one or more further doses of an effective amount of a second FcRn antagonist if the serum level of an anti-BP180 antibody and/or anti-BP230 antibody in the subject after step (a) is greater than or equal to a reference value associated with BP in the subject, or discontinuing treatment with the first FcRn antagonist if the serum level of the anti-BP180 antibody and/or anti-BP230 antibody in the subject after step (a) is less than a reference value associated with active disease in the subject.

82. A method for treating BP in a subject, the method comprising: administering to the subject an effective amount of a second FcRn antagonist,
wherein the BP has relapsed in the subject following prior therapy with a first FcRn antagonist, and wherein the subject has a serum level of an anti-BP180 antibody and/or anti-BP230 antibody that is greater than or equal to a reference value associated with BP in the subject.

83. A method for determining if a subject that has previously been treated for BP using a first FcRn antagonist requires further treatment with a second FcRn antagonist, the method comprising:
a) measuring in vitro the serum level of an anti-BP180 antibody and/or anti-BP230 antibody in a blood sample taken from the subject; and
b) comparing the serum level of the anti-BP180 antibody and/or anti-BP230 antibody to a reference value associated with BP in the subject,
wherein if the serum level of the anti-BP180 antibody and/or anti-BP230 antibody in the sample is greater than or equal to the reference value, then the subject is in need of further treatment with the second FcRn antagonist.

84. The method of any one of claims 79-83, wherein the subject was previously treated with the first FcRn antagonist at a fixed dose of about 200 mg to about 20,000 mg or at a dose of 2 mg/kg to 200 mg/kg.

85. The method of any one of claims 79-83, wherein the subject was previously treated with the first FcRn antagonist at a dose of from 20 mg/kg to 100 mg/kg, administered intravenously.

86. The method of claim 84 or 85, wherein the subject was previously treated with the first FcRn antagonist at a dose of from 3 mg/kg to 60 mg/kg, administered intravenously.

87. The method of any one of claims 80-83, wherein the effective amount of the second FcRn antagonist is a higher dose than the previous treatment with the first FcRn antagonist.

88. The method of any one of claims 80-83, wherein the effective amount of the second FcRn antagonist is a lower dose than the previous treatment with the first FcRn antagonist.

89. The method of any one of claims 80-83, wherein the effective amount of the second FcRn antagonist is administered at a fixed dose of about 200 mg to about 20,000 mg or at a dose of 2 mg/kg to 200 mg/kg.

90. The method of claim 89, wherein the effective amount of the second FcRn antagonist is administered subcutaneously at a fixed dose of about 750 mg to about 3000 mg once weekly, once every two weeks, once every three weeks, once every four weeks, once monthly, or once every six weeks.

91. The method of claim 90, wherein the effective amount of the second FcRn antagonist is administered subcutaneously at a fixed dose of about 1000 mg or about 2000 mg once weekly, once every two weeks, once every three weeks, once every four weeks, once monthly or once every six weeks.

92. The method of any one of claims 80-83, wherein the first FcRn antagonist and the second FcRn antagonist are each the same FcRn antagonist.

93. The method of any one of claims 80-83, wherein the first FcRn antagonist and the second FcRn antagonist are each a different FcRn antagonist.

94. The method of claim 93, wherein the FcRn antagonist comprises or consists of a variant Fc region or FcRn binding fragment thereof.

95. The method of claim 94, wherein the first FcRn antagonist or the second FcRn antagonist comprises or consists of a variant Fc region or FcRn binding fragment thereof.

96. The method of claim 94 or 95, wherein the variant Fc region or FcRn binding fragment thereof binds to FcRn with a higher affinity at pH 6.0 and/or at pH 7.4 as compared to a corresponding wild-type Fc region.

97. The method of claim 94 or 95, wherein the variant Fc region comprises or consists of two Fc domains which form a homodimer or heterodimer.

98. The method of any one of claim 92, 94, 96, or 97, wherein the FcRn antagonist is an Fc region comprising amino acids Y, T, E, K, F, and Y at EU positions 252, 254, 256, 433, 434, and 436, respectively.

99. The method of any one of claim 93, 95, 96, or 97, wherein the first FcRn antagonist or the second FcRn antagonist is an Fc region comprising amino acids Y, T, E, K, F, and Y at EU positions 252, 254, 256, 433, 434, and 436, respectively.

100. The method of claim 98, wherein the FcRn antagonist is efgartigimod.

101. The method of claim 99, wherein the first FcRn antagonist or the second FcRn antagonist is efgartigimod.

102. The method of any one of claim 92, 94, 96, or 97, wherein the amino acid sequence of at least one of the Fc domains is selected from the group consisting of SEQ ID NO: 1, SEQ ID NO: 2, and SEQ ID NO: 3.

103. The method of any one of claim 93, 95, 96, or 97, wherein the amino acid sequence of both of the Fc domains is independently selected from the group consisting of SEQ ID NO: 1, SEQ ID NO: 2, and SEQ ID NO: 3.

104. The method of claim 102, wherein the first FcRn antagonist is an anti-FcRn antibody and the second FcRn antagonist is efgartigimod.

105. The method of claim 102, wherein the first FcRn antagonist is an anti-FcRn antibody and the second FcRn antagonist comprises the amino acid sequence of SEQ ID NO: 1, 2, or 3.

106. The method of claim 104 or 105, wherein the patient has not been previously treated with efgartigimod.

107. The method of any one of claims 79-106, wherein the subject has one or more physical symptoms of BP following treatment with the first FcRn antagonist.

108. The method of any one of claims 79-107, wherein the subject has a serum level of an anti-BP180 antibody or anti-BP230 antibody that is associated with relapse of BP.

109. A method of treating BP in a subject in need thereof, the method comprising administering to the subject an effective amount of a human FcRn antagonist and a corticosteroid, wherein a cumulative oral dose of the corticosteroid administered to the subject does not exceed 100 mg/kg, does not exceed 75 mg/kg, does not exceed 50 mg/kg, or does not exceed 25 mg/kg.

110. A method of treating BP in a subject in need thereof, the method comprising administering to the subject an effective amount of a human FcRn antagonist and a corticosteroid, wherein an oral prednisone equivalent dose of the corticosteroid administered to the subject does not exceed 1 mg/kg/day, does not exceed 0.75 mg/kg/day, does not exceed 0.5 mg/kg/day, does not exceed 0.3 mg/kg/day, does not exceed 0.25 mg/kg/day, does not exceed 0.2 mg/kg/day, does not exceed 0.15 mg/kg/day, or does not exceed 0.1 mg/kg/day.

111. A method of treating BP in a subject in need thereof, the method comprising administering to the subject an effective amount of a human FcRn antagonist and a corticosteroid, wherein an oral prednisone equivalent dose of the corticosteroid administered to the subject does not exceed 12.5 mg/day or does not exceed 10 mg/day.


SEQUENCE LISTING (31 sequences)

SEQ ID NO: 1 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 216 aa, synthetic construct
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 5 - protein, 445 aa, synthetic construct
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 219 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 214 aa, synthetic construct
DIQMTQSPSSLSASVGDRVTITCKASDHINNWLAWYQQKPGQAPRLLISGATSLETGVPSRFSGSGTGKDYTLTISSLQPEDFATYYCQQYWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 444 aa, synthetic construct
QVQLVQSGAELKKPGASVKLSCKASGYTFTSYGISWVKQATGQGLEWIGEIYPRSGNTYYNEKFKGRATLTADKSTSTAYMELRSLRSEDSAVYFCARSTTVRPPGIWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 15 aa, synthetic construct
EPKSCDKTHTCPPCP

SEQ ID NO: 13 - protein, 227 aa, synthetic construct
  VARIANT 1: note = D or absent
  VARIANT 2: note = K or absent
  VARIANT 3: note = T or absent
  VARIANT 4: note = H or absent
  VARIANT 5: note = T or absent
  VARIANT 6: note = C or absent
  VARIANT 226: note = G or absent
  VARIANT 227: note = K or absent
XXXXXXPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPXX

SEQ ID NO: 14 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 15 - protein, 226 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 16 - protein, 225 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 17 - protein, 224 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 18 - protein, 225 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 19 - protein, 224 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 20 - protein, 223 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 21 - protein, 223 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 22 - protein, 222 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 23 - protein, 221 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 24 - protein, 221 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 25 - protein, 220 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 26 - protein, 219 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 27 - protein, 224 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 28 - protein, 223 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 29 - protein, 222 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 30 - protein, 222 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 31 - protein, 220 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP